(19) United States
(12) Patent Application Publication
(10) Pub. No.: US 2024/0156985 A1
(43) Pub. Date: May 16, 2024

(54) MUTATION-INDEPENDENT GENE KNOCK-IN THERAPY TARGETING 5' UTR

(71) Applicant: City University of Hong Kong, Kowloon (HK)

(72) Inventors: Wenjun Xiong, Kowloon (HK); Anh Duc Hoang, Hanoi (VN); Baoshan Liao, Kowloon (HK)

(21) Appl. No.: 17/985,765

(22) Filed: Nov. 11, 2022

(51) Int. Cl. A61K 48/00; A61K 31/7105; A61K 38/17; A61K 38/46; A61P 27/02; C12N 9/22; C12N 15/11; C12N 15/86; C12N 15/90

(52) U.S. Cl. CPC A61K 48/005; A61K 31/7105; A61K 38/177; A61K 38/465; A61P 27/02; C12N 9/22; C12N 15/11; C12N 15/86; C12N 15/907; C12N 2310/20; C12N 2750/14143; C12N 2750/14152; C12N 2750/14171; C12N 2800/80

(57) ABSTRACT

Novel 5′ untranslated region (UTR)-targeting gene knock-in (KI) compositions and methods of use are disclosed. The gene KI compositions and methods exploit homology-independent targeted integration (HITI)-mediated insertion of a wild-type coding sequence (CDS) into the 5′ UTR upstream of a translation initiation element of a mutated variant of the wild-type gene. The 5′ UTR-targeting gene KI therapy compositions and methods provide safer and more efficient gene insertion compared to other gene therapy approaches.

### SEQUENCE LISTING

The instant application contains a Sequence Listing which has been submitted in XML format and is hereby incorporated by reference in its entirety. Said XML copy, created on Nov. 7, 2022, is named “CTYU.P0034US Sequence Listing” and is 73,104 bytes in size.

### TECHNICAL FIELD

Aspects of the disclosure include at least the fields of cell biology, molecular biology, and medicine, including gene therapy.

### BACKGROUND

Despite the recent success of gene supplementation therapy for monogenic recessive diseases, therapeutic approaches to treat autosomal dominant disorders fall behind. Mutation-specific knockdown and/or knockout of the disease alleles is largely limited by off-target effects of RNA interference and the availability of protospacer adjacent motif (PAM) sites. Base editing or prime editing enables precise repair of the disease allele but is not versatile for diseases with high mutation heterogeneity.

For example, retinitis pigmentosa (RP) is an autosomal dominant photoreceptor degeneration disease with high genetic heterogeneity (>90 disease-causing genes including RHO (20-30% adRP), RP1 (5-10% adRP), RPRH2 (5% adRP), and IMPDH1 (>2% adRP)). The rhodopsin protein, encoded by the RHO gene, is an example of an RP-causing disease gene. Rhodopsin is the light-sensing G protein-coupled receptor that activates phototransduction in rod photoreceptors in the retina. RHO mutations are responsible for 20-30% of all adRP, and more than two hundred loss-of-function and gain-of-function RHO gene mutations have been identified, with the Rho^{P23H} (p.Pro23His, c.68C>A) mutation being the most common mutation in adRP patients. 1-3 While gene supplementation therapy has emerged as a promising treatment for autosomal recessive RP (arRP), it remains a challenge to treat autosomal dominant RP (adRP) due to the inefficient disruption of mutant alleles and the broad spectrum of loss-of-function and gain-of-function mutations to be addressed.

There exists a need for enhanced methods and compositions for treatment of autosomal dominant disorders.

### SUMMARY

The present invention is based on the discovery that novel 5′ untranslated region (UTR)-targeting gene knock-in (KI) compositions and methods can exploit homology-independent targeted integration (HITI)-mediated insertion of a wild-type coding sequence (CDS) into the 5′ untranslated region (UTR) upstream of a translation initiation element of a mutated variant of the wild-type gene. The 5′ UTR-targeting gene KI therapy compositions and methods provide surprising safety and efficiency insertion compared to other gene therapy approaches.

Provided herein, in some aspects, are methods for editing the genome of a cell, the method comprising contacting the cell with a composition comprising a nuclease and an exogenous nucleic acid encoding a knock-in cassette comprising a coding sequence for a wild-type gene and translation initiation and termination elements for expression of the wild-type gene, wherein the nuclease causes a break within a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid.

Also provided herein, in some aspects, are compositions comprising: a nuclease; and an exogenous nucleic acid encoding a knock-in cassette comprising a coding sequence for a wild-type gene and translation initiation and termination elements for expression of the wild-type gene; wherein, when introduced into a cell, the nuclease causes a break within a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid.

Also provided herein, in some aspects, are engineered cells comprising a genomic modification, wherein the genomic modification comprises integration of an exogenous nucleic acid encoding a knock-in cassette into the genome of the cell, wherein the knock-in cassette comprises a coding sequence for a wild-type gene, and wherein the exogenous nucleic acid encoding the knock-in cassette is integrated into a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid.

Also provided herein, in some aspects, are methods for treating or preventing an autosomal disorder in a subject identified as expressing a mutated gene variant, the method comprising introducing into a cell of the subject an effective amount of a composition comprising: a nuclease; and an exogenous nucleic acid encoding a knock-in cassette comprising a coding sequence for a wild-type gene and translation initiation and termination elements for expression of the wild-type gene; wherein the nuclease causes a break within a 5′ UTR of an endogenous nucleic acid encoding the mutated gene variant, wherein the endogenous nucleic acid encodes, in the 5′ to 3′ direction, the 5′ UTR, a translation initiation element for expression of the mutated gene variant, and a coding sequence for the mutated gene variant, wherein the nucleic acid encoding the knock-in cassette is integrated by homology-independent targeted integration into the 5′ UTR of the endogenous nucleic acid upstream (5′) of the translation initiation element encoded by the endogenous nucleic acid, and wherein integration of the nucleic acid encoding the knock-in cassette results in expression of the wild-type gene, and wherein expression of the wild-type gene results in decreased expression of the mutated gene variant.

Also provided herein, in some aspects, are compositions comprising: a Cas9 nuclease; an exogenous nucleic acid encoding a knock-in cassette comprising a coding sequence for a wild-type gene and translation initiation and termination elements for expression of the wild-type gene; and a guide molecule for the CRISPR/Cas nuclease to direct the Cas9 to a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and wherein, when introduced into a cell, the nuclease causes a break within the 5′ UTR of the endogenous nucleic acid encoding the mutated variant of the wild-type gene, and wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid.

In some aspects of the compositions and methods disclosed herein, the exogenous nucleic acid encoding the knock-in cassette is not integrated in-frame with the endogenous nucleic acid encoding the mutated variant of the wild-type gene. In some aspects of the compositions and methods disclosed herein, the exogenous nucleic acid encoding the knock-in cassette is integrated in-frame with the endogenous nucleic acid encoding the mutated variant of the wild-type gene. In some aspects, the knock-in cassette is not flanked by homology arms. In some aspects, integration of the exogenous nucleic acid encoding the knock-in cassette results in expression of the wild-type gene by the cell. In some aspects, expression of the wild-type gene by the cell inhibits expression of the mutated variant of the wild-type gene.

In some aspects of the compositions and methods disclosed herein, the nuclease is a CRISPR/Cas nuclease, and the method further comprises contacting the cell with a guide molecule for the CRISPR/Cas nuclease. In some aspects, the nuclease is a zinc finger nuclease (ZFN), a transcription activator-like effector nuclease (TALEN), or a meganuclease.

In some aspects of the compositions and methods disclosed herein, the nuclease is encoded by the same exogenous nucleic acid encoding the knock-in cassette, and wherein the exogenous nucleic acid is comprised in a vector. In some aspects, the nuclease is a CRISPR/Cas nuclease, and wherein the vector comprising the exogenous nucleic acid encoding the nuclease and the knock-in cassette further comprises a nucleic acid encoding a guide molecule for the CRISPR/Cas nuclease. In some aspects, the vector is a plasmid, a transposon, a cosmid, an artificial chromosome, a lipid nanoparticle, or a viral vector. In some aspects, the vector is a viral vector, and wherein the viral vector is an adeno-associated virus (AAV) vector, an adenovirus vector, a lentivirus vector, or a retrovirus vector. In specific aspects, the viral vector is an AAV vector.

In some aspects of the compositions and methods disclosed herein, the nuclease is encoded by a nucleic acid that is different from the exogenous nucleic acid encoding the knock-in cassette, and wherein the nucleic acid encoding the nuclease and the exogenous nucleic acid encoding the knock-in cassette are comprised in two different vectors. In some aspects, the nuclease is a CRISPR/Cas nuclease, and wherein the vector comprising the exogenous nucleic acid encoding the knock-in cassette further comprises a nucleic acid encoding a guide molecule for the CRISPR/Cas nuclease. In some aspects, the vectors are a plasmid, a transposon, a cosmid, an artificial chromosome, a lipid nanoparticle, or a viral vector. In some aspects, the vectors are a viral vector, and wherein the viral vector is an adeno-associated virus (AAV) vector, an adenovirus vector, a lentivirus vector, or a retrovirus vector. In specific aspects, the viral vector is an AAV vector.

In some aspects of the compositions and methods disclosed herein, the coding sequence for the wild-type gene is operably linked to a promoter. In some aspects, the promoter is an inducible promoter, a constitutive promoter, or a tissue-specific promoter.

In some aspects of the compositions and methods disclosed herein, the mutated variant of the wild-type gene (i.e., the mutated gene variant) is a dominant variant. In some aspects, the wild-type gene is the RHO gene. In some aspects of the compositions and methods disclosed herein, the mutated variant of the wild-type gene (i.e., the mutated gene variant) is a recessive variant.

Also disclosed herein, in some aspects, are methods comprising introducing into a subject a therapeutically effective amount of a composition disclosed herein; methods of expressing a wild-type gene (e.g., a wild-type gene from which a mutated gene variant is derived) in cells comprising introducing into a subject a therapeutically effective amount of a composition disclosed herein; and methods of decreasing the expression of a mutated variant of a wild-type gene (e.g., a wild-type gene from which a mutated gene variant is derived) in cells comprising introducing into a subject a therapeutically effective amount of a composition disclosed herein. In some aspects, the subject is a human. In some aspects, the subject is an animal. In some aspects, the subject was previously identified as having cells expressing a mutated variant of a wild-type gene. In some aspects, introduction of a therapeutically effective amount of the composition inhibits expression of the mutated variant of the wild-type gene. In some aspects, the mutated variant of the wild-type gene is a dominant variant. In specific aspects, the wild-type gene is the RHO gene, and the composition is introduced to the retina of the subject. In some aspects, the mutated variant of the wild-type gene is a recessive gene variant.

Also disclosed in specific aspects are methods of increasing expression of wild-type RHO gene in the retina of a subject, the method comprising introducing into the retina of the subject a therapeutically effective amount a composition disclosed herein. In some aspects, the subject was previously identified as having retinal cells expressing a mutated variant of the RHO gene. In some aspects, expression of the wild-type RHO gene inhibits expression of the mutated variant of the wild-type RHO gene. In some aspects, the subject is a human. In some aspects, the subject is an animal.

Also disclosed are the following Aspects 1-142 of the present disclosure.

Aspect 1 is a method for editing the genome of a cell, the method comprising contacting the cell with a composition comprising a nuclease and an exogenous nucleic acid encoding a knock-in cassette comprising a coding sequence for a wild-type gene and translation initiation and termination elements for expression of the wild-type gene, wherein the nuclease causes a break within a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid. Aspect 2 is the method of Aspect 1, wherein the exogenous nucleic acid encoding the knock-in cassette is not integrated in-frame with the endogenous nucleic acid encoding the mutated variant of the wild-type gene. Aspect 3 is the method of Aspect 1, wherein the exogenous nucleic acid encoding the knock-in cassette is integrated in-frame with the endogenous nucleic acid encoding the mutated variant of the wild-type gene. Aspect 4 is the method of any one of Aspects 1-3, wherein the knock-in cassette is not flanked by homology arms. Aspect 5 is the method of any one of Aspects 1-4, wherein integration of the exogenous nucleic acid encoding the knock-in cassette results in expression of the wild-type gene by the cell. Aspect 6 is the method of Aspect 5, wherein expression of the wild-type gene by the cell inhibits expression of the mutated variant of the wild-type gene. Aspect 7 is the method of any one of Aspects 1-6, wherein the nuclease is a CRISPR/Cas nuclease, and wherein the method further comprises contacting the cell with a guide molecule for the CRISPR/Cas nuclease. Aspect 8 is the method of any one of Aspects 1-6, wherein the nuclease is a zinc finger nuclease (ZFN), a transcription activator-like effector nuclease (TALEN), or a meganuclease. Aspect 9 is the method of any one of Aspects 1-8, wherein the nuclease is encoded by the same exogenous nucleic acid encoding the knock-in cassette, and wherein the exogenous nucleic acid is comprised in a vector. Aspect 10 is the method of Aspect 9, wherein the nuclease is a CRISPR/Cas nuclease, and wherein the vector comprising the exogenous nucleic acid encoding the nuclease and the knock-in cassette further comprises a nucleic acid encoding a guide molecule for the CRISPR/Cas nuclease. Aspect 11 is the method any Aspect 9 or Aspect 10, wherein the vector is a plasmid, a transposon, a cosmid, an artificial chromosome, a lipid nanoparticle, or a viral vector. Aspect 12 is the method of any one of Aspects 9-11, wherein the vector is a viral vector, and wherein the viral vector is an adeno-associated virus (AAV) vector, an adenovirus vector, a lentivirus vector, or a retrovirus vector. Aspect 13 is the method of Aspect 12, wherein the viral vector is an AAV vector. Aspect 14 is the method of any one of Aspects 1-8, wherein the nuclease is encoded by a nucleic acid that is different from the exogenous nucleic acid encoding the knock-in cassette, and wherein the nucleic acid encoding the nuclease and the exogenous nucleic acid encoding the knock-in cassette are comprised in two different vectors. Aspect 15 is the method of Aspect 14, wherein the nuclease is a CRISPR/Cas nuclease, and wherein the vector comprising the exogenous nucleic acid encoding the knock-in cassette further comprises a nucleic acid encoding a guide molecule for the CRISPR/Cas nuclease. Aspect 16 is the method of Aspect 14 or Aspect 15, wherein the vectors are a plasmid, a transposon, a cosmid, an artificial chromosome, a lipid nanoparticle, or a viral vector. Aspect 17 is the method of any one of Aspects 14-16, wherein the vectors are a viral vector, and wherein the viral vector is an adeno-associated virus (AAV) vector, an adenovirus vector, a lentivirus vector, or a retrovirus vector. Aspect 18 is the method of Aspect 17, wherein the viral vector is an AAV vector. Aspect 19 is the method of any one of Aspects 1-18, wherein the coding sequence for the wild-type gene is operably linked to a promoter. Aspect 20 is the method of Aspect 19, wherein the promoter is an inducible promoter, a constitutive promoter, or a tissue-specific promoter. Aspect 21 is the method of any one of Aspects 1-20, wherein the mutated variant of the wild-type gene is a dominant variant. Aspect 22 is the method of any one of Aspects 1-21, wherein the wild-type gene is the RHO gene. Aspect 23 is the method of any one of Aspects 1-20, wherein the mutated variant of the wild-type gene is a recessive variant.

Aspect 24 is an engineered cell comprising a genomic modification, wherein the genomic modification comprises integration of an exogenous nucleic acid encoding a knock-in cassette into the genome of the cell, wherein the knock-in cassette comprises a coding sequence for a wild-type gene, and wherein the exogenous nucleic acid encoding the knock-in cassette is integrated into a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid. Aspect 25 is the engineered cell of Aspect 24, wherein the knock-in cassette further comprises translation initiation and termination elements for expression of the wild-type gene. Aspect 26 is the engineered cell of Aspect 25, wherein the exogenous nucleic acid encoding the knock-in cassette is not integrated in-frame with the endogenous nucleic acid encoding the mutated variant of the wild-type gene. Aspect 27 is the engineered cell of Aspect 25, wherein the exogenous nucleic acid encoding the knock-in cassette is integrated in-frame with the endogenous nucleic acid encoding the mutated variant of the wild-type gene. Aspect 28 is the engineered cell of any one of Aspects 24-27, wherein the knock-in cassette is not flanked by homology arms. Aspect 29 is the engineered cell of any one of 24-28, wherein integration of the exogenous nucleic acid encoding the knock-in cassette results in expression of the wild-type gene. Aspect 30 is the engineered cell of Aspect 29, wherein expression of the wild-type gene inhibits expression of the mutated variant of the wild-type gene. Aspect 31 is the engineered cell of any one of Aspects 24-30, wherein the coding sequence for the wild-type gene is operably linked to a promoter. Aspect 32 is the engineered cell of Aspect 31, wherein the promoter is an inducible promoter, a constitutive promoter, or a tissue-specific promoter. Aspect 33 is the engineered cell of any one of Aspects 24-32, wherein the mutated variant of the wild-type gene is a dominant variant. Aspect 34 is the engineered cell of any one of Aspects 24-33, wherein the wild-type gene is the RHO gene. Aspect 35 is the engineered cell of any one of Aspects 24-32, wherein the mutated variant of the wild-type gene is a recessive variant.

Aspect 36 is a composition comprising: a nuclease; and an exogenous nucleic acid encoding a knock-in cassette comprising a coding sequence for a wild-type gene and translation initiation and termination elements for expression of the wild-type gene; wherein, when introduced into a cell, the nuclease causes a break within a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid. Aspect 37 is the composition of Aspect 36, wherein the knock-in cassette is not flanked by homology arms. Aspect 38 is the composition of Aspect 36 or Aspect 37, wherein the nuclease is a CRISPR/Cas nuclease, and wherein the method further comprises contacting the cell with a guide molecule for the CRISPR/Cas nuclease. Aspect 39 is the composition of Aspect 36 or Aspect 37, wherein the nuclease is a zinc finger nuclease (ZFN), a transcription activator-like effector nuclease (TALEN), or a meganuclease. Aspect 40 is the composition of any one of Aspects 36-39, wherein the nuclease is encoded by the same exogenous nucleic acid encoding the knock-in cassette, and wherein the exogenous nucleic acid is comprised in a vector. Aspect 41 is the composition of Aspect 40, wherein the nuclease is a CRISPR/Cas nuclease, and wherein the vector comprising the exogenous nucleic acid encoding the nuclease and the knock-in cassette further comprises a nucleic acid encoding a guide molecule for the CRISPR/Cas nuclease. Aspect 42 is the composition of Aspect 40 or Aspect 41, wherein the vector is a plasmid, a transposon, a cosmid, an artificial chromosome, a lipid nanoparticle, or a viral vector. Aspect 43 is the composition of any one of Aspects 40-42, wherein the vector is a viral vector, and wherein the viral vector is an adeno-associated virus (AAV) vector, an adenovirus vector, a lentivirus vector, or a retrovirus vector. Aspect 44 is the composition of Aspect 43, wherein the viral vector is an AAV vector. Aspect 45 is the composition of any one of Aspects 36-39, wherein the nuclease is encoded by a nucleic acid that is different from the exogenous nucleic acid encoding the knock-in cassette, and wherein the nucleic acid encoding the nuclease and the exogenous nucleic acid encoding the knock-in cassette are comprised in two different vectors. Aspect 46 is the composition of Aspect 45, wherein the nuclease is a CRISPR/Cas nuclease, and wherein the vector comprising the exogenous nucleic acid encoding the knock-in cassette further comprises a nucleic acid encoding a guide molecule for the CRISPR/Cas nuclease. Aspect 47 is the composition of Aspect 45 or Aspect 46, wherein the vector is a plasmid, a transposon, a cosmid, an artificial chromosome, a lipid nanoparticle, or a viral vector. Aspect 48 is the composition of any one of Aspects 45-47, wherein the vector is a viral vector, and wherein the viral vector is an adeno-associated virus (AAV) vector, an adenovirus vector, a lentivirus vector, or a retrovirus vector. Aspect 49 is the composition of Aspect 48, wherein the viral vector is an AAV vector. Aspect 50 is the composition of any one of Aspects 36-49, wherein the coding sequence for the wild-type gene is operably linked to a promoter. Aspect 51 is the composition of Aspect 50, wherein the promoter is an inducible promoter, a constitutive promoter, or a tissue-specific promoter. Aspect 52 is the composition of any one of Aspects 36-51, wherein the mutated variant of the wild-type gene is a dominant variant. Aspect 53 is the composition of any one of Aspects 38-52, wherein the wild-type gene is the RHO gene. Aspect 54 is the composition of any one of Aspects 36-51, wherein the mutated variant of the wild-type gene is a recessive variant. Aspect 55 is the composition of any one of Aspects 1-54, further comprising a pharmaceutically acceptable excipient.

Aspect 56 is a method comprising introducing into a subject a therapeutically effective amount of the composition of any one of Aspects 36-55. Aspect 57 is the method of Aspect 56, wherein the subject is a human or an animal. Aspect 58 is the method of Aspect 56 or Aspect 57, wherein the subject was previously identified as having cells expressing a mutated variant of a wild-type gene. Aspect 59 is the method of Aspect 58, wherein introduction of a therapeutically effective amount of the composition of any one of Aspects 36-55 inhibits expression of the mutated variant of the wild-type gene. Aspect 60 is the method of Aspect 58 or Aspect 59, wherein the mutated variant of the wild-type gene is a dominant variant. Aspect 61 is the method of any one of Aspects 58-60, wherein the wild-type gene is the RHO gene. Aspect 62 is the method of Aspect 61, wherein the composition is introduced to the retina of the subject. Aspect 63 is a method of expressing a wild-type gene in cells, the method comprising introducing the composition of any one of Aspects 36-55 into the cells. Aspect 64 is the method of Aspect 63, wherein the cells are human or animal cells. Aspect 65 is the method of Aspect 63 or Aspect 64, wherein the cells were previously determined to express a mutated variant of the wild-type gene. Aspect 66 is the method of Aspect 65, wherein expression of the wild-type gene inhibits expression of the mutated variant of the wild-type gene. Aspect 67 is the method of any one of Aspects 63-66, wherein the mutated variant of the wild-type gene is a dominant variant. Aspect 68 is the method of any one of Aspects 63-67, wherein the wild-type gene is the RHO gene. Aspect 69 is the method of Aspect 68, wherein the composition is introduced to the retinal cells of the subject. Aspect 70 is the method of any one of Aspects 63-66, wherein the mutated variant of the wild-type gene is a recessive variant.

Aspect 71 is a method of decreasing the expression of a mutated variant of a wild-type gene in cells, the method comprising introducing the composition of any one of Aspects 36-55 into the cells. Aspect 72 is the method of Aspect 71, wherein the cells are human or animal cells. Aspect 73 is the method of Aspect 71 or Aspect 72, wherein the mutated variant of the wild-type gene is a dominant variant. Aspect 74 is the method of any one of Aspects 71-73, wherein the wild-type gene is the RHO gene. Aspect 75 is the method of Aspect 74, wherein the composition is introduced to the retinal cells of the subject. Aspect 76 is the method of any one of Aspects 71-73, wherein the mutated variant of the wild-type gene is a recessive variant.

Aspect 77 is a method of increasing expression of wild-type RHO gene in the retina of a subject, the method comprising introducing into the retina of the subject a therapeutically effective amount of the composition of any one of Aspects 1-55. Aspect 78 is the method of Aspect 77, wherein the subject was previously identified as having retinal cells expressing a mutated variant of the RHO gene. Aspect 79 is the method of Aspect 78, wherein expression of the wild-type RHO gene inhibits expression of the mutated variant of the wild-type RHO gene. Aspect 80 is the method of any one of Aspects 77-80, wherein the subject is a human or an animal.

Aspect 81 is a method for treating or preventing an autosomal disorder in a subject identified as expressing a mutated gene variant, the method comprising introducing into a cell of the subject an effective amount of a composition comprising: a nuclease; and an exogenous nucleic acid encoding a knock-in cassette comprising a coding sequence for a wild-type gene and translation initiation and termination elements for expression of the wild-type gene; wherein the nuclease causes a break within a 5′ UTR of an endogenous nucleic acid encoding the mutated gene variant, wherein the endogenous nucleic acid encodes, in the 5′ to 3′ direction, the 5′ UTR, a translation initiation element for expression of the mutated gene variant, and a coding sequence for the mutated gene variant, wherein the nucleic acid encoding the knock-in cassette is integrated by homology-independent targeted integration into the 5′ UTR of the endogenous nucleic acid upstream (5′) of the translation initiation element encoded by the endogenous nucleic acid, and wherein integration of the nucleic acid encoding the knock-in cassette results in expression of the wild-type gene, and wherein expression of the wild-type gene results in decreased expression of the mutated gene variant. Aspect 82 is the method of Aspect 81, wherein the exogenous nucleic acid encoding the knock-in cassette is not integrated in-frame with the endogenous nucleic acid encoding the mutated gene variant. Aspect 83 is the method of Aspect 81, wherein the exogenous nucleic acid encoding the knock-in cassette is integrated in-frame with the endogenous nucleic acid encoding the mutated variant of the wild-type gene. Aspect 84 is the method of any one of Aspects 81-83, wherein the knock-in cassette is not flanked by homology arms. Aspect 85 is the method of any one of Aspects 81-84, wherein the nuclease is a CRISPR/Cas nuclease, and wherein the method further comprises contacting the cell with a guide molecule for the CRISPR/Cas nuclease. Aspect 86 is the method of any one of Aspects 81-85, wherein the nuclease is a zinc finger nuclease (ZFN), a transcription activator-like effector nuclease (TALEN), or a meganuclease. Aspect 87 is the method of any one of Aspects 81-86, wherein the nuclease is encoded by the same exogenous nucleic acid encoding the knock-in cassette, and wherein the exogenous nucleic acid is comprised in a vector. Aspect 88 is the method of Aspect 87, wherein the nuclease is a CRISPR/Cas nuclease, and wherein the vector comprising the exogenous nucleic acid encoding the nuclease and the knock-in cassette further comprises a nucleic acid encoding a guide molecule for the CRISPR/Cas nuclease. Aspect 89 is the method of Aspect 87 or Aspect 88, wherein the vector is a plasmid, a transposon, a cosmid, an artificial chromosome, a lipid nanoparticle, or a viral vector. Aspect 90 is the method of any one of Aspects 87-89, wherein the vector is a viral vector, and wherein the viral vector is an adeno-associated virus (AAV) vector, an adenovirus vector, a lentivirus vector, or a retrovirus vector. Aspect 91 is the method of Aspect 90, wherein the viral vector is an AAV vector. Aspect 92 is the method of any one of Aspects 81-91, wherein the nuclease is encoded by a nucleic acid that is different from the exogenous nucleic acid encoding the knock-in cassette, and wherein the nucleic acid encoding the nuclease and the exogenous nucleic acid encoding the knock-in cassette are comprised in two different vectors. Aspect 93 is the method of Aspect 92, wherein the nuclease is a CRISPR/Cas nuclease, and wherein the vector comprising the exogenous nucleic acid encoding the knock-in cassette further comprises a nucleic acid encoding a guide molecule for the CRISPR/Cas nuclease. Aspect 94 is the method of Aspect 92 or Aspect 93, wherein the vectors are a plasmid, a transposon, a cosmid, an artificial chromosome, a lipid nanoparticle, or a viral vector. Aspect 95 is the method of any one of Aspects 92-94, wherein the vectors are a viral vector, and wherein the viral vector is an adeno-associated virus (AAV) vector, an adenovirus vector, a lentivirus vector, or a retrovirus vector. Aspect 96 is the method of Aspect 95, wherein the viral vector is an AAV vector. Aspect 97 is the method of any one of Aspects 81-96, wherein the coding sequence for a wild-type gene is operably linked to a promoter. Aspect 98 is the method of Aspect 97, wherein the promoter is an inducible promoter, a constitutive promoter, or a tissue-specific promoter. Aspect 99 is the method of any one of Aspects 81-98, wherein the autosomal disorder is an autosomal dominant disorder. Aspect 100 is the method of Aspect 99, wherein the mutated gene variant is a dominant variant. Aspect 101 is the method of Aspect 99 or Aspect 100, wherein the wild-type gene is the RHO gene. Aspect 102 is the method of any one of Aspects 81-98, wherein the autosomal disorder is an autosomal recessive disorder. Aspect 103 is the method of Aspect 102, wherein the mutated gene variant is a recessive variant.

Aspect 104 is a composition comprising: a Cas9 nuclease; an exogenous nucleic acid encoding a knock-in cassette comprising a coding sequence for a wild-type gene and translation initiation and termination elements for expression of the wild-type gene; and a guide molecule for the CRISPR/Cas nuclease to direct the Cas9 to a 5′ UTR of an endogenous nucleic acid encoding a mutated variant of the wild-type gene, wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and wherein, when introduced into a cell, the nuclease causes a break within the 5′ UTR of the endogenous nucleic acid encoding the mutated variant of the wild-type gene, and wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid. Aspect 105 is the composition of Aspect 104, wherein the knock-in cassette is not flanked by homology arms. Aspect 106 is the composition of Aspect 104 or Aspect 105, wherein the Cas9 nuclease is encoded by a nucleic acid that is different from the exogenous nucleic acid encoding the knock-in cassette, and wherein the nucleic acid encoding the nuclease and the exogenous nucleic acid encoding the knock-in cassette are comprised in two different vectors. Aspect 107 is the composition of Aspect 106, wherein the vector comprising the exogenous nucleic acid encoding the knock-in cassette further comprises a nucleic acid encoding the guide molecule for the Cas9 nuclease. Aspect 108 is the composition of Aspect 106 or Aspect 107, wherein the vector is a plasmid, a transposon, a cosmid, an artificial chromosome, a lipid nanoparticle, or a viral vector. Aspect 109 is the composition of any one of Aspects 106-108, wherein the vector is a viral vector, and wherein the viral vector is an adeno-associated virus (AAV) vector, an adenovirus vector, a lentivirus vector, or a retrovirus vector. Aspect 110 is the composition of Aspect 109, wherein the viral vector is an AAV vector. Aspect 111 is the composition of any one of Aspects 106-110, wherein the coding sequence for the wild-type gene is operably linked to a promoter. Aspect 112 is the composition of Aspect 111, wherein the promoter is an inducible promoter, a constitutive promoter, or a tissue-specific promoter. Aspect 113 is the composition of any one of Aspects 106-112, wherein the mutated variant of the wild-type gene is a dominant variant. Aspect 114 is the composition of Aspect 113, wherein the wild-type gene is the RHO gene. Aspect 115 is the composition of any one of Aspects 106-112, wherein the mutated variant of the wild-type gene is a recessive variant. Aspect 116 is the composition of any one of Aspects 106-115, further comprising a pharmaceutically acceptable excipient.

Aspect 117 is a method comprising introducing into a subject a therapeutically effective amount of the composition of any one of Aspects 106-116. Aspect 118 is the method of Aspect 117, wherein the subject is a human or an animal. Aspect 119 is the method of Aspect 118, wherein the subject was previously identified as expressing a mutated variant of a wild-type gene. Aspect 120 is the method of Aspect 119, wherein introduction of a therapeutically effective amount of the composition of any one of Aspects 106-116 inhibits expression of the mutated variant of the wild-type gene. Aspect 121 is the method of any one of Aspects 117-120, wherein the mutated variant of the wild-type gene is a dominant variant. Aspect 122 is the method of any one of Aspects 117-121, wherein the wild-type gene is the RHO gene. Aspect 123 is the method of Aspect 122, wherein the composition is introduced to the retina of the subject. Aspect 124 is the method of any one of Aspects 117-120, wherein the mutated variant of the wild-type gene is a recessive variant.

Aspect 125 is a method of expressing a wild-type gene in cells, the method comprising introducing the composition of any one of Aspects 106-116 into the cells. Aspect 126 is the method of Aspect 125, wherein the cells are human or animal cells. Aspect 127 is the method of any one of Aspects 125-126, wherein the cells were previously determined to express a mutated variant of the wild-type gene. Aspect 128 is the method of Aspect 127, wherein expression of the wild-type gene inhibits expression of the mutated variant of the wild-type gene. Aspect 129 is the method of Aspect 127 or Aspect 128, wherein the mutated variant of the wild-type gene is a dominant variant. Aspect 130 is the method of Aspect 129, wherein the cells are retinal cells. Aspect 131 is the method of Aspect 130, wherein the wild-type gene is the RHO gene. Aspect 132 is the method of Aspect 127 or Aspect 128, wherein the mutated variant of the wild-type gene is a recessive variant.

Aspect 133 is a method of decreasing the expression of a mutated variant of a wild-type gene in cells, the method comprising introducing the composition of any one of Aspects 106-116 into the cells. Aspect 134 is the method of Aspect 133, wherein the cells are human or animal cells. Aspect 135 is the method of Aspect 133 or Aspect 134, wherein the mutated variant of the wild-type gene is a dominant variant. Aspect 136 is the method of Aspect 135, wherein the cells are retinal cells. Aspect 137 is the method of Aspect 136, wherein the wild-type gene is the RHO gene. Aspect 138 is the method of Aspect 133 or Aspect 134, wherein the mutated variant of the wild-type gene is a recessive variant

Aspect 139 is a method of increasing expression of wild-type RHO gene in the retina of a subject, the method comprising introducing into the retina of the subject a therapeutically effective amount of the composition of any one of Aspects 106-116. Aspect 140 is the method of Aspect 139, wherein the subject was previously identified as having retinal cells expressing a mutated variant of the RHO gene. Aspect 140 is the method of Aspect 140, wherein expression of the wild-type RHO gene inhibits expression of the mutated variant of the wild-type RHO gene. Aspect 142 is the method of any one of Aspects 139-141, wherein the subject is a human or an animal.

It is contemplated that any aspect discussed in this specification can be implemented with respect to any method or composition of the disclosure, and vice versa. Furthermore, compositions of the disclosure can be used to achieve methods of the disclosure.

Other objects, features, and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 schematically illustrates a homology-independent targeted integration (HITI) strategy for gene knock-in (KI) into the RHO 5′ UTR locus. Green pentagon, SpCas9-gRNA targeted region; Black line within pentagon, the cleaved site; Gray rectangle, exon. Yellow rectangle, Kozak sequence. Red rectangle, stop codon. Red asterisk, P23H mutation. HITI donor, the RHO coding sequence (CDS) flanked by two guide RNA (gRNA) targeting sequences.

FIG. 2 shows SpCas9 gRNA targeting sequences. Sequence in blue, the SpCas9 gRNA1/2 targeting sties; Sequence in red, PAM; Sequence highlighted by yellow, Kozak sequence; Sequence labeled in red, ATG start codon.

FIG. 3 schematically illustrates dual AAV vectors packaging SpCas9, mCherry, gRNA1, and a GFP- or RHO-HITI donor. SpCas9 and the mCherry reporter were driven by an hRK promoter. gRNA1 expression was driven by a U6 promoter.

FIG. 4 shows representative retina sections of the RHO−/− mice receiving GFP or RHO KI mediated by AAV8-SpCas9-gRNA1. Scale bar, 50 μm.

FIG. 5 shows quantification of the percentage of cells with KI (GFP+)/cells transduced by AAV (mCherry+) in retinal sections. Eyes were infected by hRK-SpCas9 gRNA1 (n=9) and hRK-SpCas9 GFP KI (n=12). Data are presented as mean±s.e.m. *P<0.05; **P<0.01; ***P<0.001; ****P<0.0001, unpaired two-tailed Student's t-test.

FIG. 6 shows next generation sequencing (NGS) results for allele frequency of RHO KI and insertions and/or deletions (INDELs) in the sorted mCherry+ photoreceptor cells.

FIG. 7 illustrates an experimental design for testing efficacy in the RHO^{P23H/wt} mice. Mouse eyes were untreated, treated with AAV8-SpCas9+AAV8-mCherry-U6-gRNA1 (labeled as SpCas9-gRNA1), or treated with AAV8-SpCas9+AAV8-mCherry-U6-gRNA1 RHO KI (labeled as SpCas9-Rho KI).

FIG. 8 shows representative optical coherent tomography (OCT) images showing the thickness of the outer nuclear layer (ONL). Measurements were performed at 0.6 mm from the optic nerve head (ONH) in the dorsal area every 30 days from P30 to P210.

FIG. 9 shows ONL thickness of RHO^{P23H/wt} with different treatments from P30 to P210. Data are presented as mean±s.e.m. *P<0.05; **P<0.01; ***P<0.001; ****P<0.0001, two-way ANOVA with Tukey post-hoc test.

FIG. 10 shows B wave amplitudes of rod scotopic electroretinography (ERG) responses of control and treated RHO^{P23H/wt} eyes under a light intensity of 0.032 cd·s·m⁻². Data are presented as mean±s.e.m. *P<0.05; **P<0.01; ***P<0.001; ****P<0.0001, two-way ANOVA with Tukey post-hoc test.

FIG. 11 shows B wave amplitudes of step-wise scotopic ERG responses of P180 RHO^{P23H/wt} eyes under a light intensity of −4.0 lg(cd·s·m⁻²) to 1.5 lg(cd·s·m⁻²). Data are presented as mean±s.e.m. *P<0.05; **P<0.01; ***P<0.001; ****P<0.0001, two-way ANOVA with Tukey post-hoc test.

FIG. 12 shows B wave amplitudes of mixed rod-cone ERG responses of P180 RHO^{P23H/wt} eyes under a light intensity of 30 cd·s·m⁻². Data are presented as mean±s.e.m. *P<0.05; **P<0.01; ***P<0.001; ****P<0.0001, two-way ANOVA with Tukey post-hoc test.

FIG. 13 shows representative retinal section images of RHO^{P23H/wt} mice at the endpoint P210. Sections were stained with anti-mCAR (cone marker, in white) and anti-RHO (rod marker, in green) antibodies. Scale bar in the left column showing whole retinal sections: 500 μm. Scale bar in other columns: 50 μm.

FIGS. 14-15 show ONL thickness quantification of P210 RHO^{P23H/wt} retinas at different distances from the ONH. Untreated group (n=14); SpCas9-gRNA1 (n=5); SpCas9-RHO KI (n=9). Data are presented as mean±s.e.m. *P<0.05; **P<0.01; ***P<0.001; ****P<0.0001, two-way ANOVA with Tukey post-hoc test.

FIGS. 16A-16C show in vitro screening of SpCas9 gRNAs to target the RHO 5′UTR. FIG. 16A. Schematic of the expression plasmid for screening spCas9 gRNAs targeting efficiency. CMV and U6 are promoters to drive transcription of Cas9, mCherry, and gRNA, respectively. BGHpA, Bovine Growth Hormone Polyadenylation. 2A, self-cleaving peptide sequence. FIG. 16B. Timeline of Cas9-gRNA targeting efficiency evaluation. The Cas9-gRNA plasmids were transfected into wide-type MEF cells, and the mCherry+ cells were sorted after 3 days of transfection for genomic DNA extraction and gene editing analysis. FIG. 16C. The targeting efficiency of the SpCas9-gRNAs was analyzed by ICE CRISPR Analysis tools. SpCas9-gRNA1 showed the highest knock-out efficiency, 41%.

FIG. 17 shows viral infection and gene integration efficiency into the RHO locus mediated by AAV-Cas9-gRNA. Representative FACS plots of dissociated cells from retinas that were untreated (left panel) or infected by AAV8-gRNA-GFP donor only (middle panel) or infected by AAV8-Cas9 and AAV8-gRNA-GFP donor (right panel).

FIGS. 18A-18B schematically illustrate RHO or GFP integration in RHO −/− mice. FIG. 18A. Schematic of AAV vectors delivering SpCas9, gRNA, and RHO or GFP donor to the retina. hRK promoter controls expression of SpCas9 and mCherry. gRNA1 was controlled under the U6 promoter. The donor was flanked by 2 targeting sites of SpCas9-gRNA. In RHO KO transgenic mice, the RHO exon1 was replaced by a PGK Neo cassette; however, the SpCas9-gRNA targeting site was not changed. FIG. 18B. Experimental design of CRISPR/Cas9 mediated RHO or GFP integration in RHO−/− mice.

FIGS. 19A-19C show gene integration in the 5′ UTR disrupts the endogenous RHO expression. FIG. 19A. Schematic representation of gene knock-in into the RHO locus mediated by SpCas9-gRNA1. Green arrow, endogenous promotor of RHO. Yellow rectangles, Kozak sequence. Blue rectangles, exogenous RHO or GFP knock-in sequence. Green polygons, reverse complementary sequence induced by HITI-mediated gene knock-in. Gray rectangles, endogenous RHO coding exons. Red rectangles, stop codon. FIG. 19B. Mimic 5′ UTR gene knock-in sequence. Left: plasmid containing the product of gene integration into RHO locus mediated by SpCas9-gRNA1, CMV-Kozak-GFP-Kozak-RHO. Right: control plasmid CMV-Kozak-RHO-Kozak-GFP. These plasmids were transfected into 293T cells to determine if the downstream gene was expressed. FIG. 19C. Representative images of 293T cells transfected by Kozak-GFP-Kozak-RHO or Kozak-RHO-Kozak-GFP plasmid. Only the first gene in those cassettes could be expressed. Scale bar, 50 μm.

FIGS. 20A-20E show the effect of 5′ UTR genomic modification on visual function and RHO expression. FIG. 20A. Schematic representation of RHO CDS modification and RHO 5′ UTR modification. Green pentagon, SaCas9 or SpCas9-gRNA targeted regions; Black line within pentagon, cleaved site; Gray rectangle, exon. Yellow rectangle, Kozak sequence. Red rectangle, stop codon sequence. FIG. 20B. RHO expression level in purified rods from the eyes of wild-type mice injected with AAV8-gRNA2 only (n=3) and AAV8-SaCas9-gRNA1 (n=3) or AAV8-gRNA1 only (n=3) and AAV8-SpCas9-gRNA1 (n=3). FIG. 20C. P30 rod scotopic ERG response of wild-type mice that were untreated (n=10) or treated with AAV8-gRNA1 only (n=5) or AAV8-SpCas9-gRNA1 (n=5). FIG. 20D. P30 cone photopic ERG response of wild-type mice. FIG. 20E. P30 mixed rod and cone scotopic ERG response of wild-type mice. For the ERG measurement, only B-wave data are shown. Data are presented as mean±s.e.m, unpaired two-tailed Student's t-test (FIG. 19B), one-way ANOVA with Tukey post-hoc test (FIGS. 19C-19E).

FIG. 21 shows RHO integration mediated by AAV-SpCas9 conserved cone function of RHO^{P23H/wt} mice. P30-P210 cone photopic ERG responses of RHO^{P23H/wt} mice eyes that were untreated (n=14), or treated with AAV8-SpCas9-gRNA1 (n=5), or AAV8-SpCas9-RHO KI (n=9) under a light intensity of 30 cd·s·m⁻² after 10 cd·s·m⁻² light adaptation. Each dot represents a B-wave amplitude. The box and whiskers show mean±s.e.m. *P<0.05, two-way ANOVA with Tukey post-hoc test.

FIGS. 22A-22B show PAM sites in adRP gene 5′ UTRs possess high cross-species conservation and easy accessibility. FIG. 22A. RHO genomic DNA alignment between Mus musculus, Macaca fascicular, and Homo sapiens. FIG. 22B. Potential SpCas9 target sites in 5′ UTR of other human adRP associated genes. Sequence in blue, SpCas9-gRNA target regions. Asterisk, the same nucleobase. Sequence in orange, PAM site. Region highlighted by yellow, Kozak sequence.

FIGS. 23-28 schematically illustrate the various plasmids used for gene knock-in therapy.

### DETAILED DESCRIPTION

This disclosure is based, at least in part, on the development of novel 5′ untranslated region (UTR)-targeting gene knock-in (KI) compositions and methods of use are disclosed herein. The gene KI compositions and methods exploit homology-independent targeted integration (HITI)-mediated insertion of a wild-type coding sequence (CDS) into the 5′ untranslated region (UTR) upstream of a translation initiation element of a mutated variant of the wild-type gene. The experimental results included below demonstrate the surprising safety and efficiency of the disclosed 5′ UTR-targeting gene KI therapy compositions and methods compared to other gene therapy approaches.

Despite the recent success of gene supplementation therapy for monogenic recessive diseases, therapeutic approaches to treat autosomal dominant disorders are still needed. Accordingly, disclosed herein, in some aspects, is a new gene knock-in (KI) therapy that exploits AAV-Cas9-mediated HITI of a wild-type CDS into the 5′ UTR, more specifically immediately upstream of a translation initiation element (e.g., a Kozak sequence), of a mutated variant of the wild-type gene. As described in the examples herein, this approach was tested in the heterozygous RHO^{P23H/wt} mice, which carry the most common dominant point mutation found in autosomal dominant Retinitis Pigmentosa (adRP) patients. In some aspects, the HITI-AAVs mediate highly efficient gene insertion in the mouse RHO 5′ UTR in vivo. In some aspects, the HITI-AAVs significantly prolonged photoreceptor survival and visual function.

The mutation-independent gene KI therapy approach disclosed herein that targets 5′ UTR of mutated variants of wild-type genes demonstrates therapeutic potential to treat, e.g., autosomal disorders, and demonstrates improvements over other AAV-HITI-mediated gene KI approaches at least because, in some aspects, the present compositions and methods provide at least the following advantages: 1) the 5′ UTR KI approach is not mutation-specific; 2) the inserted wild-type gene CDS is under the control of the cis-regulatory sequence in the genomic context; 3) 5′UTR KI has higher insertion efficiency, as the inserted sequence can but is not required to be in-frame with the endogenous CDS; 4) expression of truncated proteins from the endogenous allele is inhibited, thereby reducing or avoiding possible toxic dominant-negative effects; and 5) insertions and/or deletions (INDELs) in the 5′ UTR does not eliminate the expression of wild type allele, but INDELs in the CDS may lead to a reading frame shift and a knock out effect on the wild type allele.

Accordingly, provided herein, in some aspects, are methods and compositions for editing the genome of a cell and engineered cells produced using such compositions and methods. In some aspects, the cell can be contacted with a composition comprising a nuclease and an exogenous nucleic acid encoding a knock-in cassette comprising a coding sequence for a wild-type gene and translation initiation and termination elements for expression of the wild-type gene. The nuclease may cause a break within a 5′ UTR of an endogenous nucleic acid encoding a mutated variant of the wild-type gene, which may encode, in the 5′ to 3′ direction, the 5′ UTR, a translation initiation element for expression of the mutated variant of the wild-type gene, and a coding sequence for the mutated variant of the wild-type gene. The exogenous nucleic acid encoding the knock-in cassette can be integrated by homology-independent targeted integration into the 5′ UTR of the endogenous nucleic acid upstream (5′) of the translation initiation element encoded by the endogenous nucleic acid. Integration of the exogenous nucleic acid encoding the knock-in cassette can result in expression of the wild-type gene by the cell, and expression of the wild-type gene by the cell can inhibit expression of the mutated variant of the wild-type gene. Also disclosed, in some aspects, are methods of expressing a wild-type gene (e.g., a wild-type gene from which a mutated gene variant is derived, e.g., RHO) in cells, methods of decreasing the expression of a mutated variant of a wild-type gene (e.g., a wild-type gene from which a mutated gene variant is derived, e.g., RHO) in cells, methods for treating or preventing autosomal dominant disorders (e.g., retinitis pigmentosa) in subjects, and methods for treating or preventing autosomal recessive disorders.

### I. Examples of Definitions

Reference throughout this specification to “one aspect,” “an aspect,” “a particular aspect,” “a related aspect,” “a certain aspect,” “an additional aspect,” or “a further aspect” or combinations thereof means that a particular feature, structure, or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

Throughout this application, the term “about” is used to indicate that a value includes the inherent variation of error (e.g., a deviation of ±10%) for the measurement or quantitation method.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The use of the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to whatever follows the phrase “consisting of.” Thus, the phrase “consisting of” indicates that the listed elements are required or mandatory, and that no other elements may be present. Compositions and methods “consisting essentially of” any of the ingredients or steps disclosed limits the scope of the claim to any elements listed after the phrase and the specified materials or steps which do not materially affect the basic and novel characteristic of the aspects of the disclosure. Thus, the phrase “consisting essentially of” indicates that the listed elements are required or mandatory, but that other elements are optional and may or may not be present depending upon whether or not they affect the activity or action of the listed elements.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “endogenous” refers to any material originating from within an organism, cell, or tissue.

The term “exogenous” refers to any material introduced from or originating from outside an organism, cell, or tissue that is not produced or does not originate from the same organism, cell, or tissue in which it is being introduced.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The terms “transfected,” “transformed,” or “transduced” or any variation of these terms refer to a process by which exogenous nucleic acid is transferred or introduced into a cell. A “transfected,” “transformed,” or “transduced” cell is one that has been transfected, transformed or transduced with exogenous nucleic acid.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, and complementary sequences of the foregoing described herein.

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

The term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

The term “expression” refers to the generation of any gene product from the nucleic acid sequence. In some aspects, a gene product may be a transcript. In some aspects, a gene product may be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

The terms “protein,” “polypeptide,” or “peptide” are used herein as synonyms and refer to a polymer of amino acid monomers, e.g., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the terms “wild-type” or “wildtype” or “WT” or “native” refer to the endogenous version of a molecule that occurs naturally in an organism. In some aspects, a wild-type polypeptide or nucleic acid sequence has a sequence that has not been intentionally modified. In some aspects, wild-type versions of a polynucleotide or polypeptide are employed, however, other aspects of the disclosure relate to a modified or variant polynucleotide or polypeptide. A “modified” polynucleotide or polypeptide or a “variant” polynucleotide or polypeptide refers to a polynucleotide or polypeptide whose chemical structure, particularly its nucleotide or amino acid sequence, is altered with respect to the wild-type polynucleotide or polypeptide. In some aspects, a modified/variant polynucleotide or polypeptide has at least one modified activity or function (recognizing that polynucleotides or polypeptides may have multiple activities or functions). Where a polynucleotide or polypeptide is specifically mentioned herein, it is in general a reference to a native (wild-type) or recombinant (modified/variant) polynucleotide or polypeptide. The polynucleotide or polypeptide may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., a wild-type gene coding sequence). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In general, whether a particular molecule is properly considered to be a “variant” of a reference molecule (e.g., a wild-type molecule) is based on its degree of structural identity with the reference molecule (e.g., the wild-type molecule). As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone).

In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid.

In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. In some aspects, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 25, about 20, about 19, about 18, about 17, about 16, about 15, about 14, about 13, about 10, about 9, about 8, about 7, about 6, about 5, about 4, about 3, or about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, about 5, about 4, about 3, or about 2 additions or deletions as compared to the reference.

For the purposes of the present disclosure, “variants” of an amino acid sequence (peptide, protein, or polypeptide) comprise amino acid insertion variants, amino acid addition variants, amino acid deletion variants and/or amino acid substitution variants. “Variants” of a nucleotide sequence comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring.

In the present disclosure, a “vector” refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, and transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “subject,” as used herein, can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as an autosomal disease or disorder (i.e., an autosomal dominant disorder or an autosomal recessive disorder). The subject may be undergoing or having undergone treatment. The subject may be asymptomatic. The subject may be healthy individuals but that are desirous of prevention of an autosomal disease or disorder (i.e., an autosomal dominant disorder or an autosomal recessive disorder). The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies. In specific aspects, the subject is a human. In specific aspects, the subject is an animal.

As used herein “treatment” or “treating,” includes any beneficial or desirable effect on the symptoms or pathology of a disease or pathological condition and may include even minimal reductions in one or more measurable markers of the disease or condition being treated, e.g., autosomal diseases or disorders. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood or risk of the occurrence or recurrence of, a disease or condition, e.g., an autosomal disease. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

A treatment is “therapeutically effective” when it results in a reduction in one or more of the number, severity, and frequency of one or more symptoms of a disease state (e.g., an autosomal disorder) in a subject (e.g., a human or an animal). In some aspects, a therapeutically effective amount of a composition can result in an increase in the expression level of an active wild-type protein (e.g., a wildtype RHO protein or of a variant of a RHO protein that has the desired activity) (e.g., as compared to the expression level prior to treatment with the composition). In some aspects, a therapeutically effective amount of a composition can result in an increase in the expression level of an active wild-type protein (e.g., a wildtype RHO protein or active variant) in a target cell (e.g., a retinal cell). In some aspects, a therapeutically effective amount of a composition can result in an increase in the expression level of an active wild-type protein (e.g., a wild-type RHO protein or active variant), and/or an increase in one or more activities of a wild-type protein in a target cell (e.g., as compared to a reference level, such as the level(s) in a subject prior to treatment, the level(s) in a subject having a mutation in a wild-type gene, or the level(s) in a subject or a population of subjects having an autosomal disorder).

The phrase “pharmaceutically acceptable” includes compositions that do not produce an allergic or similar untoward reaction when administered to a human or an animal. Typically, such compositions are prepared either as topical compositions, liquid solutions or suspensions, solid forms suitable for solution in, or suspension in, liquid prior to use can also be prepared. Routes of administration can vary with the location and nature of the condition to be treated, and include, e.g., topical, inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation.

### II. Autosomal Disorders

Provided herein, in some aspects, are methods and compositions for treating or preventing autosomal disorders in subjects identified as expressing a mutated gene variant. Cells expressing endogenous mutated variants of wild-type genes (i.e., mutated gene variants) can be targeted for the purpose of improving an autosomal disorder in an individual that has the autosomal disorder or for the purpose of reducing the risk or delaying the severity and/or onset of the autosomal disorder in an individual. In specific cases, cells expressing endogenous mutated variants of wild-type genes (i.e., mutated gene variants) that cause, at least in part, the autosomal disorder are targeted for the purpose of inhibiting or reducing expression of the endogenous mutated variants of wild-type genes.

A. Autosomal Dominant Disorders

In some aspects, the autosomal disorder to be treated or prevented by the methods and/or compositions disclosed herein is an autosomal dominant disorder. Therefore, in some aspects, the mutated variant of the wild-type gene is a dominant variant, and cells expressing dominant variants are targeted for the purpose of inhibiting or reducing expression of the dominant variants of wild-type genes.

Autosomal dominant is a pattern of inheritance characteristic of some genetic disorders. “Autosomal” means that the gene in question is located on one of the numbered, or non-sex, chromosomes. “Dominant” means that a single copy of the mutated gene (from one parent) is enough to cause the disorder. In other words, dominance is the phenomenon of one variant (allele) of a gene on a chromosome (i.e., the dominant variant) masking or overriding the effect of a different variant of the same gene on the other copy of the chromosome (i.e., the recessive variant).

A child of a person affected by an autosomal dominant condition has a 50% chance of being affected by that condition via inheritance of a dominant allele. To illustrate, if one parent is affected with an autosomal dominant disorder, thus heterozygous (Aa), while the other parent is not affected and homozygous (aa), then 50% of the offspring will have the chance of 1) receiving one dominant allele, resulting in the heterozygous (Aa) state and being affected with the disorder or 2) receiving both recessive alleles, resulting in the homozygous (aa) state and not being affected with the disorder. If both parents are heterozygous and affected by the disorder, 75% of the offspring have the chance of inheriting a dominant allele and being affected by the disorder. If one parent is homozygous (i.e., AA) and affected by the disorder, all of the offspring may have the chance of inheriting a dominant allele and being affected by the disorder. By contrast, an autosomal recessive disorder requires two copies of the mutated gene (one from each parent) to cause the disorder. Autosomal dominant disorders involve autosomes or the non-sex chromosomes and can therefore affect males and females equally.

Mutations in genes associated with autosomal dominant (AD) conditions are known to result in either loss or gain of function. In some aspects, loss-of-function mutations are distributed uniformly along protein sequence, while gain-of-function mutations are localized to key regions. Though dominant inheritance is common, dominant conditions can occur sporadically or de novo within a family due to sporadic mutation in parental gonads or within the developing fetus. In fact, many autosomal dominant disorder mutations arise de novo, or for the first time within a family, in an affected individual. These de novo mutations are not inherited from a parent.

Autosomal dominant disorders can be characterized by their penetrance, measured by the percentage of individuals who inherit a disorder allele and display the phenotype of the disorder. All individuals who inherit a disorder allele may not exhibit the phenotype of the disorder (reduced penetrance); however, they can still pass on the allele and have an affected child. Autosomal dominant disorders with a higher penetrance result in more individuals displaying the phenotype who inherit the disorder allele. Furthermore, when age is a factor for the disorder (i.e., it only is apparent in adulthood), the family history may not appear to be dominant if the individuals you are assessing are not yet old enough to display the phenotype. Early identification of autosomal dominant disease can be important for reducing morbidity and mortality.

Examples of autosomal dominant disorders that may be treated or prevented according to the compositions and methods of the present disclosure include, but art not limited to, achondroplasia, acute intermittent porphyria, antithrombin III deficiency, BRCA1/BRCA2 positive breast cancer, cherubism, dominant blindness (e.g., Leber congenital amaurosis, retinitis pigmentosa, Stargardt-like macular dystrophy, stationary night blindness, vitreoretinochoroidopathy), dominant congenital deafness, Ehlers-Danlos syndrome, familial adenomatous polyposis, Gilbert's disease, hereditary hemorrhagic telangiectasia, hereditary elliptosis, hereditary spherocytosis, holoproencephaly, Huntington's disease, hypercholesterolemia, idiopathic hypoparathyroidism, intestinal polyposis, marble bone disease, Marfan's syndrome, myotonic dystrophy, neurofibromatosis, osteogenesis imperfecta, polycystic kidney disease, protein C deficiency, retinitis pigmentosa, retinoblastoma, Treacher Collins syndrome, tuberous sclerosis, and Von Willebrand's disease.

1. Retinitis Pigmentosa

In specific aspects, the autosomal disorder is an autosomal dominant disorder. In specific aspects, the autosomal dominant disorder is a dominant blindness disorder. In specific aspects, the autosomal dominant disorder is a dominant blindness disorder, and the dominant blindness disorder is retinitis pigmentosa. Retinitis pigmentosa is a group of related eye disorders that cause progressive vision loss. These disorders affect the retina, which is the layer of light-sensitive tissue at the back of the eye. Retinitis pigmentosa is one of the most common inherited diseases of the retina (retinopathies). It is estimated to affect 1 in 3,500 to 1 in 4,000 people in the United States and Europe. In some aspects, treating or preventing retinitis pigmentosa comprises treatment or prevention of one or more symptoms of retinitis pigmentosa. In people with retinitis pigmentosa, vision loss occurs as the light-sensing cells of the retina gradually deteriorate. The first sign of retinitis pigmentosa is usually a loss of night vision, which becomes apparent in childhood. Problems with night vision can make it difficult to navigate in low light. Later, the disease causes blind spots to develop in the side (peripheral) vision. Over time, these blind spots merge to produce tunnel vision. The disease progresses over years or decades to affect central vision, which is needed for detailed tasks such as reading, driving, and recognizing faces. In adulthood, many people with retinitis pigmentosa become legally blind. The signs and symptoms of retinitis pigmentosa are most often limited to vision loss. When the disorder occurs by itself, it is described as nonsyndromic.

The genes associated with retinitis pigmentosa play essential roles in the structure and function of specialized light receptor cells (photoreceptors) in the retina. The retina contains two types of photoreceptors, rods and cones. Rods are responsible for vision in low light, while cones provide vision in bright light, including color vision. Mutations in any of the genes responsible for retinitis pigmentosa lead to a gradual loss of rods and cones in the retina. The progressive degeneration of these cells causes the characteristic pattern of vision loss that occurs in people with retinitis pigmentosa. Rods typically break down before cones, which is why night vision impairment is usually the first sign of the disorder. Daytime vision is disrupted later, as both rods and cones are lost.

The nucleotide as well as the protein, polypeptide, and peptide sequences for various genes associated with retinitis pigmentosa that may be selected for or excluded from gene KI therapy according to the compositions and methods of the disclosure include ABCA4, BEST1, C2ORF71, C8ORF37, CLRN, CRB1, CRX, PDE6B, PRPH2, RHO, RP2, RPE65, RPGR, USH2A, WDR19, CA4, CERKL, CNGA1, CNGB1, DHDDS, EYS, FAM161A, FSCN2, GUCA1B, IDH3B, IMPDH, IMPG2, KLHL7, LRAT, MAK, MERTK, MT-TS2, NR2E3, NRL, OFD1, PCARE, PDE6A, PDE6G, PRCD, PROM1, PRPF3, PRPF31, PRPF8, PRPF31, RBP3, RDH12, RGR, RLBP1, ROM1, RP1, RP9, SAG, SEMA4A, SNRNP200, SPATA7, TOPORS, TTC8, TULP1, and ZNF513. Mutated variants of any one or more of these genes may be selected for or excluded from gene KI therapy according to the compositions and methods of the disclosure.

In specific aspects, the endogenous RHO gene is mutated from wild-type and is selected for gene KI therapy according to the compositions and methods of the disclosure. Mutated variant RHO gene coding sequences may be derived from the wild-type human RHO gene nucleotide and amino acid coding sequences provided by NCBI Gene ID: 6010, corresponding to GenBank Accession No. AB065668, incorporated by reference herein in their entirety and reproduced below as SEQ ID NOs:1 and 2:

(SEQ ID NO: 1)
GGGGATTAATATGATTATGAACACCCCCAATCTCCCAGATGCTGATTC
AGCCAGGAGCTTAGGAGGGGGAGGTCACTTTATAAGGGTCTGGGGGGGTCAGAA
CCCAGAGTCATCCAGCTGGAGCCCTGAGTGGCTGAGCTCAGGCCTTCGCAGCATT
CTTGGGTGGGAGCAGCCACGGGTCAGCCACAAGGGCCACAGCCATGAATGGCAC
AGAAGGCCCTAACTTCTACGTGCCCTTCTCCAATGCGACGGGTGTGGTACGCAGC
CCCTTCGAGTACCCACAGTACTACCTGGCTGAGCCATGGCAGTTCTCCATGCTGG
CCGCCTACATGTTTCTGCTGATCGTGCTGGGCTTCCCCATCAACTTCCTCACGCTC
TACGTCACCGTCCAGCACAAGAAGCTGCGCACGCCTCTCAACTACATCCTGCTCA
ACCTAGCCGTGGCTGACCTCTTCATGGTCCTAGGTGGCTTCACCAGCACCCTCTA
CACCTCTCTGCATGGATACTTCGTCTTCGGGCCCACAGGATGCAATTTGGAGGGC
TTCTTTGCCACCCTGGGCGGTATGAGCCGGGTGTGGGTGGGGTGTGCAGGAGCCC
GGGAGCATGGAGGGGTCTGGGAGAGTCCCGGGCTTGGCGGTGGTGGCTGAGAGG
CCTTCTCCCTTCTCCTGTCCTGTCAATGTTATCCAAAGCCCTCATATATTCAGTCA
ACAAACACCATTCATGGTGATAGCCGGGCTGCTGTTTGTGCAGGGCTGGCACTGA
ACACTGCCTTGATCTTATTTGGAGCAATATGCGCTTGTCTAATTTCACAGCAAGA
AAACTGAGCTGAGGCTCAAAGAAGTCAAGCGCCCTGCTGGGGCGTCACACAGGG
ACGGGTGCAGAGTTGAGTTGGAAGCCCGCATCTATCTCGGGCCATGTTTGCAGCA
CCAAGCCTCTGTTTCCCTTGGAGCAGCTGTGCTGAGTCAGACCCAGGCTGGGCAC
TGAGGGAGAGCTGGGCAAGCCAGACCCCTCCTCTCTGGGGGCCCAAGCTCAGGG
TGGGAAGTGGATTTTCCATTCTCCAGTCATTGGGTCTTCCCTGTGCTGGGCAATG
GGCTCGGTCCCCTCTGGCATCCTCTGCCTCCCCTCTCAGCCCCTGTCCTCAGGTGC
CCCTCCAGCCTCCCTGCCGCGTTCCAAGTCTCCTGGTGTTGAGAACCGCAAGCAG
CCGCTCTGAAGCAGTTCCTTTTTGCTTTAGAATAATGTCTTGCATTTAACAGGAAA
ACAGATGGGGTGCTGCAGGGATAACAGATCCCACTTAACAGAGAGGAAAACTGA
GGCAGGGAGAGGGGAAGAGACTCATTTAGGGATGTGGCCAGGCAGCAACAAGA
GCCTAGGTCTCCTGGCTGTGATCCAGGAATATCTCTGCTGAGATGCAGGAGGAGA
CGCTAGAAGCAGCCATTGCAAAGCTGGGTGACGGGGAGAGCTTACCGCCAGCCA
CAAGCGTCTCTCTGCCAGCCTTGCCCTGTCTCCCCCATGTCCAGGCTGCTGCCTCG
GTCCCATTCTCAGGGAATCTCTGGCCATTGTTGGGTGTTTGTTGCATTCAATAATC
ACAGATCACTCAGTTCTGGCCAGAAGGTGGGTGTGCCACTTACGGGTGGTTGTTC
TCTGCAGGGTCAGTCCCAGTTTACAAATATTGTCCCTTTCACTGTTAGGAATGTCC
CAGTTTGGTTGATTAACTATATGGCCACTCTCCCTATGGAACTTCATGGGGTGGT
GAGCAGGACAGATGTCTGAATTCCATCATTTCCTTCTTCTTCCTCTGGGCAAAAC
ATTGCACATTGCTTCATGGCTCCTAGGAGAGGCCCCCACATGTCCGGGTTATTTC
ATTTCCCGAGAAGGGAGAGGGAGGAAGGACTGCCAATTCTGGGTTTCCACCACC
TCTGCATTCCTTCCCAACAAGGAACTCTGCCCCACATTAGGATGCATTCTTCTGCT
AAACACACACACACACACACACACACACAACACACACACACACACACACACACA
CACACACACAAAACTCCCTACCGGGTTCCCAGTTCAATCCTGACCCCCTGATCTG
ATTCGTGTCCCTTATGGGCCCAGAGCGCTAAGCAAATAACTTCCCCCATTCCCTG
GAATTTCTTTGCCCAGCTCTCCTCAGCGTGTGGTCCCTCTGCCCCTTCCCCCTCCT
CCCAGCACCAAGCTCTCTCCTTCCCCAAGGCCTCCTCAAATCCCTCTCCCACTCCT
GGTTGCCTTCCTAGCTACCCTCTCCCTGTCTAGGGGGGAGTGCACCCTCCTTAGG
CAGTGGGGTCTGTGCTGACCGCCTGCTGACTGCCTTGCAGGTGAAATTGCCCTGT
GGTCCTTGGTGGTCCTGGCCATCGAGCGGTACGTGGTGGTGTGTAAGCCCATGAG
CAACTTCCGCTTCGGGGAGAACCATGCCATCATGGGCGTTGCCTTCACCTGGGTC
ATGGCGCTGGCCTGCGCCGCACCCCCACTCGCCGGCTGGTCCAGGTAATGGCACT
GAGCAGAAGGGAAGAAGCTCCGGGGGCTCTTTGTAGGGTCCTCCAGTCAGGACT
CAAACCCAGTAGTGTCTGGTTCCAGGCACTGACCTTGTATGTCTCCTGGCCCAAA
TGCCCACTCAGGGTAGGGGTGTAGGGCAGAAGAAGAAACAGACTCTAATGTTGC
TACAAGGGCTGGTCCCATCTCCTGAGCCCCATGTCAAACAGAATCCAAGACATCC
CAACCCTTCACCTTGGCTGTGCCCCTAATCCTCAACTAAGCTAGGCGCAAATTCC
AATCCTCTTTGGTCTAGTACCCCGGGGGCAGCCCCCTCTAACCTTGGGCCTCAGC
AGCAGGGGAGGCCACACCTTCCTAGTGCAGGTGGCCATATTGTGGCCCCTTGGA
ACTGGGTCCCACTCAGCCTCTAGGCGATTGTCTCCTAATGGGGCTGAGATGAGAC
ACAGTGGGGACAGTGGTTTGGACAATAGGACTGGTGACTCTGGTCCCCAGAGGC
CTCATGTCCCTCTGTCTCCAGAAAATTCCCACTCTCACTTCCCTTTCCTCCTCAGT
CTTGCTAGGGTCCATTTCTTACCCCTTGCTGAATTTGAGCCCACCCCCTGGACTTT
TTCCCCATCTTCTCCAATCTGGCCTAGTTCTATCCTCTGGAAGCAGAGCCGCTGGA
CGCTCTGGGTTTCCTGAGGCCCGTCCACTGTCACCAATATCAGGAACCATTGCCA
CGTCCTAATGACGTGCGCTGGAAGCCTCTAGTTTCCAGAAGCTGCACAAAGATCC
CTTAGATACTCTGTGTGTCCATCTTTGGCCTGGAAAATACTCTCACCCTGGGGCTA
GGAAGACCTCGGTTTGTACAAACTTCCTCAAATGCAGAGCCTGAGGGCTCTCCCC
ACCTCCTCACCAACCCTCTGCGTGGCATAGCCCTAGCCTCAGCGGGCAGTGGATG
CTGGGGCTGGGCATGCAGGGAGAGGCTGGGTGGTGTCATCTGGTAACGCAGCCA
CCAAACAATGAAGCGACACTGATTCCACAAGGTGCATCTGCATCCCCATCTGATC
CATTCCATCCTGTCACCCAGCCATGCAGACGTTTATGATCCCCTTTTCCAGGGAG
GGAATGTGAAGCCCCAGAAAGGGCCAGCGCTCGGCAGCCACCTTGGCTGTTCCC
AAGTCCCTCACAGGCAGGGTCTCCCTACCTGCCTGTCCTCAGGTACATCCCCGAG
GGCCTGCAGTGCTCGTGTGGAATCGACTACTACACGCTCAAGCCGGAGGTCAAC
AACGAGTCTTTTGTCATCTACATGTTCGTGGTCCACTTCACCATCCCCATGATTAT
CATCTTTTTCTGCTATGGGCAGCTCGTCTTCACCGTCAAGGAGGTACGGGCCGGG
GGGTGGGCGGCCTCACGGCTCTGAGGGTCCAGCCCCCAGCATGCATCTGCGGCTC
CTGCTCCCTGGAGGAGCCATGGTCTGGACCCGGGTCCCGTGTCCTGCAGGCCGCT
GCCCAGCAGCAGGAGTCAGCCACCACACAGAAGGCAGAGAAGGAGGTCACCCG
CATGGTCATCATCATGGTCATCGCTTTCCTGATCTGCTGGGTGCCCTACGCCAGC
GTGGCATTCTACATCTTCACCCACCAGGGCTCCAACTTCGGTCCCATCTTCATGAC
CATCCCAGCGTTCTTTGCCAAGAGCGCCGCCATCTACAACCCTGTCATCTATATC
ATGATGAACAAGCAGGTGCCTACTGCGGGTGGGAGGGCCCCAGTGCCCCAGGCC
ACAGGCGCTGCCTGCCAAGGACAAGCTACTTCCCAGGGCAGGGGAGGGGGCTCC
ATCAGGGTTACTGGCAGCAGTCTTGGGTCAGCAGTCCCAATGGGGAGTGTGTGA
GAAATGCAGATTCCTGGCCCCACTCAGAACTGCTGAATCTCAGGGTGGGCCCAG
GAACCTGCATTTCCAGCAAGCCCTCCACAGGTGGCTCAGATGCTCACTCAGGTGG
GAGAAGCTCCAGTCAGCTAGTTCTGGAAGCCCAATGTCAAAGTCAGAAGGACCC
AAGTCGGGAATGGGATGGGCCAGTCTCCATAAAGCTGAATAAGGAGCTAAAAAG
TCTTATTCTGAGGGGTAAAGGGGTAAAGGGTTCCTCGGAGAGGTACCTCCGAGG
GGTAAACAGTTGGGTAAACAGTCTCTGAAGTCAGCTCTGCCATTTTCTAGCTGTA
TGGCCCTGGGCAAGTCAATTTCCTTCTCTGTGCTTTGGTTTCCTCATCCATAGAAA
GGTAGAAAGGGCAAAACACCAAACTCTTGGATTACAAGAGATAATTTACAGAAC
ACCCTTGGCACACAGAGGGCACCATGAAATGTCACGGGTGACACAGCCCCCTTG
TGCTCAGTCCCTGGCATCTCTAGGGGTGAGGAGCGTCTGCCTAGCAGGTTCCCTC
CAGGAAGCTGGATTTGAGTGGATGGGGCGCTGGAATCGTGAGGGGCAGAAGCAG
GCAAAGGGTCGGGGCGAACCTCACTAACGTGCCAGTTCCAAGCACACTGTGGGC
AGCCCTGGCCCTGACTCAAGCCTCTTGCCTTCCAGTTCCGGAACTGCATGCTCAC
CACCATCTGCTGCGGCAAGAACCCACTGGGTGACGATGAGGCCTCTGCTACCGTG
TCCAAGACGGAGACGAGCCAGGTGGCCCCGGCCTAAGACCTGCCTAGGACTCTG
TGGCCGACTATAGGCGTCTCCCATCCCCTACACCTTCCCCCAGCCACAGCCATCC
CACCAGGAGCAGCGCCTGTGCAGAATGAACGAAGTCACATAGGCTCCTTAATTTT
TTTTTTTTTTTTAAGAAATAATTAATGAGGCTCCTCACTCACCTGGGACAGCCTGA
GAAGGGACATCCACCA
(SEQ ID NO: 2)
MNGTEGPNFYVPFSNATGVVRSPFEYPQYYLAEPWQFSMLAAYMFLLIVL
GFPINFLTLYVTVQHKKLRTPLNYILLNLAVADLFMVLGGFTSTLYTSLHGYFVFGPT
GCNLEGFFATLGGEIALWSLVVLAIERYVVVCKPMSNFRFGENHAIMGVAFTWVMA
LACAAPPLAGWSRYIPEGLQCSCGIDYYTLKPEVNNESFVIYMFVVHFTIPMIIIFFCY
GQLVFTVKEAAAQQQESATTQKAEKEVTRMVIIMVIAFLICWVPYASVAFYIFTHQG
SNFGPIFMTIPAFFAKSAAIYNPVIYIMMNKQFRNCMLTTICCGKNPLGDDEASATVS
KTETSQVAPA.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleotide substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleotides, or any range derivable therein, of SEQ ID NO:1 or 2.

In some aspects, the dominant mutation of the endogenous RHO gene targeted by gene KI therapy according to the compositions and methods of the disclosure comprises one or more of the following mutations with respect to SEQ ID NO:2 corresponding to the human wild-type RHO gene: P23H, P347L, G51V, and/or T58R. In specific aspects, the dominant mutation of the endogenous RHO gene comprises a P23H with respect to SEQ ID NO:2 corresponding to the human wild-type RHO gene. In specific aspects, the dominant mutation of the endogenous RHO gene comprises a P347L with respect to SEQ ID NO:2 corresponding to the human wild-type RHO gene. In specific aspects, the dominant mutation of the endogenous RHO gene comprises a G51V with respect to SEQ ID NO:2 corresponding to the human wild-type RHO gene. In specific aspects, the dominant mutation of the endogenous RHO gene comprises a T58R with respect to SEQ ID NO:2 corresponding to the human wild-type RHO gene.

B. Autosomal Recessive Disorders

In some aspects, the autosomal disorder to be treated or prevented by the methods and/or compositions disclosed herein is an autosomal recessive disorder. Therefore, in some aspects, the mutated variant of the wild-type gene is a recessive variant, and cells expressing recessive variants are targeted for the purpose of inhibiting or reducing expression of the recessive variants of wild-type genes.

Autosomal recessive is a pattern of inheritance characteristic of some genetic disorders. “Autosomal” means that the gene in question is located on one of the numbered, or non-sex, chromosomes. “Recessive” means that two copies of the mutated gene (one from each parent) are required to cause the disorder. These disorders are usually passed on by two carriers. The health of the carriers is rarely affected, but they have one changed gene (recessive gene) and one unaffected gene (dominant gene) for the condition. Two carriers have a 25% chance of having an unaffected child with two unaffected genes, a 50% chance of having an unaffected child who also is a carrier, and a 25% chance of having an affected child with two recessive changed genes. Autosomal recessive disorders involve autosomes or the non-sex chromosomes and can therefore affect males and females equally.

Examples of autosomal recessive disorders that may be treated or prevented according to the compositions and methods of the present disclosure include, but art not limited to, oculocutaneous albinism, alkaptonuria, Bartter's syndrome, cystic fibrosis, endemic goitrous cretinism, familial amaurotic idiocy, galactosaemia, Gaucher's disease, glycogen storage disease, phenylketonuria, Wilson's disease, sickle cell disease, Tay-Sachs disease, and xeroderma pigmentosa.

C. Mutated Variants of Wild-Type Genes

The nucleotide as well as the protein, polypeptide, and peptide sequences for various genes associated with autosomal disorders that may be selected for or excluded from gene KI therapy according to the compositions and methods of the disclosure have been previously disclosed and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

1. Polypeptides

The protein, polypeptide, and peptide sequences for various genes associated with autosomal disorders that may be selected for or excluded from gene KI therapy according to the compositions and methods of the disclosure may include at least, at most, equal to, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids substitutions, insertions, or deletions, or be at least, at most, equal to, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, at most, equal to, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of a wild-type amino acid sequence (e.g., a wild-type gene coding sequence of a mutated variant of the wild-type gene).

The following is a discussion of changing the amino acid subunits of a protein to create a variant polypeptide or peptide. Such changes may happen spontaneously in vivo, resulting in mutated variants of wild-type genes. For example, certain amino acids may be substituted for other amino acids in a protein or polypeptide sequence with or without appreciable loss of interactive binding capacity with structures such as, for example, binding sites on substrate molecules. Since it is the interactive capacity and nature of a protein that defines that protein's functional activity, certain amino acid substitutions can be made in a protein sequence and in its corresponding DNA coding sequence and produce a protein with similar or different properties. It is thus contemplated by the inventors that various changes may be made in the DNA sequences of genes which encode proteins with or without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect at least, at most, equal to, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least, at most, equal to, or between any two of 50%, 60%, 70%, 80%, or 90%, including all values and ranges there between, identical to any sequence provided or referenced herein. A variant can include at least, at most, equal to, or between any two of 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted, or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine variants of polypeptides as set forth herein using well-known techniques. Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. Any one or more changes disclosed herein may modify an endogenous gene coding sequence to produce a mutated variant of a wild-type gene coding sequence.

2. Nucleic Acids

The protein, polypeptide, and peptide sequences for various genes associated with autosomal disorders that may be selected for or excluded from gene KI therapy according to the compositions and methods of the disclosure may include at least, at most, equal to, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant nucleotide substitutions, insertions, or deletions, or be at least, at most, equal to, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, at most, equal to, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous nucleotides, or any range derivable therein, of a wild-type nucleotide sequence (e.g., a wild-type gene coding sequence of a mutated variant of the wild-type gene).

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides encoding a chimeric polypeptide, polynucleotides encoding a chimeric antigen receptor, polynucleotides encoding an immune cell engager, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the wild-type coding sequence of genes that may be inserted upstream of mutated variants thereof also provided, as well as nucleic acids for transduction or transformation of cells to facilitate insertion of such nucleic acids that encode the wild-type coding sequence of genes that may be inserted upstream of mutated variants thereof. The nucleic acids can be single-stranded or double-stranded and can comprise RNA and/or DNA nucleotides and artificial variants thereof (e.g., peptide nucleic acids).

In certain aspects, there are polynucleotide variants having substantial identity to the wild-type sequences disclosed herein; those comprising at least, at most, equal to, or between any two of 70%, 75%, 80%, 85%, 90%, 95%, 96%, 97%, 98%, 99%, or 1-00% sequence identity, including all values and ranges there between, compared to a polynucleotide sequence provided herein using the methods described herein (e.g., BLAST analysis using standard parameters). In certain aspects, an isolated polynucleotide will comprise a nucleotide sequence encoding a polypeptide that has at least 90%, preferably 95%, or up to 100% identity to a wild-type amino acid sequence described herein, over the entire length of the sequence.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, at least, at most, equal to, or between any two of 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

Changes can be introduced by mutation into a nucleic acid, thereby leading to changes in the amino acid sequence of a polypeptide (e.g., a mutated variant of a wild-type gene) that it encodes. Mutations can be introduced spontaneously in vivo or may be introduced in vitro using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid with or without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. 1 449:581-594 (2013). For example, the mutation can quantitatively or qualitatively change the biological activity. Examples of quantitative changes include increasing, reducing or eliminating the activity. Examples of qualitative changes include altering the binding specificity of a protein.

### III. Vectors

Exogenous nucleic acids encoding the wild-type coding sequence of genes that may be inserted upstream of endogenous mutated variants thereof, as well as nucleic acids for transduction or transformation of cells to facilitate insertion of such exogenous nucleic acids that encode the wild-type coding sequence of genes that may be inserted upstream of mutated variants thereof, may be delivered to recipient cells by any suitable vector, including by a viral vector or by a non-viral vector. The term “vector” includes any genetic element (e.g., a plasmid, a transposon, a cosmid, an artificial chromosome, a viral vector, etc.) that is capable of replicating when associated with the proper control elements. Examples of viral vectors include at least retroviral, lentiviral, adenoviral, or adeno-associated viral vectors. Examples of non-viral vectors include at least plasmids, transposons, lipids, nanoparticles, lipid nanoparticles, and so forth.

In some aspects, the vector(s) is an artificial chromosome. An artificial chromosome is a genetically engineered chromosome that can be used as a vector to carry large DNA inserts. In some aspects, the artificial chromosome is human artificial chromosome (HAC) (see, e.g., Kouprina et al., Expert Opin. Drug Deliv 11(4): 517-535, 2014; Basu et al., Pediatr. Clin. North Am. 53:843-853, 2006; Ren et al., Stem. Cell Rev. 2(1):43-50, 2006; Kazuki et al., Mol. Ther. 19(9):1591-1601, 2011; Kazuki et al., Gen. Ther. 18:384-393, 2011; and Katoh et al., Biochem. Biophys. Res. Commun. 321:280-290, 2004). In some aspects, the vector(s) is a yeast artificial chromosome (YAC) (see, e.g., Murray et al., Nature 305:189-193, 1983; Ikeno et al. (1998) Nat. Biotech. 16:431-439, 1998). In some aspects, the vector(s) is a bacterial artificial chromosome (BAC) (e.g., pBeloBACl 1, pECBAC1, and pBACl08L). In some aspects, the vector(s) is a PI-derived artificial chromosome (PAC). Examples of artificial chromosome are known in the art. In some aspects, the vector(s) is a viral vector (e.g., adeno-associated virus, adenovirus, lentivirus, and retrovirus). Non-limiting examples of viral vectors are described herein. In some aspects, the vector(s) is an adeno-associated viral vector (AAV) (see, e.g., Asokan et al., Mol. Ther. 20:699-7080, 2012).

Recombinant AAV vectors are typically composed of, at a minimum, a transgene or a portion thereof and a regulatory sequence, and optionally 5′ and 3′ AAV inverted terminal repeats (ITRs). Such a recombinant AAV vector is packaged into a capsid and delivered to a selected target cell. The AAV sequences of the vector typically comprise the cis-acting 5′ and 3′ ITR sequences (see, e.g., B. J. Carter, in “Handbook of Parvoviruses,” ed., P. Tijsser, CRC Press, pp. 155 168, 1990). Typical AAV ITR sequences are about 145 nucleotides in length. In some aspects, at least 75% of a typical ITR sequence (e.g., at least 80%, at least 85%, at least 90%, or at least 95%) is incorporated into the AAV vector. The ability to modify these ITR sequences is within the skill of the art (see, e.g., texts such as Sambrook et al., “Molecular Cloning. A Laboratory Manual,” 2d ed., Cold Spring Harbor Laboratory, New York, 1989; and K. Fisher et al., J Virol. 70:520 532, 1996). In some aspects, any of the coding sequences described herein are flanked by 5′ and 3′ AAV ITR sequences in the AAV vectors. The AAV ITR sequences may be obtained from any known AAV, including presently identified AAV types. AAV vectors as described herein may include any of the regulatory elements described herein (e.g., one or more of a promoter, a polyA sequence, and an IRES). In some aspects, the AAV vector is an AAV1 vector, an AAV2 vector, an AAV3 vector, an AAV4 vector, an AAV5 vector, an AAV6 vector, an AAV7 vector, an AAV8 vector, an AAV9 vector, an AAV2.7m8 vector, an AAV8BP2 vector, and an AAV293 vector. Additional exemplary AAV vectors that can be used herein are known in the art. See, e.g., Kanaan et al., Mol. Ther. Nucleic Acids 8:184-197, 2017; Li et al., Mol. Ther. 16(7):1252-1260; Adachi et al., Nat. Commun. 5:3075, 2014; Isgrig et al., Nat. Commun. 10(1):427, 2019; and Gao et al., J. Virol. 78(12):6381-6388.

The vectors provided herein can be of different sizes. The choice of vector that is used in any of the compositions, kits, and methods described herein may depend on the size of the vector.

In some aspects, the vector(s) is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, the vector(s) is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, the vector(s) is a viral vector and can have a total number of nucleotides of up to 10 kb. In some aspects, the viral vector(s) can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, the vector(s) is an adeno-associated virus (AAV vector) and can include a total number of nucleotides of up to 10 kb. In some aspects, the AAV vector(s) can include a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, the vector(s) is an adeno-associated virus (AAV vector) and comprises a nucleotide sequence having at least, at most, exactly, or between any two of 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NOs:31-36.

In some aspects, the exogenous nucleic acids encoding the wild-type coding sequence of genes that may be inserted upstream of endogenous mutated variants thereof, as well as nucleic acids for transduction or transformation of cells to facilitate insertion of such exogenous nucleic acids that encode the wild-type coding sequence of genes that may be inserted upstream of mutated variants thereof, may be delivered to recipient cells by a lipid nanoparticle. In specific aspects, lipids and exogenous nucleic acids can together form nanoparticles, thereby producing exogenous nucleic acid-containing nanoparticles comprising lipids. The lipids can encapsulate or associate with the exogenous nucleic acid in the form of a lipid nanoparticle (LNP) to aid stability, cell entry, and intracellular release of the exogenous nucleic acid/lipid nanoparticles. A LNP can comprise, e.g., a micelle, a solid lipid nanoparticle, a nanoemulsion, a liposome, etc., or a combination thereof. The lipid component of a LNP may include, for example, a cationic lipid, a phospholipid (such as an unsaturated lipid, e.g., DOPE or DSPC), a polymer-lipid conjugate (e.g., a PEGylated lipid), a structural lipid (e.g., cholesterol), an ionizable lipid, a neutral lipid, or any combination thereof. The elements of the lipid component may be provided in specific fractions. Suitable cationic lipids, phospholipids, polymer-lipid conjugates, structural lipids, ionizable lipids, and neutral lipids and the specific fractions at which these lipids should be provided for the compositions and methods of the present disclosure are known in the art. In addition to these lipid components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

The chemical properties of the LNP, LNP suspension, lyophilized LNP composition, or LNP formulation of the present disclosure may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

Provided herein are exemplary vectors that can be used in any of the compositions and methods described herein. A variety of different methods known in the art can be used to introduce any of vectors disclosed herein into a cell. Non-limiting examples of methods for introducing nucleic acid into a mammalian cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes), transduction, microinjection, electroporation, cell squeezing, sonoporation, protoplast fusion, impalefection, hydrodynamic delivery, gene gun, magnetofection, viral transfection, and nucleofection.

In cases wherein the cell is transduced with a vector encoding exogenous nucleic acids encoding the wild-type coding sequence of genes that may be inserted upstream of endogenous mutated variants thereof, and also requires transduction of another gene or genes into the cell, such as a gene editing technology and/or a selectable marker, the nucleic acid and the gene editing technology may or may not be comprised on or with the same vector. In some cases, the nucleic acid, the gene editing technology (or an element thereof, e.g., a Cas protein or a guide RNA molecule), and/or the selectable marker are expressed from the same vector molecule, such as the same viral vector molecule. In such cases, the expression of the nucleic acid, the gene editing technology (or an element thereof, e.g., a Cas protein or a guide RNA molecule), and/or the selectable marker may or may not be regulated by the same regulatory element(s). When the nucleic acid, the gene editing technology (or an element thereof, e.g., a Cas protein or a guide RNA molecule), and/or the selectable marker are on the same vector, they may or may not be expressed as separate polypeptides. In cases wherein they are expressed as separate polypeptides, they may be separated on the vector by a 2A element or IRES element (or both kinds may be used on the same vector once or more than once), for example.

A. General Aspects

One of skill in the art would be well-equipped to construct a vector through standard recombinant techniques (see, e.g., Sambrook et al., 2001, and Ausubel et al., 1996, both incorporated herein by reference in their entirety) for the expression of the viral proteins and/or antigen receptors of the present disclosure. Vectors can include control sequences including a transcription initiation sequence, a transcription termination sequence, a promoter sequence, an enhancer sequence, an RNA splicing sequence, a polyadenylation (polyA) sequence, a Kozak consensus sequence, untranslated regions, or selectable or screenable markers. Any one or more of the foregoing sequences may be included in a vector, according to some aspects of the disclosure. Any one or more of the foregoing sequences may be excluded from a vector, in some aspects.

1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example.

2. Promoter/Enhancers

The expression cassettes provided herein comprise a promoter to drive expression of the viral protein and/or antigen receptor and other cistron gene products. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, e.g., Sambrook et al. 1989, incorporated herein by reference in its entirety). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7, or SP6 cytoplasmic expression system is another possible aspect. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, RNA polymerase III promoters (e.g., U6), SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain aspects, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure. In specific aspects, the promoter is U6.

In specific aspects, the promoter is a tissue-specific promoter. Exemplary tissue-specific promoters include but are not limited to the following: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, an alpha-myosin heavy chain (a-MHC) promoter, and a cardiac Troponin T (cTnT) promoter. Additional exemplary promoters include Beta-actin promoter, hepatitis B virus core promoter, alpha-fetoprotein (AFP) promoter, bone osteocalcin promoter; bone sialoprotein promoter, CD2 promoter; immunoglobulin heavy chain promoter; T cell receptor alpha-chain promoter, neuronal such as neuron-specific enolase (NSE) promoter, neurofilament light-chain gene promoter, and the neuron-specific vgf gene promoter. In specific aspects, the tissue-specific promoter is a retina-specific promoter. In specific aspects, the tissue specific promoter is human rhodopsin kinase 1 promoter (hRK), which has a length of 292 base pairs and is active and specific for rod and cone photoreceptors. Other tissue-specific promoters contemplated for use in vectors disclosed herein include VE-cadherin/Cadherin 5 (CDH5)/CD144 promoter, Human vitelliform macular dystrophy/Bestrophin 1 promoter, hIRBP enhancer fused to cone transducin alpha promoter, and Human red opsin promoter, however any other promoter that is useful to drive expression of the therapeutic gene is the retina applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

3. Poly(A) Sequences

In some aspects, any of the vectors provided herein can include a poly(A) sequence. Most nascent eukaryotic mRNAs possess a poly(A) tail at their 3′ end which is added during a complex process that includes cleavage of the primary transcript and a coupled polyadenylation reaction (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some aspects, the poly(A) sequence is positioned 3′ to the exogenous nucleic acid sequence encoding the wild-type gene.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, the poly(A) tail is added onto transcripts that contain a specific sequence, the polyadenylation signal or “poly(A) sequence.” The poly(A) tail and the protein bound to it aid in protecting mRNA from degradation by exonucleases.

Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A) sequence” is a sequence that triggers the endonuclease cleavage of an mRNA and the additional of a series of adenosines to the 3′ end of the cleaved mRNA. There are several poly(A) sequences that can be used, including those derived from bovine growth hormone (bgh), mouse-P-globin, mouse-a-globin, human collagen, polyoma virus, the Herpes simplex virus thymidine kinase gene (HSV TK), IgG heavy-chain gene polyadenylation signal), human growth hormone (hGH), or SV40 poly(A) site, such as the SV40 late and early poly(A) site. The poly(A) sequence can a sequence of AATAAA. The AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG.

4. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. The Kozak consensus sequence (“Kozak consensus” or “Kozak sequence”) is a nucleic acid motif that functions as the protein translation initiation element in most eukaryotic mRNA transcripts. The sequence is defined as 5′-gccgccRccAUGG-3′ (SEQ ID NO:3), where the underlined nucleotides indicate the translation start codon, coding for Methionine; upper-case letters indicate highly conserved bases, e.g., the “AUGG” sequence; “R” indicates that a purine (adenine or guanine) is observed at this position; and a lower-case letter denotes the most common base at a position where the base can nevertheless vary. One of ordinary skill in the art would readily be capable of determining the appropriate sequence for protein translation initiation elements and providing the necessary signals. It is well known that initiation codon for endogenous gene coding sequences must be “in-frame” with the reading frame of the desired coding sequence to ensure translation of the entire insert. Exogenous translational control signals and initiation codons can be either natural or synthetic, and in some aspects, exogenous translational control elements may be out-of-frame with the reading frame of the endogenous coding sequence. The efficiency of expression may be enhanced by the inclusion of appropriate transcription enhancer elements.

In certain aspects, the use of internal ribosome entry sites (IRES) elements is used to create multigene, or polycistronic messages. IRES elements are able to bypass the ribosome scanning model of 5′ methylated Cap dependent translation and begin translation at internal sites. There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific aspects, in a single vector the multiple 2A sequences are non-identical, although in alternative aspects the same vector utilizes two or more of the same 2A sequences. Examples of 2A sequences are provided in US 2011/0065779 which is incorporated by reference herein in its entirety.

5. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

6. Untranslated Regions (UTRs)

In some aspects, any of the vectors described can include an untranslated region. In some aspects, a vector can include a 5′ UTR or a 3′ UTR. Untranslated regions (UTRs) of a gene are transcribed but not translated. The 5′ UTR starts at the transcription start site and continues to the transcription initiation element and start codon but does not include the start codon. The 3′ UTR starts immediately following the stop codon and continues until the transcriptional termination signal. The regulatory features of a UTR can be incorporated into any of the vectors, compositions, kits, or methods as described herein.

Natural 5′ UTRs include a sequence that plays a role in translation initiation. They harbor signatures like Kozak sequences, which are commonly known to be involved in the process by which the ribosome initiates translation of many genes. For example, in some aspects, a 5′ UTR is included in any of the vectors described herein. Non-limiting examples of 5′ UTRs including those from the following genes: albumin, serum amyloid A, Apolipoprotein AB/E, transferrin, alpha fetoprotein, erythropoietin, and Factor VIII, can be used to enhance expression of a nucleic acid molecule, such as a mRNA. In some aspects, a 5′ UTR from a mRNA that is transcribed by a cell can be included in any of the vectors, compositions, kits, and methods described herein.

3′ UTRs are known to have stretches of adenosines and uridines embedded in them. These AU-rich signatures are particularly prevalent in genes with high rates of turnover. Most proteins binding to the AU-rich elements are known to destabilize the messenger, whereas members of the ELAV family, most notably HuR, have been documented to increase the stability of mRNA. HuR binds to AREs of all the three classes.

In some aspects of any of the compositions described herein, a 5′ UTR, a 3′ UTR, or both are included in a vector (e.g., any of the vectors described herein). For example, any of the 5′ UTRs described herein can be operatively linked to the start codon in any of the coding sequences described herein. For example, any of the 3′ UTRs can be operatively linked to the 3′-terminal codon (last codon) in any of the coding sequences described herein.

In other aspects, non-UTR sequences may be incorporated into the 5′ or 3′ UTRs. In some aspects, introns or portions of intron sequences may be incorporated into the flanking regions of the polynucleotides in any of the vectors, compositions, kits, and methods provided herein. Incorporation of intronic sequences may increase protein production as well as mRNA levels.

7. Selection and Screenable Markers

In some aspects, cells comprising exogenous nucleic acids encoding the wild-type coding sequence of genes that may be inserted upstream of endogenous mutated variants thereof, or nucleic acids for transduction or transformation of cells to facilitate insertion of such exogenous nucleic acids that encode the wild-type coding sequence of genes that may be inserted upstream of endogenous mutated variants thereof, of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP and mCherry, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Any of the vectors provided herein can optionally include a sequence encoding a reporter protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with regulatory elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (MA), and immunohistochemistry).

B. Multicistronic Vectors

In particular aspects, the exogenous nucleic acids encoding the wild-type coding sequence of genes that may be inserted upstream of endogenous mutated variants thereof, or nucleic acids for transduction or transformation of cells to facilitate insertion of such exogenous nucleic acids that encode the wild-type coding sequence of genes that may be inserted upstream of endogenous mutated variants thereof, are expressed from a multicistronic vector (the term “cistron” as used herein refers to a nucleic acid sequence from which a gene product may be produced). In specific aspects, the multicistronic vector encodes the nucleic acid, a gene editing technology (or an element thereof), and/or a selectable marker. In some cases, the multicistronic vector encodes at least one exogenous nucleic acid encoding the wild-type coding sequence of a gene that may be inserted upstream of an endogenous mutated variant thereof and one or more elements of a gene editing technology (e.g., a Cas protein or a guide RNA molecule). In some cases, the multicistronic vector encodes at least one exogenous nucleic acid encoding the wild-type coding sequence of a gene that may be inserted upstream of an endogenous mutated variant thereof, one or more elements of a gene editing technology (e.g., a Cas protein or a guide RNA molecule), and a selectable marker.

In certain aspects, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific aspects, one or more of the genes expressed by the vector includes one, two, or more exogenous wild-type coding sequences of a gene that may be inserted upstream of endogenous mutated variant thereof and one or more elements of a gene editing technology (e.g., a Cas protein and/or a guide RNA molecule). The vector may further comprise one or more selectable markers or reporters, for example fluorescent or enzymatic reporters (e.g., GFP, mCherry), such as for cellular assays and animal imaging.

In specific cases, the vector may comprise at least 1, 2, 3, 4, or more cistrons separated by cleavage sites of any kind, such as 2A cleavage sites. The vector may or may not be adenovirus-associated virus (AAV)-based including 3′ and 5′ ITRs with pAAV rep/Cap 2/2, 2/8, 2/7m8. The vector may comprise 1, 2, 3, 4, or more cistrons with one, two, three, or more 2A cleavage sites and multiple ORFs for gene swapping. The system allows for combinatorial overexpression of multiple genes that are flanked by restriction site(s) for rapid integration through subcloning, and the system also includes at least 2A self-cleavage sites, in some aspects. Thus, the system allows for expression of multiple nucleic acids encoding the exogenous wild-type coding sequences of genes that may be inserted upstream of endogenous mutated variants thereof, one or more elements of a gene editing technology (e.g., a Cas protein or a guide RNA molecule), and selectable markers. This system may also be applied to other viral and non-viral vectors, including but not limited lentivirus, as well as non-viral plasmids.

Aspects of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has aspects wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Aspects of the disclosure include polycistronic vectors comprising at least one, at least two, at least three, or at least four cistrons each flanked by one or more restriction enzyme sites, wherein at least one cistron encodes for at least one exogenous wild-type coding sequence of a gene, one or more elements of a gene editing technology (e.g., a Cas protein or a guide RNA molecule), and a selectable marker. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self-cleavage site, such as a 2A self-cleavage site. In some cases each of the cistrons express separate polypeptides from the vector. On particular cases, adjacent cistrons on the vector are separated by an IRES element.

In certain aspects, the present disclosure provides a system for cell engineering allowing for combinatorial expression, including overexpression, of multiple cistrons that may include one, two, or more exogenous wild-type coding sequences of genes, one or more elements of a gene editing technology (e.g., a Cas protein or a guide RNA molecule), and selectable markers, for example. In particular aspects, the use of a polycistronic vector as described herein allows for the vector to produce equimolar levels of multiple gene products from the same mRNA. The multiple genes may comprise, but are not limited to, wild-type coding sequences of genes, one or more elements of a gene editing technology (e.g., a Cas protein or a guide RNA molecule), selectable markers, and so forth.

In specific aspects, the vector is a viral vector (retroviral vector, lentiviral vector, adenoviral vector, or adeno-associated viral vector, for example) or a non-viral vector. When 2A cleavages sites are utilized in the vector, the 2A cleavage site may comprise a P2A, T2A, E2A and/or F2A site. A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

In aspects wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (Thosea asigna virus 2A) were also identified. The mechanism of 2A-mediated “self-cleavage” was discovered to be ribosome skipping the formation of a glycyl-prolyl peptide bond at the C-terminus of the 2A.

### IV. Gene Editing of Cells

In particular aspects, cells comprising exogenous nucleic acids encoding the wild-type coding sequence of genes that may be inserted upstream of mutated variants thereof are gene edited to modify expression of one or more endogenous genes in the cell. In specific cases, cells are modified to have reduced levels of expression of one or more endogenous genes, including inhibition of expression of one or more endogenous genes (that may be referred to as knocked out). Such cells may or may not be expanded.

In some aspects, the nucleic acids are introduced alone or as part of engineered constructs via stable viral vectors, in other aspects the polynucleotides can be introduced by electroporation for transient expression of mRNA that would be translated to protein inside the cells, and in other aspects the polynucleotides can be introduced using knock-in approaches using gene editing technologies including but not limited to CRISPR, TALENs, Zinc fingers, and/or retrons, among others. The knock-in approaches can introduce the polynucleotides in specific favorable genomic locations and/or under the appropriate conditions to direct high-level expression or to inhibit expression of endogenous genes. Knock-in approaches may exploit CRISPR/Cas9 mediated homology-independent targeted integration, which is described in, e.g., Suzuki K. et al. (2016). Nature 540:144-149, incorporated by reference herein in its entirety.

In particular cases, one or more endogenous genes of the cells are modified, such as disrupted in expression where the expression is reduced in part or in full. In specific cases, one or more genes are knocked down or knocked out using processes of the disclosure. In specific cases, multiple genes are knocked down or knocked out, and this may or may not occur in the same step in their production. In specific cases, one or more genes are knocked in using processes of the disclosure; knock-in of one or more genes may result in knock-down or knock-out of one or more endogenous genes. In specific cases, multiple genes are knocked in, and this may or may not occur in the same step in their production.

The genes that are edited in or out of cells may be of any kind, but in specific aspects the genes are genes whose gene products associated with autosomal dominant disorders, such as RHO, as one example.

A. DNA-Binding Nucleic Acids

In some aspects, the gene editing is carried out using one or more DNA-binding nucleic acids, such as alteration via an RNA-guided endonuclease (RGEN) or retron library recombineering.

1. CRISPR/Cas

In some aspects, gene editing can be carried out using clustered regularly interspaced short palindromic repeats (CRISPR) and CRISPR-associated (Cas) proteins; in some aspects, CpF1 is utilized instead of Cas9. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other aspects, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other aspects, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some aspects, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (Cas12a) homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases, Cpf1 (Cas12a) may be used as an endonuclease instead of Cas9. The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some aspects, a Cas9 nickase may be used in combination with guide sequence(s), e.g., two guide sequences, which target respectively sense and antisense strands of the DNA target. This combination allows both strands to be nicked and used to induce non-homologous end joining (NHEJ) or homology-directed repair (HDR) or homology-independent targeted integration (HITI).

In some aspects, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some aspects, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

1. Retrons

In some aspects, the gene editing is carried out using retrons and retron recombineering. A retron is a distinct DNA sequence found in the genome of many bacteria species that codes for reverse transcriptase and a unique single-stranded DNA/RNA hybrid called multicopy single-stranded DNA (msDNA). Retron msr RNA is the non-coding RNA produced by retron elements and is the immediate precursor to the synthesis of msDNA. Retron elements are about 2000 kb long. They contain a single operon controlling the synthesis of an RNA transcript carrying three loci, msr, msd, and ret, that are involved in msDNA synthesis. The DNA portion of msDNA is encoded by the msd gene, the RNA portion is encoded by the msr gene, while the product of the ret gene is a reverse transcriptase similar to the RTs produced by retroviruses and other types of retroelements. Like other reverse transcriptases, the retron RT contains seven regions of conserved amino acids, including a highly conserved tyr-ala-asp-asp (YADD) sequence associated with the catalytic core. The ret gene product is responsible for processing the msd/msr portion of the RNA transcript into msDNA.

The retron msr RNA folds into a characteristic secondary structure that contains a conserved guanosine residue at the end of a stem loop. Synthesis of DNA by the retron-encoded reverse transcriptase (RT) results in a DNA/RNA chimera which is composed of small single-stranded DNA linked to small single-stranded RNA. The RNA strand is joined to the 5′ end of the DNA chain via a 2′-5′ phosphodiester linkage that occurs from the 2′ position of the conserved internal guanosine residue.

Materials and methods for gene editing using retrons and retron recombineering are disclosed in, e.g., Schubert M. G. et al. (April 2021). PNAS 118 (18):e2018181118, incorporated by reference herein in its entirety.

B. Nucleases

In some aspects, the gene editing is carried out using one or more nucleases, such as one or more transcription activator-like effector nucleases (TALENs) and/or zinc-finger nucleases (ZFNs).

1. TALENs

TALENs are DNA-binding restriction enzymes engineered to cut specific sequences of DNA and can be made by fusing a transcription activator-like (TAL) effector DNA-binding domain to a DNA cleavage domain (a nuclease). TAL effectors are proteins secreted by Xanthomonas bacteria. The DNA binding domain contains a repeated highly conserved sequence of about 33-34 amino acids with divergent 12th and 13th amino acids, referred to as the Repeat Variable Diresidue (RVD), that are highly variable and show a strong correlation with specific nucleotide recognition. In some aspects, specific DNA-binding domains are engineered by selecting a combination of repeat segments containing the appropriate RVDs, and slight changes in the RVD and the incorporation of “nonconventional” RVD sequences can improve targeting specificity. The non-specific DNA cleavage domain from the end of the FokI endonuclease and/or variants thereof can be used to construct hybrid nucleases. The FokI domain functions as a dimer, having two constructs with unique DNA binding domains for sites in the target genome with proper orientation and spacing. Both the number of amino acid residues between the TAL effector DNA binding domain and the FokI cleavage domain and the number of bases between the two individual TALEN binding sites can be varied, in some aspects, to achieve high levels of activity and/or specificity.

TALEN constructs may be generated using publicly available software programs (e.g., DNAWorks) to calculate oligonucleotides suitable for assembly in a two-step PCR oligonucleotide assembly followed by whole gene amplification. Additionally, or alternatively, a number of modular assembly schemes may be used, such as those described in Cermak T. et al. (July 2011). Nucleic Acids Research. 39 (12): e82; Zhang F. (February 2011) et al. Nature Biotechnology. 29 (2): 149-53; Morbitzer R. et al. (July 2011). Nucleic Acids Research. 39 (13): 5790-9; Li T. et al. (August 2011). Nucleic Acids Research. 39 (14): 6315-25; Geissler R. et al. (2011). PLOS ONE. 6 (5): e19509; and Weber E. et al. (2011). PLOS ONE. 6 (5): e19722, all of which are incorporated by reference herein in their entirety. Once TALEN constructs have been assembled, they may be inserted into a viral or non-viral vector; target cells are then transfected with the vector, and gene products are expressed and can enter the nucleus to access the genome. TALEN can be used to edit genomes by inducing double-strand breaks (DSB), which cells respond to with repair mechanisms (e.g., non-homologous end joining and/or homology directed repair). Additionally, or alternatively, TALEN constructs can be delivered to the cells as mRNAs.

2. ZFNs

ZFNs are restriction enzymes generated by fusing a zinc finger DNA-binding domain to a DNA-cleavage domain. In some aspects, zinc finger domains can be engineered to target specific desired DNA sequences to enable zinc-finger nucleases to target unique sequences in genomes. The DNA-binding domains can contain between three and six individual zinc finger repeats (e.g., 3, 4, 5, or 6 repeats) and can each recognize between 9 and 18 base pairs (e.g., 9, 10, 11, 12, 13, 14, 15, 16, 17, or 18 base pairs). Upon recognition of a 3 base pair DNA sequence, ZFNs can generate a 3-finger array that can recognize a 9 base pair target site. Additionally, or alternatively, ZFNs can utilize either 1-finger or 2-finger modules to generate zinc-finger arrays with six or more individual zinc fingers. ZFN DNA-binding domains may be selected using, e.g., phage display, yeast one-hybrid systems, bacterial one-hybrid and two-hybrid systems, and mammalian cells to select proteins that bind a given DNA target from a large pool of partially randomized zinc-finger arrays. In some aspects, a bacterial two-hybrid system is used and combines pre-selected pools of ZFNs selected to bind a given 3 base pair DNA sequence followed by a second round of selection to obtain 3-finger arrays capable of binding a desired 9 base pair sequence. See, e.g., Maeder M L, et al. (September 2008). Mol. Cell. 31 (2): 294-301, incorporated by reference herein in its entirety.

The non-specific DNA cleavage domain (e.g., from the type IIs restriction endonuclease FokI) can be used as the cleavage domain in ZFNs. This cleavage domain dimerizes to cleave DNA, and in some aspects, a pair of ZFNs is used to target non-palindromic DNA sites. Standard ZFNs fuse the cleavage domain to the C-terminus of each zinc finger domain. To let the two cleavage domains dimerize and cleave DNA, the two individual ZFNs bind opposite strands of DNA with their C-termini a certain distance apart. In some aspects, the 5′ edge of each binding site is separated by 5 to 7 base pairs for the linker sequences between the zinc finger domain and the cleavage domain. Several different protein engineering techniques have been employed to improve both the activity and specificity of the nuclease domain used in ZFNs. For example, in some aspects, a FokI variant with enhanced cleavage activity generated using directed evolution is employed. See, e.g., Guo J. et al. (2010). Journal of Molecular Biology. 400 (1): 96-107, incorporated by reference herein in its entirety. Additional or alternatively, structure-based design can be employed to improve the cleavage specificity of FokI by modifying the dimerization interface so that only the intended heterodimeric species are active.

In some aspects, zinc-finger nickases (ZFNickases) may be used. ZFNickases can be created by inactivating the catalytic activity of one ZFN monomer in the ZFN dimer required for double-strand cleavage. ZFNickases demonstrate strand-specific nicking activity in vitro and can provide for highly specific single-strand breaks in DNA, which undergo the same cellular mechanisms for DNA that ZFNs exploit but show a significantly reduced frequency of mutagenic NHEJ repairs at their target nicking site. This reduction can bias for homologous recombination (HR)-mediated gene modifications.

### V. General Methods of Treatment

In various aspects, diseased or other cells expressing endogenous mutated variants of wild-type genes (i.e., mutated gene variants) are targeted for the purpose of improving a medical condition in an individual that has the medical condition or for the purpose of reducing the risk or delaying the severity and/or onset of the medical condition in an individual. In specific cases, cells expressing endogenous mutated variants of wild-type genes (i.e., mutated gene variants) are targeted for the purpose of inhibiting or reducing expression of the endogenous mutated variants of wild-type genes.

Nucleic acids for transduction or transformation of cells to facilitate insertion of exogenous nucleic acids that encode the wild-type coding sequence of genes that may be inserted upstream of endogenous mutated variants thereof as contemplated herein, and/or pharmaceutical compositions comprising the same, are used for the prevention, treatment or amelioration of a disease, such as an autosomal disease (i.e., an autosomal dominant disorder or an autosomal recessive disorder).

The cells for which the exogenous nucleic acids that encode the wild-type coding sequence of genes that may be inserted upstream of endogenous mutated variants thereof are utilized may be any cells expressing mutated variants of wild-type genes, and cells engineered for cell therapy for subjects, in particular aspects. In specific aspects, the cells have been engineered to express one or more wild-type genes. In some aspects, cells of the disclosure have been engineered to express one or more wild-type genes and have reduced expression of or do not express an endogenous mutated variant of the wild-type gene.

In particular aspects, the present disclosure contemplates, in part, exogenous nucleic acids that encode the wild-type coding sequence of genes that may be inserted upstream of endogenous mutated variants thereof that can be administered either alone or in any combination using standard vectors and/or gene delivery systems, and in at least some aspects, together with a pharmaceutically acceptable carrier or excipient. In certain aspects, subsequent to administration, the nucleic acid molecules or vectors may be stably integrated into the genome of the subject. In specific aspects, viral vectors may be used that are specific for certain cells or tissues and persist in cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of autosomal diseases (i.e., autosomal dominant disorders or autosomal recessive disorders) comprising the step of administering to a subject in the need thereof an effective amount of exogenous nucleic acids encoding the wild-type coding sequence of genes that may be inserted upstream of endogenous mutated variants thereof, and nucleic acids for transduction or transformation of cells to facilitate insertion of such exogenous nucleic acids that encode the wild-type coding sequence of genes that may be inserted upstream of endogenous mutated variants thereof, as contemplated herein and/or produced by a process as contemplated herein.

Possible indications for administration of the composition(s) are autosomal dominant disorders, including achondroplasia, acute intermittent porphyria, antithrombin III deficiency, BRCA1/BRCA2 positive breast cancer, cherubism, dominant blindness (e.g., Leber congenital amaurosis, retinitis pigmentosa, Stargardt-like macular dystrophy, stationary night blindness, vitreoretinochoroidopathy), dominant congenital deafness, Ehlers-Danlos syndrome, familial adenomatous polyposis, Gilbert's disease, hereditary hemorrhagic telangiectasia, hereditary elliptosis, hereditary spherocytosis, holoproencephaly, Huntington's disease, hypercholesterolemia, idiopathic hypoparathyroidism, intestinal polyposis, marble bone disease, Marfan's syndrome, myotonic dystrophy, neurofibromatosis, osteogenesis imperfecta, polycystic kidney disease, protein C deficiency, retinitis pigmentosa, retinoblastoma, Treacher Collins syndrome, tuberous sclerosis, or Von Willebrand's disease, for example. The administration of the composition(s) of the disclosure is useful for all stages and types of autosomal dominant disorders.

Possible indications for administration of the composition(s) are autosomal recessive disorders, including oculocutaneous albinism, alkaptonuria, Bartter's syndrome, cystic fibrosis, endemic goitrous cretinism, familial amaurotic idiocy, galactosaemia, Gaucher's disease, glycogen storage disease, phenylketonuria, Wilson's disease, sickle cell disease, Tay-Sachs disease, and xeroderma pigmentosa. The administration of the composition(s) of the disclosure is useful for all stages and types of autosomal recessive disorders.

Accordingly, provided herein in some aspects are methods of introducing into a subject a therapeutically effective amount of any of the compositions described herein. Also provided are methods of increasing expression of a wild-type gene (e.g., a wild-type gene from which a mutated gene variant is derived) in cells that include introducing into the cells of the subject a therapeutically effective amount of any of the compositions described herein. Also provided are methods of inhibiting or decreasing expression of a mutated variant of a wild-type gene in cells (e.g., a wild-type gene from which a mutated gene variant is derived) that include introducing into the cells of the subject a therapeutically effective amount of any of the compositions described herein. Also provided are methods of treating an autosomal disorder (i.e., an autosomal dominant disorder or an autosomal recessive disorder) in a subject identified as expressing a mutated gene variant, where the methods include administering a therapeutically effective amount of any of the compositions described herein into cells of a subject.

In some aspects of any of these methods, the mammal has been previously identified as having a defective wild-type gene (e.g., a gene having a mutation that results in abnormal expression and/or activity of a protein encoded by the gene). Some aspects of any of these methods further include, prior to the introducing or administering step, determining that the subject has a defective wild-type gene (e.g., a gene having a mutation that results in abnormal expression and/or activity of a protein encoded by the gene). Some aspects of any of these methods can further include detecting a mutation in a wild-type gene in a subject. Some aspects of any of the methods can further include identifying or diagnosing a subject as having an autosomal disorder (i.e., an autosomal dominant disorder or an autosomal recessive disorder).

In some aspects of any of these methods, two or more doses of any of the compositions described herein are introduced or administered into the or subject. Some aspects of any of these methods can include introducing or administering a first dose of the composition into the subject, assessing a phenotype of the subject following the introducing or the administering of the first dose, and administering an additional dose of the composition into the subject found not to have a normal phenotype (e.g., as determined using any test for hearing known in the art).

In some aspects of any of the methods described herein, the composition can be formulated for parenteral administration. In some aspects of any of the methods described herein, the compositions described herein can be administered via local or systemic injection. In some aspects of any of the methods described herein, the compositions are administered through the use of a medical device.

In some aspects of any of the methods described herein, the subject or mammal has or is at risk of developing an autosomal dominant disorder, including achondroplasia, acute intermittent porphyria, antithrombin III deficiency, BRCA1/BRCA2 positive breast cancer, cherubism, dominant blindness (e.g., Leber congenital amaurosis, retinitis pigmentosa, Stargardt-like macular dystrophy, stationary night blindness, vitreoretinochoroidopathy), dominant congenital deafness, Ehlers-Danlos syndrome, familial adenomatous polyposis, Gilbert's disease, hereditary hemorrhagic telangiectasia, hereditary elliptosis, hereditary spherocytosis, holoproencephaly, Huntington's disease, hypercholesterolemia, idiopathic hypoparathyroidism, intestinal polyposis, marble bone disease, Marfan's syndrome, myotonic dystrophy, neurofibromatosis, osteogenesis imperfecta, polycystic kidney disease, protein C deficiency, retinitis pigmentosa, retinoblastoma, Treacher Collins syndrome, tuberous sclerosis, or Von Willebrand's disease, for example. In some aspects of any of the methods described herein, the subject or mammal has been previously identified as having a mutation in a wild-type gene (e.g., a wild-type gene from which a mutated gene variant is derived). In some aspects of any of the methods described herein, the subject or mammal has any of the mutations in a wild-type gene (e.g., a wild-type gene from which a mutated gene variant is derived) that are described herein or are known in the art to be associated with an autosomal dominant disorder.

some aspects of any of the methods described herein, the subject or mammal has or is at risk of developing an autosomal recessive disorder, including oculocutaneous albinism, alkaptonuria, Bartter's syndrome, cystic fibrosis, endemic goitrous cretinism, familial amaurotic idiocy, galactosaemia, Gaucher's disease, glycogen storage disease, phenylketonuria, Wilson's disease, sickle cell disease, Tay-Sachs disease, and xeroderma pigmentosa. In some aspects of any of the methods described herein, the subject or mammal has been previously identified as having a mutation in a wild-type gene (e.g., a wild-type gene from which a mutated gene variant is derived). In some aspects of any of the methods described herein, the subject or mammal has any of the mutations in a wild-type gene (e.g., a wild-type gene from which a mutated gene variant is derived) that are described herein or are known in the art to be associated with an autosomal recessive disorder.

In some aspects of any of the methods described herein, the subject has been identified as having a mutation in a wild-type gene (e.g., a wild-type gene from which a mutated gene variant is derived) and has been diagnosed with an autosomal disorder (i.e., an autosomal dominant disorder or an autosomal recessive disorder). In some aspects of any of the methods described herein, the subject has been identified as having an autosomal disorder (i.e., an autosomal dominant disorder or an autosomal recessive disorder).

Also provided herein are methods of increasing expression of a wild-type gene (e.g., a wild-type gene from which a mutated gene variant is derived) in a cell that include introducing any of the compositions described herein into the mammalian cell. In some aspects of these methods, the cell is in vivo. In some aspects of these methods, the mammalian cell is in a mammal. In some aspects of these methods, the cell is originally obtained from a subject and is cultured ex vivo. In some aspects, the cell has previously been determined to have a defective wild-type gene (e.g., a wild-type gene from which a mutated gene variant is derived). Methods for introducing any of the compositions described herein into a cell are known in the art (e.g., via lipofection or through the use of a viral vector, e.g., any of the viral vectors described herein).

The disclosure further encompasses co-administration protocols with other compounds. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

### VI. General Pharmaceutical Compositions

In some aspects, pharmaceutical compositions are administered to a subject. Different aspects may involve administering an effective amount of a composition to a subject. In some aspects, nucleic acids encoding the wild-type coding sequence of genes that may be inserted upstream of mutated variants thereof, as well as nucleic acids for transduction or transformation of cells to facilitate insertion of such nucleic acids that encode the wild-type coding sequence of genes that may be inserted upstream of mutated variants thereof, may be delivered to the subject to protect against or treat a condition (e.g., an autosomal disorder). Alternatively, an expression vector encoding nucleic acids encoding the wild-type coding sequence of genes that may be inserted upstream of mutated variants thereof, as well as nucleic acids for transduction or transformation of cells to facilitate insertion of such nucleic acids that encode the wild-type coding sequence of genes that may be inserted upstream of mutated variants thereof, may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intrapleural, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to, intraorbital or intraretinal administration. In some aspects, the composition is administered intravenously, intramuscularly, intrapleurally, subcutaneously, topically, orally, transdermally, intraperitoneally, intraorbitally, by implantation, by inhalation, intrathecally, intraventricularly, or intranasally.

Upon formulation, solutions will be administered in a manner compatible with the dosage formulation and in such amount as is therapeutically or prophylactically effective. The formulations are easily administered in a variety of dosage forms, such as the type of injectable solutions described above.

The appropriate dosage may be determined based on the type of disease to be treated, severity and course of the disease, the clinical condition of the individual, the individual's clinical history and response to the treatment, and the discretion of the attending physician. Such compositions would normally be administered as pharmaceutically acceptable compositions that include physiologically acceptable carriers, buffers or other excipients.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects in which the composition comprises a viral vector, between 1×10⁶ to about 1×10¹⁶ vg/ml are included in the composition. In some aspects, 1×10¹⁵ vg/ml are included in the composition. In some aspects, greater than 1×10⁶ vg/ml are included. In some aspects, greater than 1×10⁷ vg/ml are included. In some aspects, greater than 1×10⁸ vg/ml are included. In some aspects, greater than 1×10⁹ vg/ml are included. In some aspects, greater than 1×10¹⁰ vg/ml are included. In some aspects, greater than 1×10¹¹ vg/ml are included. In some aspects, greater than 1×10¹² vg/ml are included. In some aspects, greater than 1×10¹³ vg/ml are included. In some aspects, greater than 1×10¹⁴ vg/ml are included. In some aspects, greater than 1×10¹⁵ vg/ml are included.

In certain aspects, the effective dose of the pharmaceutical composition is one which can provide a blood level of about 1 μM to 150 04. In another aspect, the effective dose provides a blood level of about 4 μM to 100 04.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6, 7, 8, 9, 10, or more administrations. The administrations can be at intervals of at least, at most, equal to, or between any two of, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, weeks, months, or years, including all ranges there between.

### VII. Kits

Aspects relate to a kit comprising a construct as defined herein, a nucleic acid sequence as defined herein, a vector as defined herein and/or a host cell (such as an immune cell) as defined herein. It is also contemplated that the kit of this disclosure comprises a pharmaceutical composition as described herein above, either alone or in combination with further medicaments to be administered to an individual in need of medical treatment or intervention.

In a non-limiting example, cells, reagents to produce cells, vectors, and reagents to produce vectors and/or components thereof may be comprised in a kit. In certain aspects, cells may be comprised in a kit, and they may or may not express a nucleic acid of the disclosure, a gene editing technology (or an element thereof, e.g., a Cas protein or a guide RNA molecule), and/or the selectable marker. Such a kit may or may not have one or more reagents for manipulation of cells. Such reagents include small molecules, proteins, nucleic acids, antibodies, buffers, primers, nucleotides, salts, and/or a combination thereof, for example. Nucleic acids that encode one or more wild-type coding sequences of genes that may be inserted upstream of mutated variants thereof, one or more gene editing technologies (or an element thereof, e.g., a Cas protein or a guide RNA molecule), and/or one or more selectable marker may be included in the kit, including reagents to generate same. Proteins, such as cytokines or antibodies, including monoclonal antibodies, may be included in the kit.

In particular aspects, the kit comprises a gene therapy of the disclosure and also another therapy. In some cases, the kit, in addition to the cell therapy aspects, also includes a second therapy, such as chemotherapy, hormone therapy, and/or immunotherapy, for example. The kit(s) may be tailored to a particular disorder (e.g., a particular autosomal disorder) for an individual and comprise respective second therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also may generally contain a second, third or other additional container into which the additional components may be separately placed. However, various combinations of components may be comprised in a vial. The kits of the present disclosure also will typically include a means for containing the composition and any other reagent containers in close confinement for commercial sale. Such containers may include injection or blow-molded plastic containers into which the desired vials are retained.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Mutation-Independent Gene Knock-In Therapy Targeting 5′ UTR for Autosomal Dominant Retinitis Pigmentosa

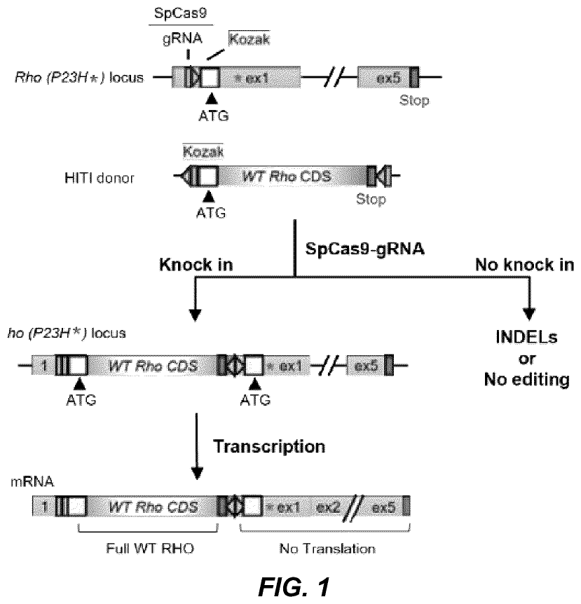

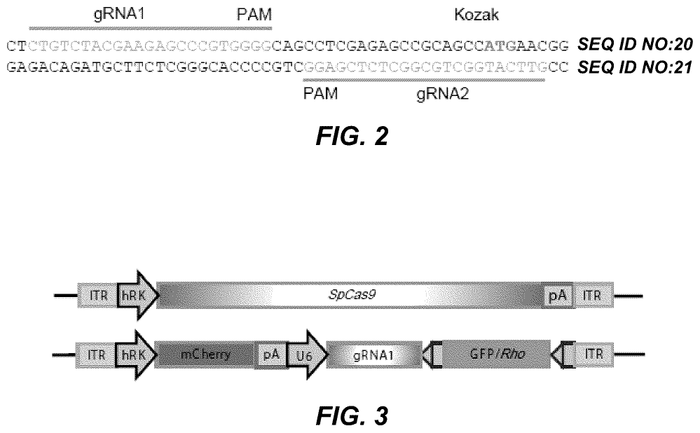

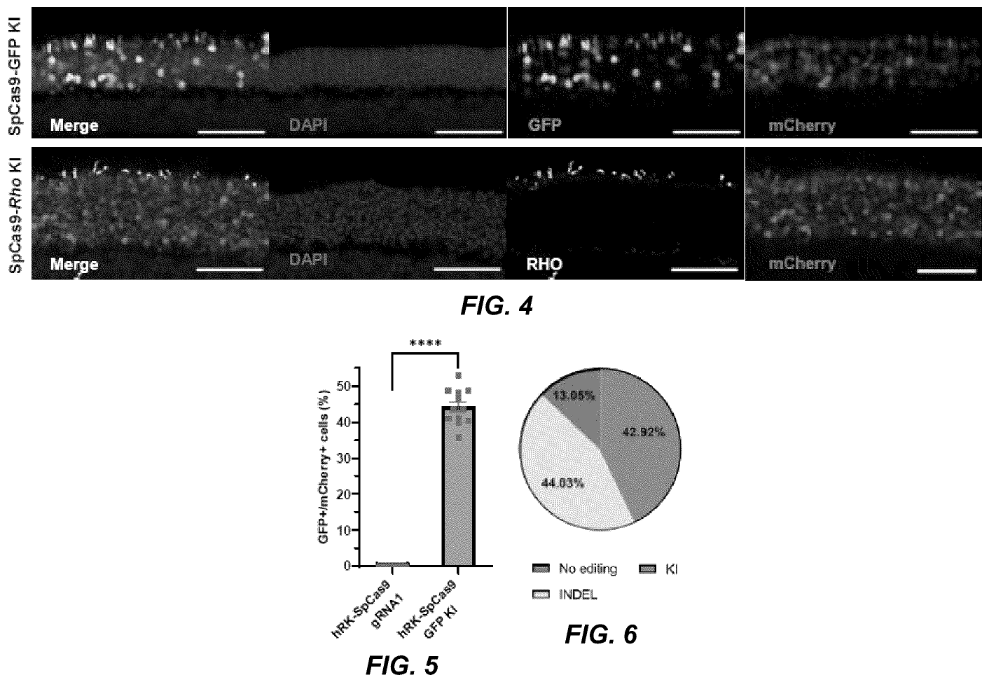

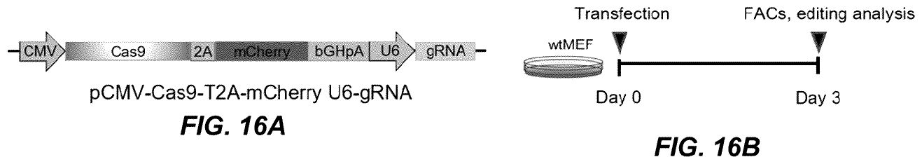

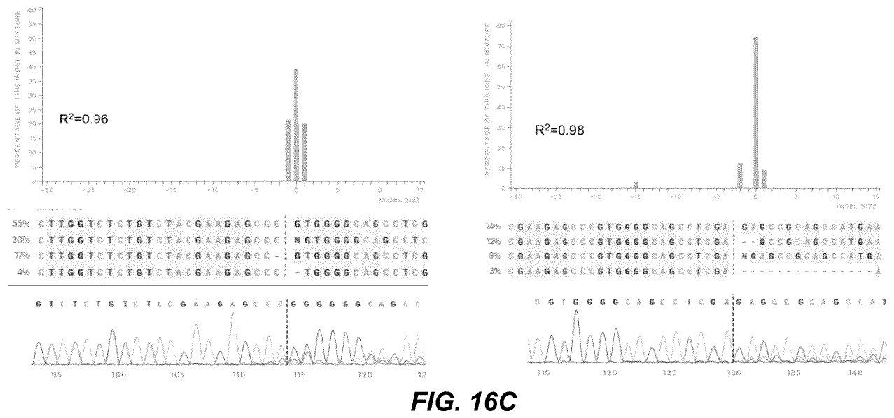

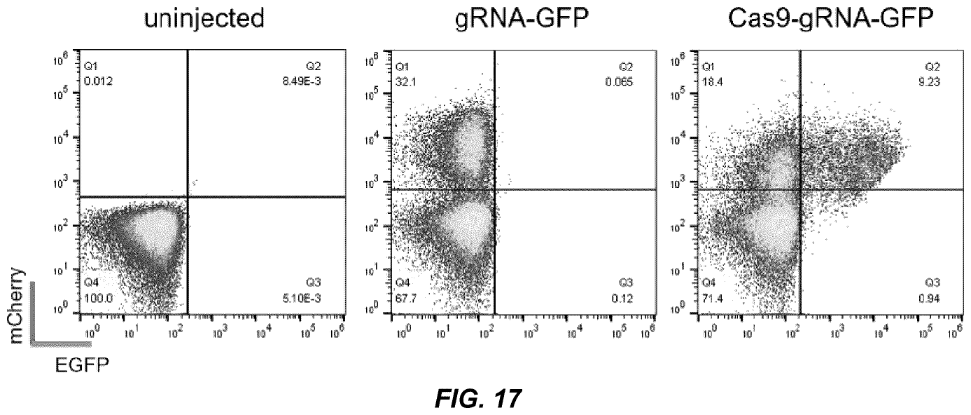

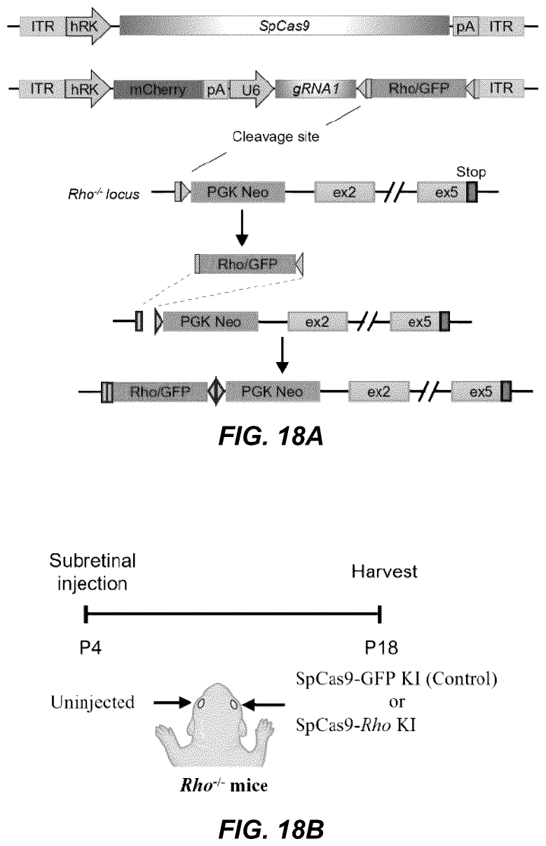

Described herein is a mutation-independent gene editing strategy to treat RHO-associated adRP. Briefly, AAV-Cas9-mediated gene knock-in (KI) was targeted to the 5′ untranslated region (UTR) of the RHO gene by homology-independent targeted integration (HITI) (FIG. 1). 4 Two gRNAs of SpCas9 that are upstream of the Kozak sequence of the mouse RHO gene were chosen (FIG. 2; comprised in SEQ ID NOs:20 and 21). The gRNA (gRNA1; comprised in SEQ ID NO:20) with higher cutting efficiency (FIG. 16) was used for in vivo tests. To ensure fitting of SpCas9 in the AAV vector, a short hRK promoter was used to drive SpCas9 expression in the photoreceptor cells (FIG. 3). hRK-SpCas9 was packaged in a first AAV vector, and hRK-mCherry, pU6-gRNA, and a HITI donor sequence were packaged in a second AAV vector (FIG. 3). The two vectors were subretinally injected into neonatal mouse eyes, and ˜60% rods (32% total retinal cells) were infected by AAV, as indicated by mCherry expression (FIG. 17). HITI AAV-mediated GFP KI efficiency was as high as 45% in the infected photoreceptors in vivo (FIGS. 4-6), and RHO KI was confirmed by RHO staining in the RHO−/− 1 mice,⁵ which retain the gRNA1 target site in the RHO 5′ UTR (FIG. 4, FIG. 18). Next generation sequencing (NGS) results showed that the in vivo KI efficiency was as high as 43%, with a small INDEL rate 44%, and unmodified alleles representing only 13% (FIG. 6). Together, these results demonstrate that, in some aspects, an HITI-AAV approach can mediate efficient gene KI into RHO 5′ UTR.

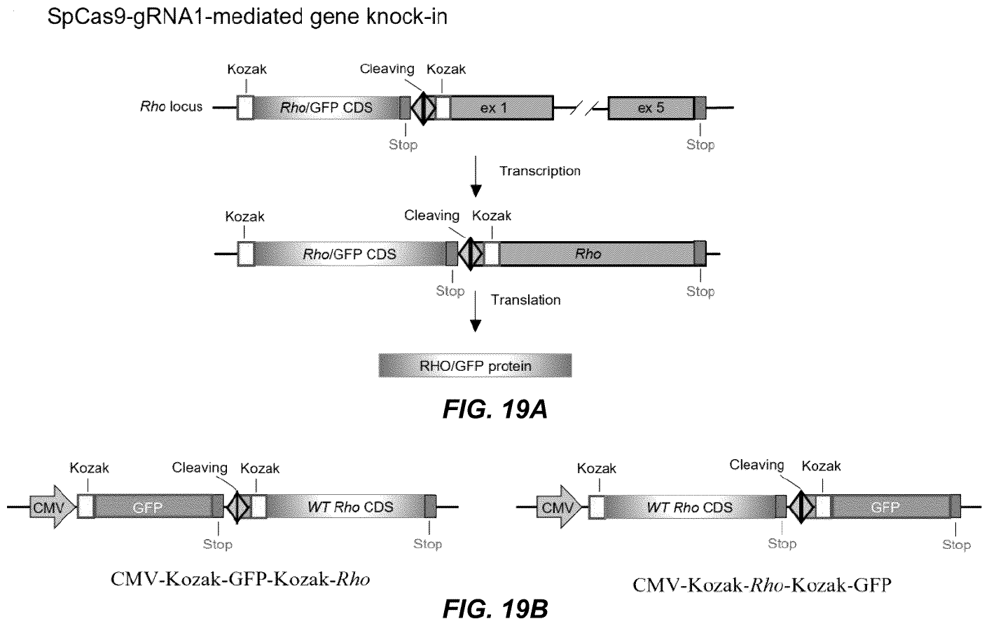

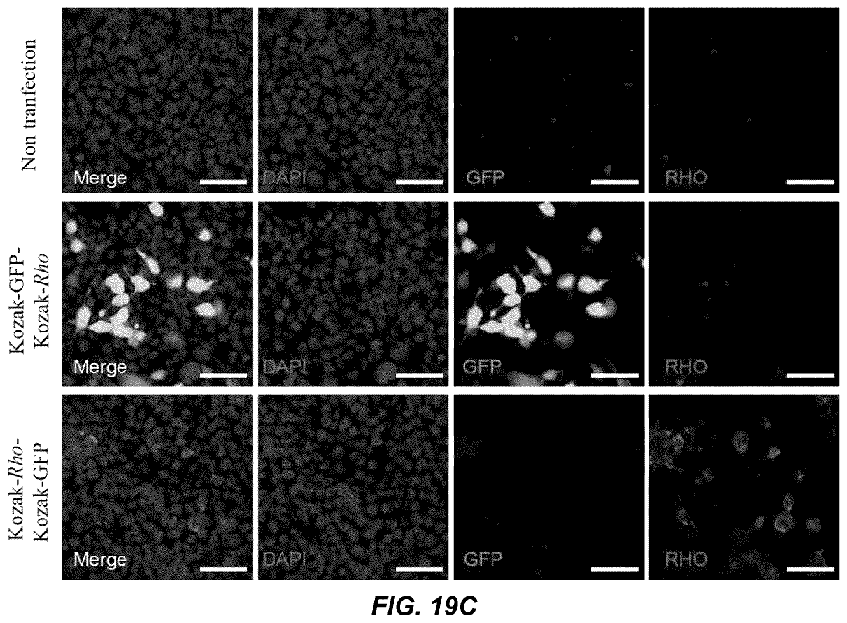

In some aspects, the endogenous RHO P23H allele, following the inserted wild-type RHO coding sequence, retains an intact translation initiation element (e.g., Kozak sequence), and the toxic mutated RHO protein may continue to be expressed. Accordingly, expression of the endogenous gene was evaluated by cloning a reporter plasmid by synthesizing the RHO genomic sequence after integration of GFP. The expression of the Kozak-GFP-STOP-Kozak-RHO construct demonstrated that GFP protein is translated, while RHO protein translation is inhibited (FIG. 19). Without wishing to be bound by theory, in some aspects, inclusion of a STOP codon within the 5′ UTR of the integrated gene can signal a halt to native protein translation in cells.

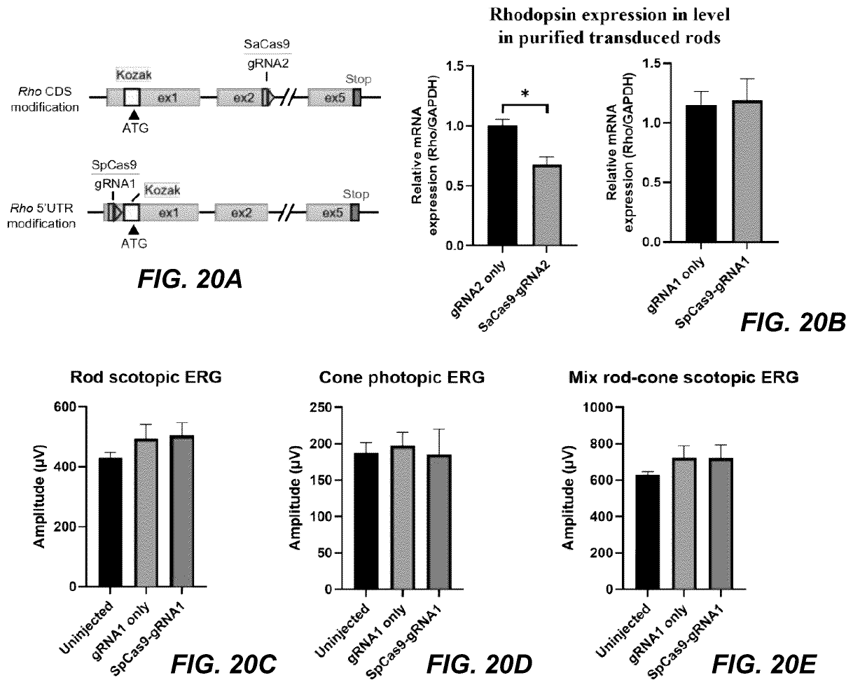

In some aspects, insertions and/or deletions (INDELs) near the translation start site can lead to lower levels of RHO expression and thereby affect visual function. Accordingly, the impacts of INDELs in the RHO locus on RHO expression levels and mouse visual function was assessed. C57BL/6 mice were injected with dual vectors without an HITI donor to create INDELs. mCherry+ photoreceptors were isolated for RHO mRNA-level examination. qPCR results showed that RHO mRNA was not significantly affected by 5′ UTR KI, in contrast to a significant decrease in expression by CDS KI (FIG. 20). Visual function tested by the optomotor assay and electroretinography (ERG) showed that visual acuity and light-induced potential change were not affected in the injected group. Thus, in some aspects, INDELs in 5′ UTR do not affect endogenous RHO gene expression or visual function.

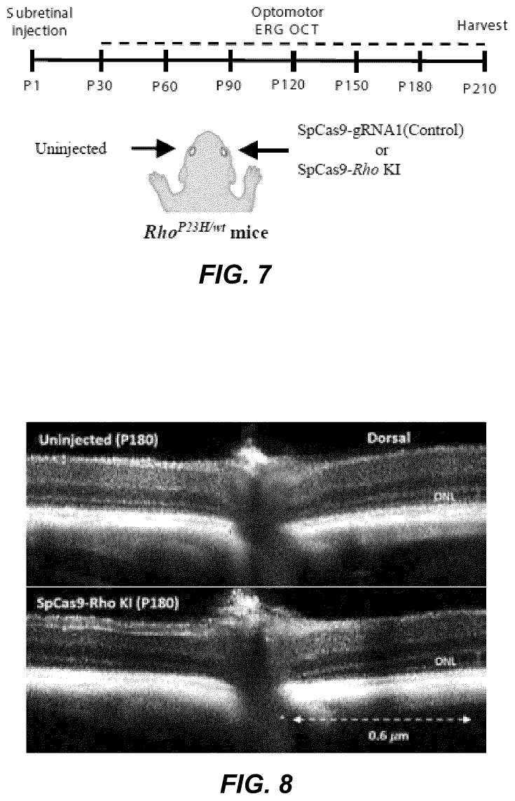

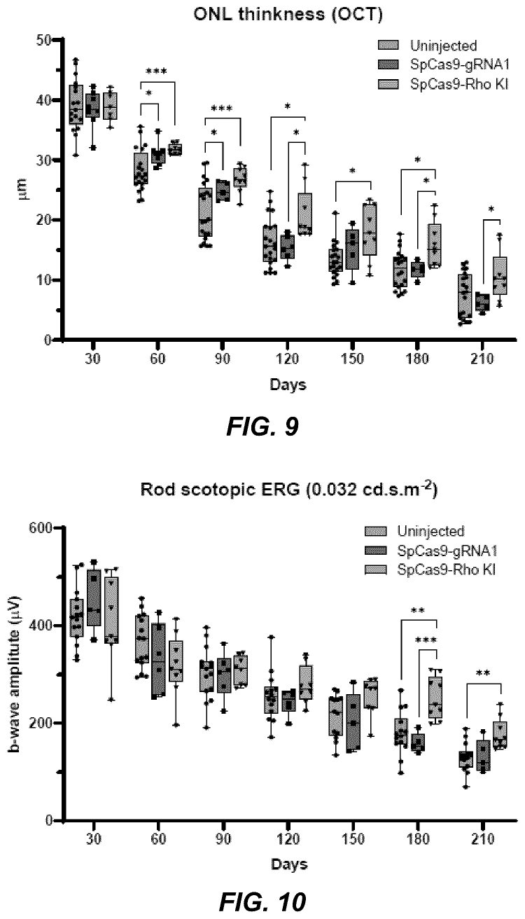

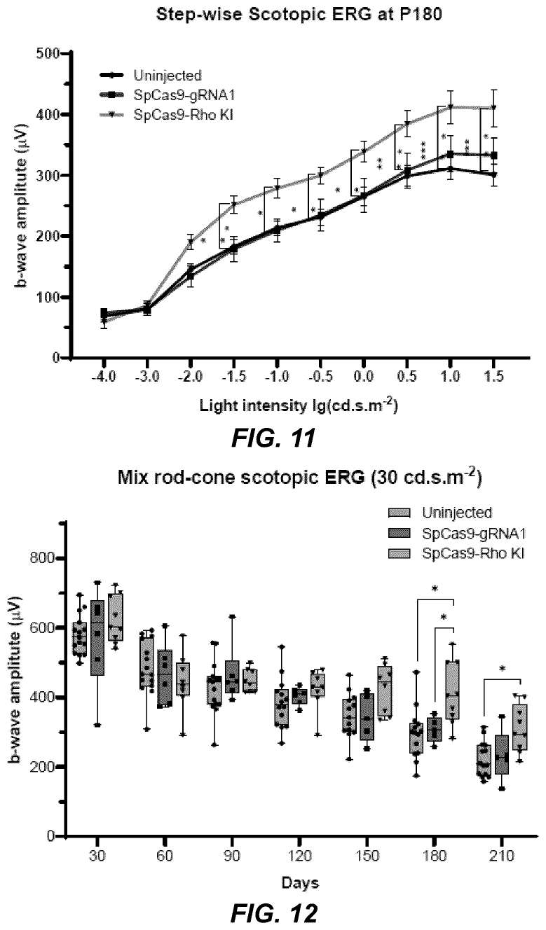

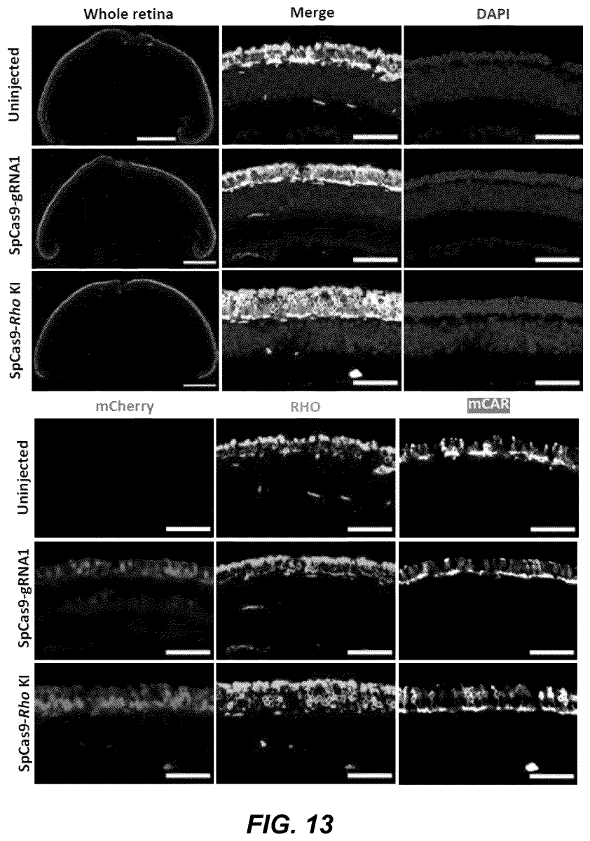

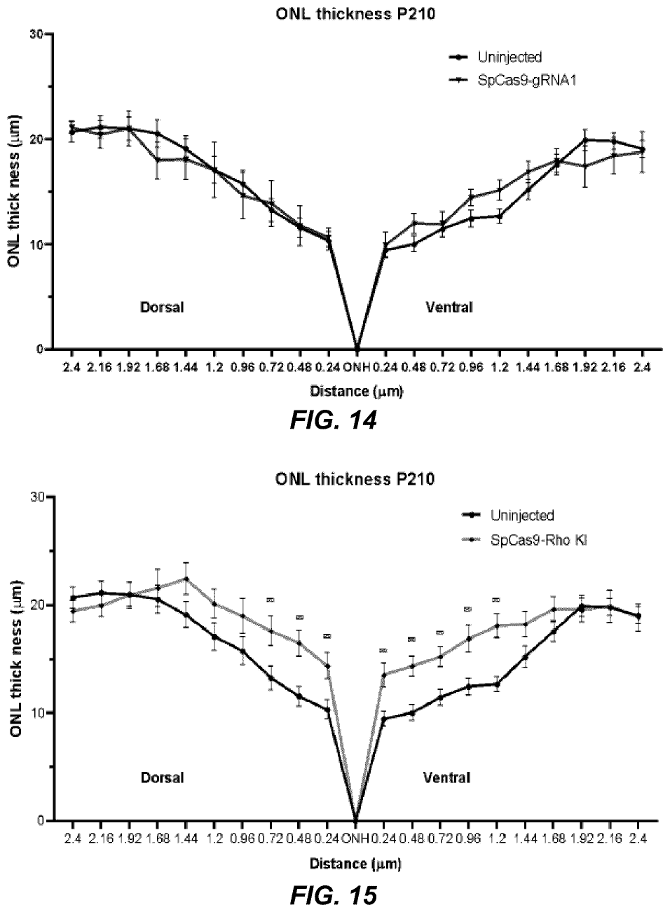

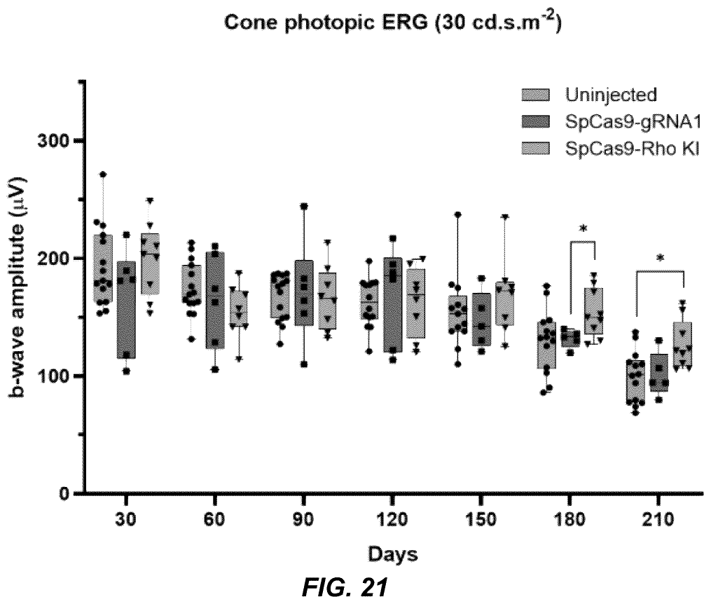

To test the therapeutic effect of 5′ UTR RHO KI, RHO^{P23H/wt} mice, which have a similar progressive photoreceptor degeneration as human patients with the same mutation, were treated with the 2 dual AAV vectors on P1 (FIG. 7). Monthly retina structure examination by optical coherent tomography (OCT) revealed significant increases of photoreceptor layer thickness from P60 to P210 in the RHO KI group in comparison to the control groups (FIGS. 8-9). The ERG results showed that RHO KI eyes have significantly higher scotopic ERG B-wave amplitude under dim light condition (0.032 cd·s·m⁻²) from P180 to P210, suggesting a better light-sensing function of the rods (FIGS. 8-11). Both rod-cone mixed scotopic ERG and cone-dominant photopic ERG showed increased amplitude with RHO KI at late stages (FIG. 10, FIG. 21). At the endpoint of the experiment, histological analysis of the harvested eyes showed that RHO KI treatment better preserved photoreceptor cell layer thickness with more Rhodopsin+ rods and better cone morphology (FIGS. 11-13). In all assays, the control treatment without HITI RHO donor did not provide any beneficial effect or induce any toxic effect in RHO^{P23H/wt} mice (FIGS. 9-12), in line with the results in the wild type mice (FIG. 20). Together, these results show that, in some aspects, AAV-mediated RHO KI into the 5′ UTR in an allele-independent manner can efficiently hamper rod degeneration and vision loss in RHo^{P23H/wt} mice.

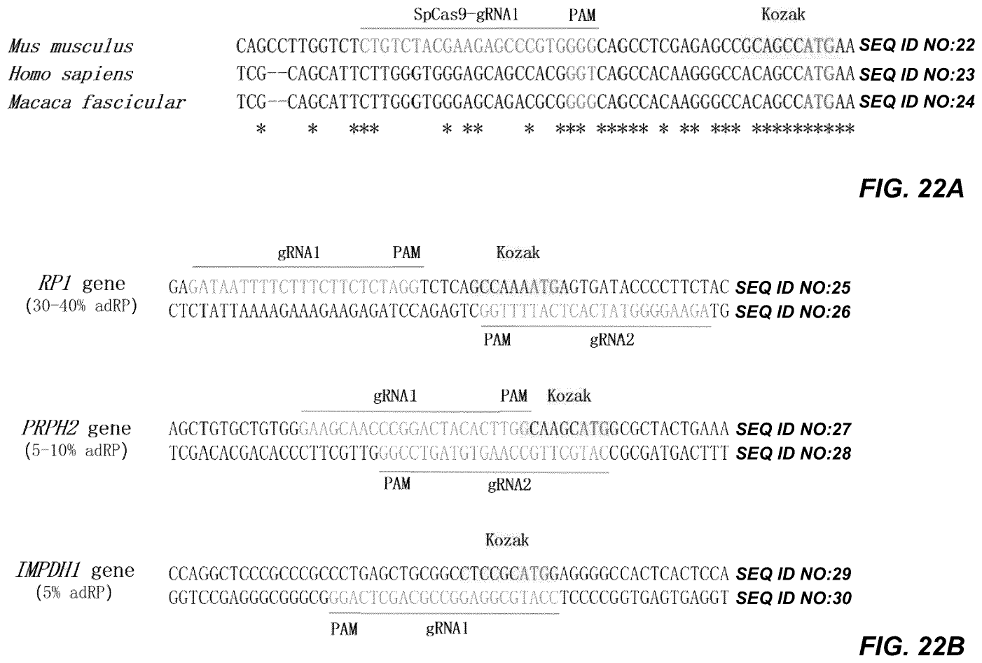

Recently, AAV-HITI-mediated gene KI targeting of disease gene CDS was shown in both mouse retina and liver. 5 As shown herein, in some aspects, gene KI targeting of the 5′ UTR, rather than the CDS, is more efficacious and safer. First, in some aspects, unlike KI targeting of a CDS, the exogenous gene sequence inserted into the 5′ UTR, which contains a translation initiation element, can but is not required to be in-frame with the endogenous CDS. Second, in some aspects, the STOP codon of the inserted exogenous gene in the 5′ UTR can block expression of the downstream endogenous gene, while CDS KI may lead to the expression of truncated proteins which may function in a toxic dominant-negative manner. Additionally, the 5′ UTR INDELs do not abolish endogenous gene expression in the cells without successful KI, while CDS INDELs may create a reading frame shift and a disadvantageous knockout effect of the wild-type allele. PAM sites of various Cas9 enzymes are readily available and conserved in the sequence upstream of translation initiation elements (e.g., Kozak sequences) in the 5′ UTR of the most common adRP disease genes (FIG. 22, SEQ ID NOs:22-30), suggesting that 5′ UTR gene KI may be widely applicable.

Altogether, disclosed herein is a novel mutation-independent gene KI approach that targets the 5′ UTR of both alleles of a disease gene that, in some aspects, has therapeutic potential in treating allele-dominant diseases.

### Example 2—Exemplary Methods

Animals. RHO P23″2314 and C57BL/6J mice were purchased from The Jackson Laboratory. RHO^{-/-} mice were obtained from Janis Lem (Tufts University, Boston, Massachusetts, USA).¹ RHO^{P23H/wt}, RHO^{P23H/P23H} and RHO^{-/-} mice were genotyped by PCR with a primer set suggested by earlier publications.^{1,2} All mice were kept on a 12h light/12h dark cycle.

Plasmid construction. pAAV-CMV-SpCas9-2A-mCherry-bGHPA-U6-gRNA for SpCas9 gRNA knock-out efficiency testing was constructed as previously described³. Briefly, the SaCas9 cassette of pAAV-CMV-SaCas9-U6-Bsal-gRNA (Addgene No. 61591) was replaced by SpCas9, which was amplified from LentiV-Cas9-puro (Addgene No. 108100), via AgeI and BamHI sites. gRNA1 and gRNA2 (primer sets Rho gRNA1/2 Sp F and R) were inserted into pAAV-CMV-SpCas9-2A-mCherry-bGHPA-U6-gRNA via a BsaI site.

For testing the gene knock-in therapy, pAAV-hRK-SpCas9 (SEQ ID NO:31), pAAV-hRK-mCherry-U6-gRNA1 (SEQ ID NO:32), pAAV-hRK-mCherry-U6-gRNA1-RHO-HITI donor (SEQ ID NO:33), and pAAV-hRK-mCherry-U6-gRNA1-GFP-HITI donor (SEQ ID NO:34) were constructed. pAAV-hRK-SpCas9 was made by replacing the iZsGreen in pAAV-hRK-iZsGreen (T. Li Laboratory, National Eye Institute, Bethesda, MD 4) with the SpCas9 sequence. pAAV-hRK-mCherry-U6-gRNA1 was cloned by inserting hRK-mCherry via Xbal and BamHI first and inserting gRNA1 sequence via BsaI site next into the backbone plasmid pAAV-CMV-SaCas9-U6-Bsal-gRNA (Addgene No. 61591). To construct the RHO-HITI donor, RHO-CDS was amplified from the retinal cDNA of C57BL/6J with primers containing CRISPR/Cas9 gRNA1 targeted site (RHO CDS F and R) and inserted into the backbone vector pAAV-hRK-mCherry-pA-U6-gRNA1 to generate pAAV-hRK-mCherry-pA-U6-gRNA1-RHO HITI donor. pAAV-hRK-mCherry-pA-U6-gRNA-GFP HITI donor was constructed in a similar method.

The nucleotide sequences of plasmids for gene knock-in therapy are provided as follows:

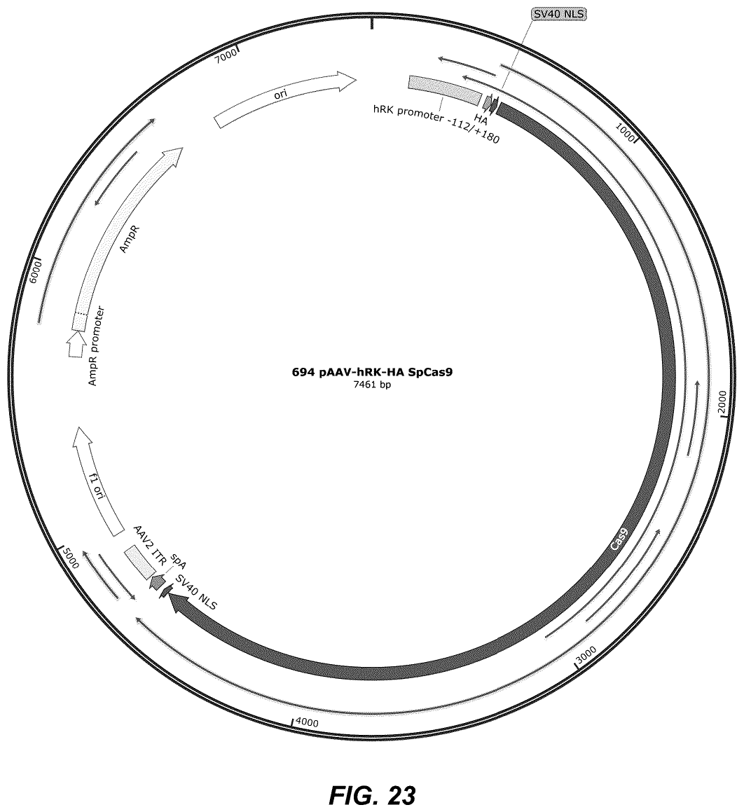

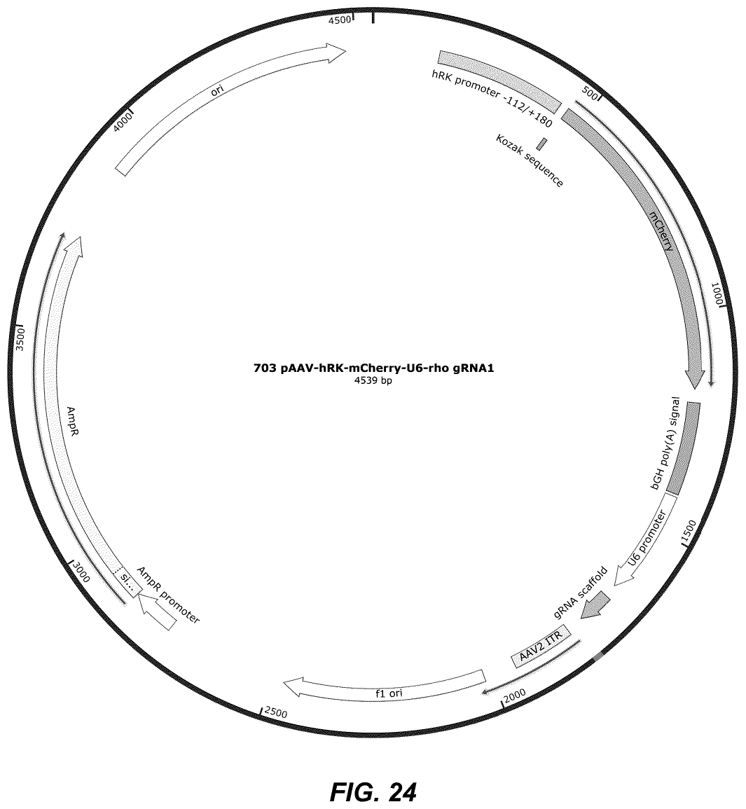

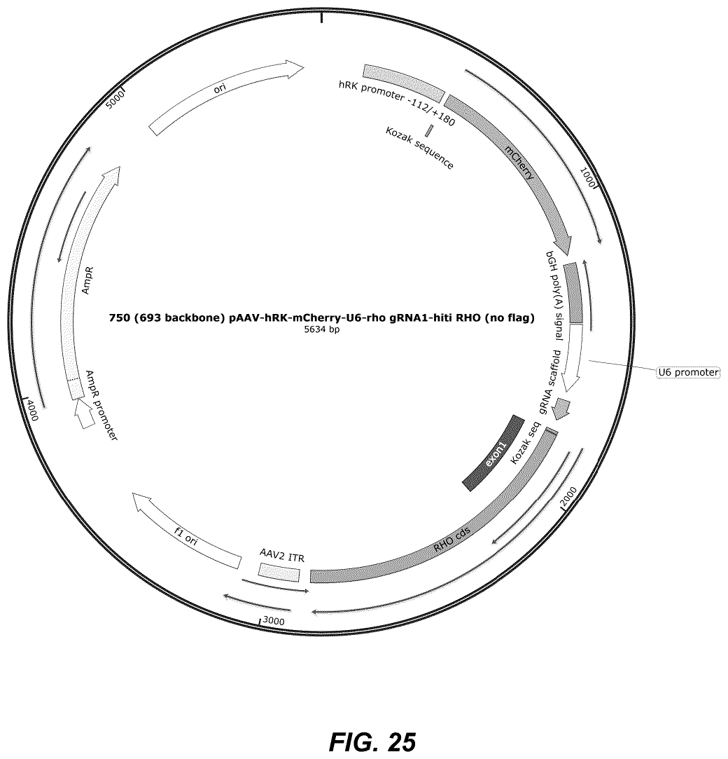

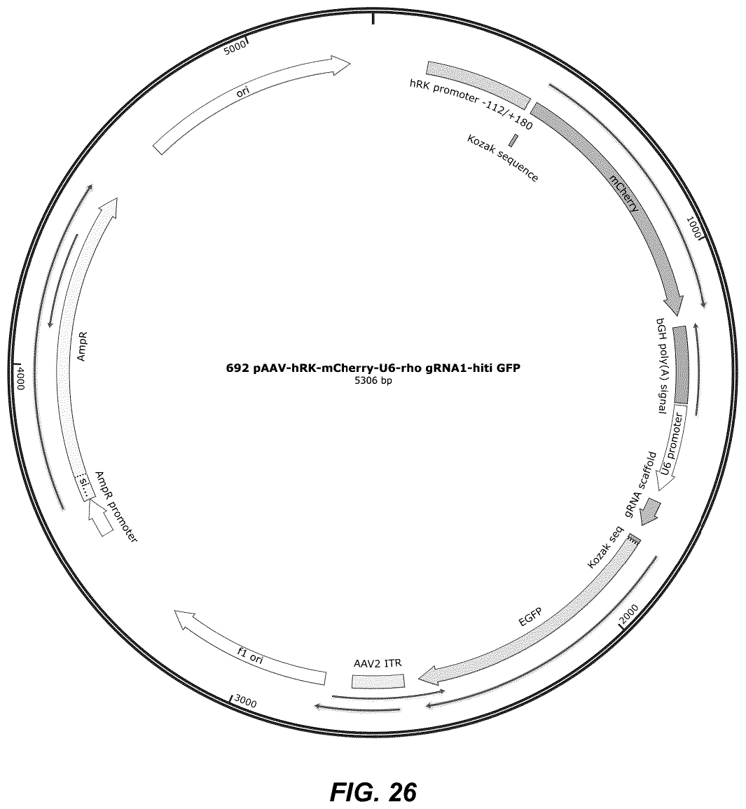

pAAV-hRK-SpCas9:
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CTCTAGACTCGAGTTGGGCCCCAGAAGCCTGGTGGTTGTTTGTCC
TTCTCAGGGGAAAAGTGAGGCGGCCCCTTGGAGGAAGGGGCCGGG
CAGAATGATCTAATCGGATTCCAAGCAGCTCAGGGGATTGTCTTT
TTCTAGCACCTTCTTGCCACTCCTAAGCGTCCTCCGTGACCCCGG
CTGGGATTTAGCCTGGTGCTGTGTCAGCCCCGGTCTCCCAGGGGC
TTCCCAGTGGTCCCCAGGAACCCTCGACAGGGCCCGGTCTCTCTC
GTCCAGCAAGGGCAGGGACGGGCCACAGGCCAAGGGCCCTCGATA
CCGGTGCCACCATGTACCCATACGATGTTCCAGATTACGCTTCGC
CGAAGAAAAAGCGCAAGGTCGAAGCGTCCGACAAGAAGTACAGCA
TCGGCCTGGACATCGGCACCAACTCTGTGGGCTGGGCCGTGATCA
CCGACGAGTACAAGGTGCCCAGCAAGAAATTCAAGGTGCTGGGCA
ACACCGACCGGCACAGCATCAAGAAGAACCTGATCGGAGCCCTGC
TGTTCGACAGCGGCGAAACAGCCGAGGCCACCCGGCTGAAGAGAA
CCGCCAGAAGAAGATACACCAGACGGAAGAACCGGATCTGCTATC
TGCAAGAGATCTTCAGCAACGAGATGGCCAAGGTGGACGACAGCT
TCTTCCACAGACTGGAAGAGTCCTTCCTGGTGGAAGAGGATAAGA
AGCACGAGCGGCACCCCATCTTCGGCAACATCGTGGACGAGGTGG
CCTACCACGAGAAGTACCCCACCATCTACCACCTGAGAAAGAAAC
TGGTGGACAGCACCGACAAGGCCGACCTGCGGCTGATCTATCTGG
CCCTGGCCCACATGATCAAGTTCCGGGGCCACTTCCTGATCGAGG
GCGACCTGAACCCCGACAACAGCGACGTGGACAAGCTGTTCATCC
AGCTGGTGCAGACCTACAACCAGCTGTTCGAGGAAAACCCCATCA
ACGCCAGCGGCGTGGACGCCAAGGCCATCCTGTCTGCCAGACTGA
GCAAGAGCAGACGGCTGGAAAATCTGATCGCCCAGCTGCCCGGCG
AGAAGAAGAATGGCCTGTTCGGCAACCTGATTGCCCTGAGCCTGG
GCCTGACCCCCAACTTCAAGAGCAACTTCGACCTGGCCGAGGATG
CCAAACTGCAGCTGAGCAAGGACACCTACGACGACGACCTGGACA
ACCTGCTGGCCCAGATCGGCGACCAGTACGCCGACCTGTTTCTGG
CCGCCAAGAACCTGTCCGACGCCATCCTGCTGAGCGACATCCTGA
GAGTGAACACCGAGATCACCAAGGCCCCCCTGAGCGCCTCTATGA
TCAAGAGATACGACGAGCACCACCAGGACCTGACCCTGCTGAAAG
CTCTCGTGCGGCAGCAGCTGCCTGAGAAGTACAAAGAGATTTTCT
TCGACCAGAGCAAGAACGGCTACGCCGGCTACATTGACGGCGGAG
CCAGCCAGGAAGAGTTCTACAAGTTCATCAAGCCCATCCTGGAAA
AGATGGACGGCACCGAGGAACTGCTCGTGAAGCTGAACAGAGAGG
ACCTGCTGCGGAAGCAGCGGACCTTCGACAACGGCAGCATCCCCC
ACCAGATCCACCTGGGAGAGCTGCACGCCATTCTGCGGCGGCAGG
AAGATTTTTACCCATTCCTGAAGGACAACCGGGAAAAGATCGAGA
AGATCCTGACCTTCCGCATCCCCTACTACGTGGGCCCTCTGGCCA
GGGGAAACAGCAGATTCGCCTGGATGACCAGAAAGAGCGAGGAAA
CCATCACCCCCTGGAACTTCGAGGAAGTGGTGGACAAGGGCGCTT
CCGCCCAGAGCTTCATCGAGCGGATGACCAACTTCGATAAGAACC
TGCCCAACGAGAAGGTGCTGCCCAAGCACAGCCTGCTGTACGAGT
ACTTCACCGTGTATAACGAGCTGACCAAAGTGAAATACGTGACCG
AGGGAATGAGAAAGCCCGCCTTCCTGAGCGGCGAGCAGAAAAAGG
CCATCGTGGACCTGCTGTTCAAGACCAACCGGAAAGTGACCGTGA
AGCAGCTGAAAGAGGACTACTTCAAGAAAATCGAGTGCTTCGACT
CCGTGGAAATCTCCGGCGTGGAAGATCGGTTCAACGCCTCCCTGG
GCACATACCACGATCTGCTGAAAATTATCAAGGACAAGGACTTCC
TGGACAATGAGGAAAACGAGGACATTCTGGAAGATATCGTGCTGA
CCCTGACACTGTTTGAGGACAGAGAGATGATCGAGGAACGGCTGA
AAACCTATGCCCACCTGTTCGACGACAAAGTGATGAAGCAGCTGA
AGCGGCGGAGATACACCGGCTGGGGCAGGCTGAGCCGGAAGCTGA
TCAACGGCATCCGGGACAAGCAGTCCGGCAAGACAATCCTGGATT
TCCTGAAGTCCGACGGCTTCGCCAACAGAAACTTCATGCAGCTGA
TCCACGACGACAGCCTGACCTTTAAAGAGGACATCCAGAAAGCCC
AGGTGTCCGGCCAGGGCGATAGCCTGCACGAGCACATTGCCAATC
TGGCCGGCAGCCCCGCCATTAAGAAGGGCATCCTGCAGACAGTGA
AGGTGGTGGACGAGCTCGTGAAAGTGATGGGCCGGCACAAGCCCG
AGAACATCGTGATCGAAATGGCCAGAGAGAACCAGACCACCCAGA
AGGGACAGAAGAACAGCCGCGAGAGAATGAAGCGGATCGAAGAGG
GCATCAAAGAGCTGGGCAGCCAGATCCTGAAAGAACACCCCGTGG
AAAACACCCAGCTGCAGAACGAGAAGCTGTACCTGTACTACCTGC
AGAATGGGCGGGATATGTACGTGGACCAGGAACTGGACATCAACC
GGCTGTCCGACTACGATGTGGACCATATCGTGCCTCAGAGCTTTC
TGAAGGACGACTCCATCGACAACAAGGTGCTGACCAGAAGCGACA
AGAACCGGGGCAAGAGCGACAACGTGCCCTCCGAAGAGGTCGTGA
AGAAGATGAAGAACTACTGGCGGCAGCTGCTGAACGCCAAGCTGA
TTACCCAGAGAAAGTTCGACAATCTGACCAAGGCCGAGAGAGGCG
GCCTGAGCGAACTGGATAAGGCCGGCTTCATCAAGAGACAGCTGG
TGGAAACCCGGCAGATCACAAAGCACGTGGCACAGATCCTGGACT
CCCGGATGAACACTAAGTACGACGAGAATGACAAGCTGATCCGGG
AAGTGAAAGTGATCACCCTGAAGTCCAAGCTGGTGTCCGATTTCC
GGAAGGATTTCCAGTTTTACAAAGTGCGCGAGATCAACAACTACC
ACCACGCCCACGACGCCTACCTGAACGCCGTCGTGGGAACCGCCC
TGATCAAAAAGTACCCTAAGCTGGAAAGCGAGTTCGTGTACGGCG
ACTACAAGGTGTACGACGTGCGGAAGATGATCGCCAAGAGCGAGC
AGGAAATCGGCAAGGCTACCGCCAAGTACTTCTTCTACAGCAACA
TCATGAACTTTTTCAAGACCGAGATTACCCTGGCCAACGGCGAGA
TCCGGAAGCGGCCTCTGATCGAGACAAACGGCGAAACCGGGGAGA
TCGTGTGGGATAAGGGCCGGGATTTTGCCACCGTGCGGAAAGTGC
TGAGCATGCCCCAAGTGAATATCGTGAAAAAGACCGAGGTGCAGA
CAGGCGGCTTCAGCAAAGAGTCTATCCTGCCCAAGAGGAACAGCG
ATAAGCTGATCGCCAGAAAGAAGGACTGGGACCCTAAGAAGTACG
GCGGCTTCGACAGCCCCACCGTGGCCTATTCTGTGCTGGTGGTGG
CCAAAGTGGAAAAGGGCAAGTCCAAGAAACTGAAGAGTGTGAAAG
AGCTGCTGGGGATCACCATCATGGAAAGAAGCAGCTTCGAGAAGA
ATCCCATCGACTTTCTGGAAGCCAAGGGCTACAAAGAAGTGAAAA
AGGACCTGATCATCAAGCTGCCTAAGTACTCCCTGTTCGAGCTGG
AAAACGGCCGGAAGAGAATGCTGGCCTCTGCCGGCGAACTGCAGA
AGGGAAACGAACTGGCCCTGCCCTCCAAATATGTGAACTTCCTGT
ACCTGGCCAGCCACTATGAGAAGCTGAAGGGCTCCCCCGAGGATA
ATGAGCAGAAACAGCTGTTTGTGGAACAGCACAAGCACTACCTGG
ACGAGATCATCGAGCAGATCAGCGAGTTCTCCAAGAGAGTGATCC
TGGCCGACGCTAATCTGGACAAAGTGCTGTCCGCCTACAACAAGC
ACCGGGATAAGCCCATCAGAGAGCAGGCCGAGAATATCATCCACC
TGTTTACCCTGACCAATCTGGGAGCCCCTGCCGCCTTCAAGTACT
TTGACACCACCATCGACCGGAAGAGGTACACCAGCACCAAAGAGG
TGCTGGACGCCACCCTGATCCACCAGAGCATCACCGGCCTGTACG
AGACACGGATCGACCTGTCTCAGCTGGGAGGCGACAGCCCCAAGA
AGAAGAGAAAGGTGGAGGCCAGCTAAGAATTCAATAAAAGATCTT
TATTTTCATTAGATCTGTGTGTTGGTTTTTTGTGTGCGGCCGCAG
GAACCCCTAGTGATGGAGTTGGCCACTCCCTCTCTGCGCGCTCGC
TCGCTCACTGAGGCCGGGCGACCAAAGGTCGCCCGACGCCCGGGC
TTTGCCCGGGCGGCCTCAGTGAGCGAGCGAGCGCGCAGCTGCCTG
CAGGGGCGCCTGATGCGGTATTTTCTCCTTACGCATCTGTGCGGT
ATTTCACACCGCATACGTCAAAGCAACCATAGTACGCGCCCTGTA
GCGGCGCATTAAGCGCGGCGGGTGTGGTGGTTACGCGCAGCGTGA
CCGCTACACTTGCCAGCGCCTTAGCGCCCGCTCCTTTCGCTTTCT
TCCCTTCCTTTCTCGCCACGTTCGCCGGCTTTCCCCGTCAAGCTC
TAAATCGGGGGCTCCCTTTAGGGTTCCGATTTAGTGCTTTACGGC
ACCTCGACCCCAAAAAACTTGATTTGGGTGATGGTTCACGTAGTG
GGCCATCGCCCTGATAGACGGTTTTTCGCCCTTTGACGTTGGAGT
CCACGTTCTTTAATAGTGGACTCTTGTTCCAAACTGGAACAACAC
TCAACTCTATCTCGGGCTATTCTTTTGATTTATAAGGGATTTTGC
CGATTTCGGTCTATTGGTTAAAAAATGAGCTGATTTAACAAAAAT
TTAACGCGAATTTTAACAAAATATTAACGTTTACAATTTTATGGT
GCACTCTCAGTACAATCTGCTCTGATGCCGCATAGTTAAGCCAGC
CCCGACACCCGCCAACACCCGCTGACGCGCCCTGACGGGCTTGTC
TGCTCCCGGCATCCGCTTACAGACAAGCTGTGACCGTCTCCGGGA
GCTGCATGTGTCAGAGGTTTTCACCGTCATCACCGAAACGCGCGA
GACGAAAGGGCCTCGTGATACGCCTATTTTTATAGGTTAATGTCA
TGATAATAATGGTTTCTTAGACGTCAGGTGGCACTTTTCGGGGAA
ATGTGCGCGGAACCCCTATTTGTTTATTTTTCTAAATACATTCAA
ATATGTATCCGCTCATGAGACAATAACCCTGATAAATGCTTCAAT
AATATTGAAAAAGGAAGAGTATGAGTATTCAACATTTCCGTGTCG
CCCTTATTCCCTTTTTTGCGGCATTTTGCCTTCCTGTTTTTGCTC
ACCCAGAAACGCTGGTGAAAGTAAAAGATGCTGAAGATCAGTTGG
GTGCACGAGTGGGTTACATCGAACTGGATCTCAACAGCGGTAAGA
TCCTTGAGAGTTTTCGCCCCGAAGAACGTTTTCCAATGATGAGCA
CTTTTAAAGTTCTGCTATGTGGCGCGGTATTATCCCGTATTGACG
CCGGGCAAGAGCAACTCGGTCGCCGCATACACTATTCTCAGAATG
ACTTGGTTGAGTACTCACCAGTCACAGAAAAGCATCTTACGGATG
GCATGACAGTAAGAGAATTATGCAGTGCTGCCATAACCATGAGTG
ATAACACTGCGGCCAACTTACTTCTGACAACGATCGGAGGACCGA
AGGAGCTAACCGCTTTTTTGCACAACATGGGGGATCATGTAACTC
GCCTTGATCGTTGGGAACCGGAGCTGAATGAAGCCATACCAAACG
ACGAGCGTGACACCACGATGCCTGTAGCAATGGCAACAACGTTGC
GCAAACTATTAACTGGCGAACTACTTACTCTAGCTTCCCGGCAAC
AATTAATAGACTGGATGGAGGCGGATAAAGTTGCAGGACCACTTC
TGCGCTCGGCCCTTCCGGCTGGCTGGTTTATTGCTGATAAATCTG
GAGCCGGTGAGCGTGGAAGCCGCGGTATCATTGCAGCACTGGGGC
CAGATGGTAAGCCCTCCCGTATCGTAGTTATCTACACGACGGGGA
GTCAGGCAACTATGGATGAACGAAATAGACAGATCGCTGAGATAG
GTGCCTCACTGATTAAGCATTGGTAACTGTCAGACCAAGTTTACT
CATATATACTTTAGATTGATTTAAAACTTCATTTTTAATTTAAAA
GGATCTAGGTGAAGATCCTTTTTGATAATCTCATGACCAAAATCC
CTTAACGTGAGTTTTCGTTCCACTGAGCGTCAGACCCCGTAGAAA
AGATCAAAGGATCTTCTTGAGATCCTTTTTTTCTGCGCGTAATCT
GCTGCTTGCAAACAAAAAAACCACCGCTACCAGCGGTGGTTTGTT
TGCCGGATCAAGAGCTACCAACTCTTTTTCCGAAGGTAACTGGCT
TCAGCAGAGCGCAGATACCAAATACTGTTCTTCTAGTGTAGCCGT
AGTTAGGCCACCACTTCAAGAACTCTGTAGCACCGCCTACATACC
TCGCTCTGCTAATCCTGTTACCAGTGGCTGCTGCCAGTGGCGATA
AGTCGTGTCTTACCGGGTTGGACTCAAGACGATAGTTACCGGATA
AGGCGCAGCGGTCGGGCTGAACGGGGGGTTCGTGCACACAGCCCA
GCTTGGAGCGAACGACCTACACCGAACTGAGATACCTACAGCGTG
AGCTATGAGAAAGCGCCACGCTTCCCGAAGGGAGAAAGGCGGACA
GGTATCCGGTAAGCGGCAGGGTCGGAACAGGAGAGCGCACGAGGG
AGCTTCCAGGGGGAAACGCCTGGTATCTTTATAGTCCTGTCGGGT
TTCGCCACCTCTGACTTGAGCGTCGATTTTTGTGATGCTCGTCAG
GGGGGCGGAGCCTATGGAAAAACGCCAGCAACGCGGCCTTTTTAC
GGTTCCTGGCCTTTTGCTGGCCTTTTGCTCACATGT
(SEQ ID NO: 31; see FIG. 23 for a plasmid map).
pAAV-hRK-mCherry-U6-gRNA1:
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CTCTAGACTCGAGTTGGGCCCCAGAAGCCTGGTGGTTGTTTGTCC
TTCTCAGGGGAAAAGTGAGGCGGCCCCTTGGAGGAAGGGGCCGGG
CAGAATGATCTAATCGGATTCCAAGCAGCTCAGGGGATTGTCTTT
TTCTAGCACCTTCTTGCCACTCCTAAGCGTCCTCCGTGACCCCGG
CTGGGATTTAGCCTGGTGCTGTGTCAGCCCCGGTCTCCCAGGGGC
TTCCCAGTGGTCCCCAGGAACCCTCGACAGGGCCCGGTCTCTCTC
GTCCAGCAAGGGCAGGGACGGGCCACAGGCCAAGGGCCCTCGATA
CCGGTGCCACCATGGTGAGCAAGGGCGAGGAGGATAACATGGCCA
TCATCAAGGAGTTCATGCGCTTCAAGGTGCACATGGAGGGCTCCG
TGAACGGCCACGAGTTCGAGATCGAGGGCGAGGGCGAGGGCCGCC
CCTACGAGGGCACCCAGACCGCCAAGCTGAAGGTGACCAAGGGTG
GCCCCCTGCCCTTCGCCTGGGACATCCTGTCCCCTCAGTTCATGT
ACGGCTCCAAGGCCTACGTGAAGCACCCCGCCGACATCCCCGACT
ACTTGAAGCTGTCCTTCCCCGAGGGCTTCAAGTGGGAGCGCGTGA
TGAACTTCGAGGACGGCGGCGTGGTGACCGTGACCCAGGACTCCT
CCCTGCAGGACGGCGAGTTCATCTACAAGGTGAAGCTGCGCGGCA
CCAACTTCCCCTCCGACGGCCCCGTAATGCAGAAGAAGACCATGG
GCTGGGAGGCCTCCTCCGAGCGGATGTACCCCGAGGACGGCGCCC
TGAAGGGCGAGATCAAGCAGAGGCTGAAGCTGAAGGACGGCGGCC
ACTACGACGCTGAGGTCAAGACCACCTACAAGGCCAAGAAGCCCG
TGCAGCTGCCCGGCGCCTACAACGTCAACATCAAGTTGGACATCA
CCTCCCACAACGAGGACTACACCATCGTGGAACAGTACGAACGCG
CCGAGGGCCGCCACTCCACCGGCGGCATGGACGAGCTGTACAAGT
AAGAATTCCTAGAGCTCGCTGATCAGCCTCGACTGTGCCTTCTAG
TTGCCAGCCATCTGTTGTTTGCCCCTCCCCCGTGCCTTCCTTGAC
CCTGGAAGGTGCCACTCCCACTGTCCTTTCCTAATAAAATGAGGA
AATTGCATCGCATTGTCTGAGTAGGTGTCATTCTATTCTGGGGGG
TGGGGTGGGGCAGGACAGCAAGGGGGAGGATTGGGAAGAGAATAG
CAGGCATGCTGGGGAGGTACCGAGGGCCTATTTCCCATGATTCCT
TCATATTTGCATATACGATACAAGGCTGTTAGAGAGATAATTAGA
ATTAATTTGACTGTAAACACAAAGATATTAGTACAAAATACGTGA
CGTAGAAAGTAATAATTTCTTGGGTAGTTTGCAGTTTTAAAATTA
TGTTTTAAAATGGACTATCATATGCTTACCGTAACTTGAAAGTAT
TTCGATTTCTTGGCTTTATATATCTTGTGGAAAGGACGAAACACC
GCTGTCTACGAAGAGCCCGTGGTTTTAGAGCTAGAAATAGCAAGT
TAAAATAAGGCTAGTCCGTTATCAACTTGAAAAAGTGGCACCGAG
TCGGTGCTTTTTTGACGCGTCCGCGTCGACATAAGAATGCGGCCG
CAGGAACCCCTAGTGATGGAGTTGGCCACTCCCTCTCTGCGCGCT
CGCTCGCTCACTGAGGCCGGGCGACCAAAGGTCGCCCGACGCCCG
GGCTTTGCCCGGGCGGCCTCAGTGAGCGAGCGAGCGCGCAGCTGC
CTGCAGGGGCGCCTGATGCGGTATTTTCTCCTTACGCATCTGTGC
GGTATTTCACACCGCATACGTCAAAGCAACCATAGTACGCGCCCT
GTAGCGGCGCATTAAGCGCGGCGGGTGTGGTGGTTACGCGCAGCG
TGACCGCTACACTTGCCAGCGCCTTAGCGCCCGCTCCTTTCGCTT
TCTTCCCTTCCTTTCTCGCCACGTTCGCCGGCTTTCCCCGTCAAG
CTCTAAATCGGGGGCTCCCTTTAGGGTTCCGATTTAGTGCTTTAC
GGCACCTCGACCCCAAAAAACTTGATTTGGGTGATGGTTCACGTA
GTGGGCCATCGCCCTGATAGACGGTTTTTCGCCCTTTGACGTTGG
AGTCCACGTTCTTTAATAGTGGACTCTTGTTCCAAACTGGAACAA
CACTCAACTCTATCTCGGGCTATTCTTTTGATTTATAAGGGATTT
TGCCGATTTCGGTCTATTGGTTAAAAAATGAGCTGATTTAACAAA
AATTTAACGCGAATTTTAACAAAATATTAACGTTTACAATTTTAT
GGTGCACTCTCAGTACAATCTGCTCTGATGCCGCATAGTTAAGCC
AGCCCCGACACCCGCCAACACCCGCTGACGCGCCCTGACGGGCTT
GTCTGCTCCCGGCATCCGCTTACAGACAAGCTGTGACCGTCTCCG
GGAGCTGCATGTGTCAGAGGTTTTCACCGTCATCACCGAAACGCG
CGAGACGAAAGGGCCTCGTGATACGCCTATTTTTATAGGTTAATG
TCATGATAATAATGGTTTCTTAGACGTCAGGTGGCACTTTTCGGG
GAAATGTGCGCGGAACCCCTATTTGTTTATTTTTCTAAATACATT
CAAATATGTATCCGCTCATGAGACAATAACCCTGATAAATGCTTC
AATAATATTGAAAAAGGAAGAGTATGAGTATTCAACATTTCCGTG
TCGCCCTTATTCCCTTTTTTGCGGCATTTTGCCTTCCTGTTTTTG
CTCACCCAGAAACGCTGGTGAAAGTAAAAGATGCTGAAGATCAGT
TGGGTGCACGAGTGGGTTACATCGAACTGGATCTCAACAGCGGTA
AGATCCTTGAGAGTTTTCGCCCCGAAGAACGTTTTCCAATGATGA
GCACTTTTAAAGTTCTGCTATGTGGCGCGGTATTATCCCGTATTG
ACGCCGGGCAAGAGCAACTCGGTCGCCGCATACACTATTCTCAGA
ATGACTTGGTTGAGTACTCACCAGTCACAGAAAAGCATCTTACGG
ATGGCATGACAGTAAGAGAATTATGCAGTGCTGCCATAACCATGA
GTGATAACACTGCGGCCAACTTACTTCTGACAACGATCGGAGGAC
CGAAGGAGCTAACCGCTTTTTTGCACAACATGGGGGATCATGTAA
CTCGCCTTGATCGTTGGGAACCGGAGCTGAATGAAGCCATACCAA
ACGACGAGCGTGACACCACGATGCCTGTAGCAATGGCAACAACGT
TGCGCAAACTATTAACTGGCGAACTACTTACTCTAGCTTCCCGGC
AACAATTAATAGACTGGATGGAGGCGGATAAAGTTGCAGGACCAC
TTCTGCGCTCGGCCCTTCCGGCTGGCTGGTTTATTGCTGATAAAT
CTGGAGCCGGTGAGCGTGGAAGCCGCGGTATCATTGCAGCACTGG
GGCCAGATGGTAAGCCCTCCCGTATCGTAGTTATCTACACGACGG
GGAGTCAGGCAACTATGGATGAACGAAATAGACAGATCGCTGAGA
TAGGTGCCTCACTGATTAAGCATTGGTAACTGTCAGACCAAGTTT
ACTCATATATACTTTAGATTGATTTAAAACTTCATTTTTAATTTA
AAAGGATCTAGGTGAAGATCCTTTTTGATAATCTCATGACCAAAA
TCCCTTAACGTGAGTTTTCGTTCCACTGAGCGTCAGACCCCGTAG
AAAAGATCAAAGGATCTTCTTGAGATCCTTTTTTTCTGCGCGTAA
TCTGCTGCTTGCAAACAAAAAAACCACCGCTACCAGCGGTGGTTT
GTTTGCCGGATCAAGAGCTACCAACTCTTTTTCCGAAGGTAACTG
GCTTCAGCAGAGCGCAGATACCAAATACTGTTCTTCTAGTGTAGC
CGTAGTTAGGCCACCACTTCAAGAACTCTGTAGCACCGCCTACAT
ACCTCGCTCTGCTAATCCTGTTACCAGTGGCTGCTGCCAGTGGCG
ATAAGTCGTGTCTTACCGGGTTGGACTCAAGACGATAGTTACCGG
ATAAGGCGCAGCGGTCGGGCTGAACGGGGGGTTCGTGCACACAGC
CCAGCTTGGAGCGAACGACCTACACCGAACTGAGATACCTACAGC
GTGAGCTATGAGAAAGCGCCACGCTTCCCGAAGGGAGAAAGGCGG
ACAGGTATCCGGTAAGCGGCAGGGTCGGAACAGGAGAGCGCACGA
GGGAGCTTCCAGGGGGAAACGCCTGGTATCTTTATAGTCCTGTCG
GGTTTCGCCACCTCTGACTTGAGCGTCGATTTTTGTGATGCTCGT
CAGGGGGGCGGAGCCTATGGAAAAACGCCAGCAACGCGGCCTTTT
TACGGTTCCTGGCCTTTTGCTGGCCTTTTGCTCACATGT
(SEQ ID NO: 32; see FIG. 24 for a plasmid map).
pAAV-hRK-mCherry-U6-gRNA1-RHO-HITI donor:
CCTGC
AGGCAGCTGCGCGCTCGCTCGCTCACTGAGGCCGCCCGGGCGT
CGGGCGACCTTTGGTCGCCCGGCCTCAGTGAGCGAGCGAGCGCGC
AGAGAGGGAGTGGCCAACTCCATCACTAGGGGTTCCTGCGGCCTT
CTAGACTCGAGTTGGGCCCCAGAAGCCTGGTGGTTGTTTGTCCTT
CTCAGGGGAAAAGTGAGGCGGCCCCTTGGAGGAAGGGGCCGGGCA
GAATGATCTAATCGGATTCCAAGCAGCTCAGGGGATTGTCTTTTT
CTAGCACCTTCTTGCCACTCCTAAGCGTCCTCCGTGACCCCGGCT
GGGATTTAGCCTGGTGCTGTGTCAGCCCCGGTCTCCCAGGGGCTT
CCCAGTGGTCCCCAGGAACCCTCGACAGGGCCCGGTCTCTCTCGT
CCAGCAAGGGCAGGGACGGGCCACAGGCCAAGGGCCCTCGATACC
GGTGCCACCATGGTGAGCAAGGGCGAGGAGGATAACATGGCCATC
ATCAAGGAGTTCATGCGCTTCAAGGTGCACATGGAGGGCTCCGTG
AACGGCCACGAGTTCGAGATCGAGGGCGAGGGCGAGGGCCGCCCC
TACGAGGGCACCCAGACCGCCAAGCTGAAGGTGACCAAGGGTGGC
CCCCTGCCCTTCGCCTGGGACATCCTGTCCCCTCAGTTCATGTAC
GGCTCCAAGGCCTACGTGAAGCACCCCGCCGACATCCCCGACTAC
TTGAAGCTGTCCTTCCCCGAGGGCTTCAAGTGGGAGCGCGTGATG
AACTTCGAGGACGGCGGCGTGGTGACCGTGACCCAGGACTCCTCC
CTGCAGGACGGCGAGTTCATCTACAAGGTGAAGCTGCGCGGCACC
AACTTCCCCTCCGACGGCCCCGTAATGCAGAAGAAGACCATGGGC
TGGGAGGCCTCCTCCGAGCGGATGTACCCCGAGGACGGCGCCCTG
AAGGGCGAGATCAAGCAGAGGCTGAAGCTGAAGGACGGCGGCCAC
TACGACGCTGAGGTCAAGACCACCTACAAGGCCAAGAAGCCCGTG
CAGCTGCCCGGCGCCTACAACGTCAACATCAAGTTGGACATCACC
TCCCACAACGAGGACTACACCATCGTGGAACAGTACGAACGCGCC
GAGGGCCGCCACTCCACCGGCGGCATGGACGAGCTGTACAAGTAA
GAATTCCTAGAGCTCGCTGATCAGCCTCGACTGTGCCTTCTAGTT
GCCAGCCATCTGTTGTTTGCCCCTCCCCCGTGCCTTCCTTGACCC
TGGAAGGTGCCACTCCCACTGTCCTTTCCTAATAAAATGAGGAAA
TTGCATCGCATTGTCTGAGTAGGTGTCATTCTATTCTGGGGGGTG
GGGTGGGGCAGGACAGCAAGGGGGAGGATTGGGAAGAGAATAGCA
GGCATGCTGGGGAGGTACCGAGGGCCTATTTCCCATGATTCCTTC
ATATTTGCATATACGATACAAGGCTGTTAGAGAGATAATTAGAAT
TAATTTGACTGTAAACACAAAGATATTAGTACAAAATACGTGACG
TAGAAAGTAATAATTTCTTGGGTAGTTTGCAGTTTTAAAATTATG
TTTTAAAATGGACTATCATATGCTTACCGTAACTTGAAAGTATTT
CGATTTCTTGGCTTTATATATCTTGTGGAAAGGACGAAACACCGC
TGTCTACGAAGAGCCCGTGGTTTTAGAGCTAGAAATAGCAAGTTA
AAATAAGGCTAGTCCGTTATCAACTTGAAAAAGTGGCACCGAGTC
GGTGCTTTTTTGACGCGTCCCCACGGGCTCTTCGTAGACAGAGCC
GCAGCCATGAACGGCACAGAGGGCCCCAATTTTTATGTGCCCTTC
TCCAACGTCACAGGCGTGGTGCGGAGCCCCTTCGAGCAGCCGCAG
TACTACCTGGCGGAACCATGGCAGTTCTCCATGCTGGCAGCGTAC
ATGTTCCTGCTCATCGTGCTGGGCTTCCCCATCAACTTCCTCACG
CTCTACGTCACCGTACAGCACAAGAAGCTGCGCACACCCCTCAAC
TACATCCTGCTCAACTTGGCCGTGGCTGACCTCTTCATGGTCTTC
GGAGGATTCACCACCACCCTCTACACATCACTCCATGGCTACTTC
GTCTTTGGGCCCACAGGCTGTAATCTCGAGGGCTTCTTTGCCACA
CTTGGAGGTGAAATCGCCCTGTGGTCCCTGGTGGTCCTGGCCATT
GAGCGCTACGTGGTGGTCTGCAAGCCGATGAGCAACTTCCGCTTC
GGGGAGAATCACGCTATCATGGGTGTGGTCTTCACCTGGATCATG
GCGTTGGCCTGTGCTGCTCCCCCACTCGTTGGCTGGTCCAGGTAC
ATCCCTGAGGGCATGCAATGTTCATGCGGGATTGACTACTACACA
CTCAAGCCTGAGGTCAACAACGAATCCTTTGTCATCTACATGTTC
GTGGTCCACTTCACCATTCCTATGATCGTCATCTTCTTCTGCTAT
GGGCAGCTGGTCTTCACAGTCAAGGAGGCGGCTGCCCAGCAGCAG
GAGTCAGCCACCACTCAGAAGGCAGAGAAGGAAGTCACCCGCATG
GTTATCATCATGGTCATCTTCTTCCTGATCTGCTGGCTTCCCTAC
GCCAGTGTGGCCTTCTACATCTTCACCCACCAGGGCTCCAACTTC
GGCCCCATCTTCATGACTCTGCCAGCTTTCTTTGCTAAGAGCTCT
TCCATCTATAACCCGGTCATCTACATCATGTTGAACAAGCAGTTC
CGGAACTGTATGCTCACCACGCTGTGCTGCGGCAAGAATCCACTG
GGAGATGACGACGCCTCTGCCACCGCTTCCAAGACGGAGACCAGC
CAGGTGGCTCCAGCCTAATAAGTCGACCCCCACGGGCTCTTCGTA
GACAGGCGGCCGCAGGAACCCCTAGTGATGGAGTTGGCCACTCCC
TCTCTGCGCGCTCGCTCGCTCACTGAGGCCGGGCGACCAAAGGTC
GCCCGACGCCCGGGCTTTGCCCGGGCGGCCTCAGTGAGCGAGCGA
GCGCGCAGCTGCCTGCAGGGGCGCCTGATGCGGTATTTTCTCCTT
ACGCATCTGTGCGGTATTTCACACCGCATACGTCAAAGCAACCAT
AGTACGCGCCCTGTAGCGGCGCATTAAGCGCGGCGGGTGTGGTGG
TTACGCGCAGCGTGACCGCTACACTTGCCAGCGCCTTAGCGCCCG
CTCCTTTCGCTTTCTTCCCTTCCTTTCTCGCCACGTTCGCCGGCT
TTCCCCGTCAAGCTCTAAATCGGGGGCTCCCTTTAGGGTTCCGAT
TTAGTGCTTTACGGCACCTCGACCCCAAAAAACTTGATTTGGGTG
ATGGTTCACGTAGTGGGCCATCGCCCTGATAGACGGTTTTTCGCC
CTTTGACGTTGGAGTCCACGTTCTTTAATAGTGGACTCTTGTTCC
AAACTGGAACAACACTCAACTCTATCTCGGGCTATTCTTTTGATT
TATAAGGGATTTTGCCGATTTCGGTCTATTGGTTAAAAAATGAGC
TGATTTAACAAAAATTTAACGCGAATTTTAACAAAATATTAACGT
TTACAATTTTATGGTGCACTCTCAGTACAATCTGCTCTGATGCCG
CATAGTTAAGCCAGCCCCGACACCCGCCAACACCCGCTGACGCGC
CCTGACGGGCTTGTCTGCTCCCGGCATCCGCTTACAGACAAGCTG
TGACCGTCTCCGGGAGCTGCATGTGTCAGAGGTTTTCACCGTCAT
CACCGAAACGCGCGAGACGAAAGGGCCTCGTGATACGCCTATTTT
TATAGGTTAATGTCATGATAATAATGGTTTCTTAGACGTCAGGTG
GCACTTTTCGGGGAAATGTGCGCGGAACCCCTATTTGTTTATTTT
TCTAAATACATTCAAATATGTATCCGCTCATGAGACAATAACCCT
GATAAATGCTTCAATAATATTGAAAAAGGAAGAGTATGAGTATTC
AACATTTCCGTGTCGCCCTTATTCCCTTTTTTGCGGCATTTTGCC
TTCCTGTTTTTGCTCACCCAGAAACGCTGGTGAAAGTAAAAGATG
CTGAAGATCAGTTGGGTGCACGAGTGGGTTACATCGAACTGGATC
TCAACAGCGGTAAGATCCTTGAGAGTTTTCGCCCCGAAGAACGTT
TTCCAATGATGAGCACTTTTAAAGTTCTGCTATGTGGCGCGGTAT
TATCCCGTATTGACGCCGGGCAAGAGCAACTCGGTCGCCGCATAC
ACTATTCTCAGAATGACTTGGTTGAGTACTCACCAGTCACAGAAA
AGCATCTTACGGATGGCATGACAGTAAGAGAATTATGCAGTGCTG
CCATAACCATGAGTGATAACACTGCGGCCAACTTACTTCTGACAA
CGATCGGAGGACCGAAGGAGCTAACCGCTTTTTTGCACAACATGG
GGGATCATGTAACTCGCCTTGATCGTTGGGAACCGGAGCTGAATG
AAGCCATACCAAACGACGAGCGTGACACCACGATGCCTGTAGCAA
TGGCAACAACGTTGCGCAAACTATTAACTGGCGAACTACTTACTC
TAGCTTCCCGGCAACAATTAATAGACTGGATGGAGGCGGATAAAG
TTGCAGGACCACTTCTGCGCTCGGCCCTTCCGGCTGGCTGGTTTA
TTGCTGATAAATCTGGAGCCGGTGAGCGTGGAAGCCGCGGTATCA
TTGCAGCACTGGGGCCAGATGGTAAGCCCTCCCGTATCGTAGTTA
TCTACACGACGGGGAGTCAGGCAACTATGGATGAACGAAATAGAC
AGATCGCTGAGATAGGTGCCTCACTGATTAAGCATTGGTAACTGT
CAGACCAAGTTTACTCATATATACTTTAGATTGATTTAAAACTTC
ATTTTTAATTTAAAAGGATCTAGGTGAAGATCCTTTTTGATAATC
TCATGACCAAAATCCCTTAACGTGAGTTTTCGTTCCACTGAGCGT
CAGACCCCGTAGAAAAGATCAAAGGATCTTCTTGAGATCCTTTTT
TTCTGCGCGTAATCTGCTGCTTGCAAACAAAAAAACCACCGCTAC
CAGCGGTGGTTTGTTTGCCGGATCAAGAGCTACCAACTCTTTTTC
CGAAGGTAACTGGCTTCAGCAGAGCGCAGATACCAAATACTGTTC
TTCTAGTGTAGCCGTAGTTAGGCCACCACTTCAAGAACTCTGTAG
CACCGCCTACATACCTCGCTCTGCTAATCCTGTTACCAGTGGCTG
CTGCCAGTGGCGATAAGTCGTGTCTTACCGGGTTGGACTCAAGAC
GATAGTTACCGGATAAGGCGCAGCGGTCGGGCTGAACGGGGGGTT
CGTGCACACAGCCCAGCTTGGAGCGAACGACCTACACCGAACTGA
GATACCTACAGCGTGAGCTATGAGAAAGCGCCACGCTTCCCGAAG
GGAGAAAGGCGGACAGGTATCCGGTAAGCGGCAGGGTCGGAACAG
GAGAGCGCACGAGGGAGCTTCCAGGGGGAAACGCCTGGTATCTTT
ATAGTCCTGTCGGGTTTCGCCACCTCTGACTTGAGCGTCGATTTT
TGTGATGCTCGTCAGGGGGGCGGAGCCTATGGAAAAACGCCAGCA
ACGCGGCCTTTTTACGGTTCCTGGCCTTTTGCTGGCCTTTTGCTC
ACATGT
(SEQ ID NO: 33; see FIG. 25 for a plasmid map).
pAAV-hRK-mCherry-U6-gRNA1-GFP-HITI donor:
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CTCTAGACTCGAGTTGGGCCCCAGAAGCCTGGTGGTTGTTTGTCC
TTCTCAGGGGAAAAGTGAGGCGGCCCCTTGGAGGAAGGGGCCGGG
CAGAATGATCTAATCGGATTCCAAGCAGCTCAGGGGATTGTCTTT
TTCTAGCACCTTCTTGCCACTCCTAAGCGTCCTCCGTGACCCCGG
CTGGGATTTAGCCTGGTGCTGTGTCAGCCCCGGTCTCCCAGGGGC
TTCCCAGTGGTCCCCAGGAACCCTCGACAGGGCCCGGTCTCTCTC
GTCCAGCAAGGGCAGGGACGGGCCACAGGCCAAGGGCCCTCGATA
CCGGTGCCACCATGGTGAGCAAGGGCGAGGAGGATAACATGGCCA
TCATCAAGGAGTTCATGCGCTTCAAGGTGCACATGGAGGGCTCCG
TGAACGGCCACGAGTTCGAGATCGAGGGCGAGGGCGAGGGCCGCC
CCTACGAGGGCACCCAGACCGCCAAGCTGAAGGTGACCAAGGGTG
GCCCCCTGCCCTTCGCCTGGGACATCCTGTCCCCTCAGTTCATGT
ACGGCTCCAAGGCCTACGTGAAGCACCCCGCCGACATCCCCGACT
ACTTGAAGCTGTCCTTCCCCGAGGGCTTCAAGTGGGAGCGCGTGA
TGAACTTCGAGGACGGCGGCGTGGTGACCGTGACCCAGGACTCCT
CCCTGCAGGACGGCGAGTTCATCTACAAGGTGAAGCTGCGCGGCA
CCAACTTCCCCTCCGACGGCCCCGTAATGCAGAAGAAGACCATGG
GCTGGGAGGCCTCCTCCGAGCGGATGTACCCCGAGGACGGCGCCC
TGAAGGGCGAGATCAAGCAGAGGCTGAAGCTGAAGGACGGCGGCC
ACTACGACGCTGAGGTCAAGACCACCTACAAGGCCAAGAAGCCCG
TGCAGCTGCCCGGCGCCTACAACGTCAACATCAAGTTGGACATCA
CCTCCCACAACGAGGACTACACCATCGTGGAACAGTACGAACGCG
CCGAGGGCCGCCACTCCACCGGCGGCATGGACGAGCTGTACAAGT
AAGAATTCCTAGAGCTCGCTGATCAGCCTCGACTGTGCCTTCTAG
TTGCCAGCCATCTGTTGTTTGCCCCTCCCCCGTGCCTTCCTTGAC
CCTGGAAGGTGCCACTCCCACTGTCCTTTCCTAATAAAATGAGGA
AATTGCATCGCATTGTCTGAGTAGGTGTCATTCTATTCTGGGGGG
TGGGGTGGGGCAGGACAGCAAGGGGGAGGATTGGGAAGAGAATAG
CAGGCATGCTGGGGAGGTACCGAGGGCCTATTTCCCATGATTCCT
TCATATTTGCATATACGATACAAGGCTGTTAGAGAGATAATTAGA
ATTAATTTGACTGTAAACACAAAGATATTAGTACAAAATACGTGA
CGTAGAAAGTAATAATTTCTTGGGTAGTTTGCAGTTTTAAAATTA
TGTTTTAAAATGGACTATCATATGCTTACCGTAACTTGAAAGTAT
TTCGATTTCTTGGCTTTATATATCTTGTGGAAAGGACGAAACACC
GCTGTCTACGAAGAGCCCGTGGTTTTAGAGCTAGAAATAGCAAGT
TAAAATAAGGCTAGTCCGTTATCAACTTGAAAAAGTGGCACCGAG
TCGGTGCTTTTTTGACGCGTCCCCACGGGCTCTTCGTAGACAGAG
CCGCAGCCATGGTGAGCAAGGGCGAGGAGCTGTTCACCGGGGTGG
TGCCCATCCTGGTCGAGCTGGACGGCGACGTAAACGGCCACAAGT
TCAGCGTGTCCGGCGAGGGCGAGGGCGATGCCACCTACGGCAAGC
TGACCCTGAAGTTCATCTGCACCACCGGCAAGCTGCCCGTGCCCT
GGCCCACCCTCGTGACCACCCTGACCTACGGCGTGCAGTGCTTCA
GCCGCTACCCCGACCACATGAAGCAGCACGACTTCTTCAAGTCCG
CCATGCCCGAAGGCTACGTCCAGGAGCGCACCATCTTCTTCAAGG
ACGACGGCAACTACAAGACCCGCGCCGAGGTGAAGTTCGAGGGCG
ACACCCTGGTGAACCGCATCGAGCTGAAGGGCATCGACTTCAAGG
AGGACGGCAACATCCTGGGGCACAAGCTGGAGTACAACTACAACA
GCCACAACGTCTATATCATGGCCGACAAGCAGAAGAACGGCATCA
AGGTGAACTTCAAGATCCGCCACAACATCGAGGACGGCAGCGTGC
AGCTCGCCGACCACTACCAGCAGAACACCCCCATCGGCGACGGCC
CCGTGCTGCTGCCCGACAACCACTACCTGAGCACCCAGTCCGCCC
TGAGCAAAGACCCCAACGAGAAGCGCGATCACATGGTCCTGCTGG
AGTTCGTGACCGCCGCCGGGATCACTCTCGGCATGGACGAGCTGT
ACAAGTAATAAGTCGACCCCCACGGGCTCTTCGTAGACAGGCGGC
CGCAGGAACCCCTAGTGATGGAGTTGGCCACTCCCTCTCTGCGCG
CTCGCTCGCTCACTGAGGCCGGGCGACCAAAGGTCGCCCGACGCC
CGGGCTTTGCCCGGGCGGCCTCAGTGAGCGAGCGAGCGCGCAGCT
GCCTGCAGGGGCGCCTGATGCGGTATTTTCTCCTTACGCATCTGT
GCGGTATTTCACACCGCATACGTCAAAGCAACCATAGTACGCGCC
CTGTAGCGGCGCATTAAGCGCGGCGGGTGTGGTGGTTACGCGCAG
CGTGACCGCTACACTTGCCAGCGCCTTAGCGCCCGCTCCTTTCGC
TTTCTTCCCTTCCTTTCTCGCCACGTTCGCCGGCTTTCCCCGTCA
AGCTCTAAATCGGGGGCTCCCTTTAGGGTTCCGATTTAGTGCTTT
ACGGCACCTCGACCCCAAAAAACTTGATTTGGGTGATGGTTCACG
TAGTGGGCCATCGCCCTGATAGACGGTTTTTCGCCCTTTGACGTT
GGAGTCCACGTTCTTTAATAGTGGACTCTTGTTCCAAACTGGAAC
AACACTCAACTCTATCTCGGGCTATTCTTTTGATTTATAAGGGAT
TTTGCCGATTTCGGTCTATTGGTTAAAAAATGAGCTGATTTAACA
AAAATTTAACGCGAATTTTAACAAAATATTAACGTTTACAATTTT
ATGGTGCACTCTCAGTACAATCTGCTCTGATGCCGCATAGTTAAG
CCAGCCCCGACACCCGCCAACACCCGCTGACGCGCCCTGACGGGC
TTGTCTGCTCCCGGCATCCGCTTACAGACAAGCTGTGACCGTCTC
CGGGAGCTGCATGTGTCAGAGGTTTTCACCGTCATCACCGAAACG
CGCGAGACGAAAGGGCCTCGTGATACGCCTATTTTTATAGGTTAA
TGTCATGATAATAATGGTTTCTTAGACGTCAGGTGGCACTTTTCG
GGGAAATGTGCGCGGAACCCCTATTTGTTTATTTTTCTAAATACA
TTCAAATATGTATCCGCTCATGAGACAATAACCCTGATAAATGCT
TCAATAATATTGAAAAAGGAAGAGTATGAGTATTCAACATTTCCG
TGTCGCCCTTATTCCCTTTTTTGCGGCATTTTGCCTTCCTGTTTT
TGCTCACCCAGAAACGCTGGTGAAAGTAAAAGATGCTGAAGATCA
GTTGGGTGCACGAGTGGGTTACATCGAACTGGATCTCAACAGCGG
TAAGATCCTTGAGAGTTTTCGCCCCGAAGAACGTTTTCCAATGAT
GAGCACTTTTAAAGTTCTGCTATGTGGCGCGGTATTATCCCGTAT
TGACGCCGGGCAAGAGCAACTCGGTCGCCGCATACACTATTCTCA
GAATGACTTGGTTGAGTACTCACCAGTCACAGAAAAGCATCTTAC
GGATGGCATGACAGTAAGAGAATTATGCAGTGCTGCCATAACCAT
GAGTGATAACACTGCGGCCAACTTACTTCTGACAACGATCGGAGG
ACCGAAGGAGCTAACCGCTTTTTTGCACAACATGGGGGATCATGT
AACTCGCCTTGATCGTTGGGAACCGGAGCTGAATGAAGCCATACC
AAACGACGAGCGTGACACCACGATGCCTGTAGCAATGGCAACAAC
GTTGCGCAAACTATTAACTGGCGAACTACTTACTCTAGCTTCCCG
GCAACAATTAATAGACTGGATGGAGGCGGATAAAGTTGCAGGACC
ACTTCTGCGCTCGGCCCTTCCGGCTGGCTGGTTTATTGCTGATAA
ATCTGGAGCCGGTGAGCGTGGAAGCCGCGGTATCATTGCAGCACT
GGGGCCAGATGGTAAGCCCTCCCGTATCGTAGTTATCTACACGAC
GGGGAGTCAGGCAACTATGGATGAACGAAATAGACAGATCGCTGA
GATAGGTGCCTCACTGATTAAGCATTGGTAACTGTCAGACCAAGT
TTACTCATATATACTTTAGATTGATTTAAAACTTCATTTTTAATT
TAAAAGGATCTAGGTGAAGATCCTTTTTGATAATCTCATGACCAA
AATCCCTTAACGTGAGTTTTCGTTCCACTGAGCGTCAGACCCCGT
AGAAAAGATCAAAGGATCTTCTTGAGATCCTTTTTTTCTGCGCGT
AATCTGCTGCTTGCAAACAAAAAAACCACCGCTACCAGCGGTGGT
TTGTTTGCCGGATCAAGAGCTACCAACTCTTTTTCCGAAGGTAAC
TGGCTTCAGCAGAGCGCAGATACCAAATACTGTTCTTCTAGTGTA
GCCGTAGTTAGGCCACCACTTCAAGAACTCTGTAGCACCGCCTAC
ATACCTCGCTCTGCTAATCCTGTTACCAGTGGCTGCTGCCAGTGG
CGATAAGTCGTGTCTTACCGGGTTGGACTCAAGACGATAGTTACC
GGATAAGGCGCAGCGGTCGGGCTGAACGGGGGGTTCGTGCACACA
GCCCAGCTTGGAGCGAACGACCTACACCGAACTGAGATACCTACA
GCGTGAGCTATGAGAAAGCGCCACGCTTCCCGAAGGGAGAAAGGC
GGACAGGTATCCGGTAAGCGGCAGGGTCGGAACAGGAGAGCGCAC
GAGGGAGCTTCCAGGGGGAAACGCCTGGTATCTTTATAGTCCTGT
CGGGTTTCGCCACCTCTGACTTGAGCGTCGATTTTTGTGATGCTC
GTCAGGGGGGCGGAGCCTATGGAAAAACGCCAGCAACGCGGCCTT
TTTACGGTTCCTGGCCTTTTGCTGGCCTTTTGCTCACATGT
(SEQ ID NO: 34; see FIG. 26 for a plasmid map).

The dual Kozak reporter plasmids containing the fragment of HITI donor integrated into RHO locus, pCMV-Kozak-GFP-Stop-Kozak-RHO-Stop (SEQ ID NO:35) and pCMV-Kozak-RHO-Stop-Kozak-GFP-Stop (SEQ ID NO:36), were constructed. To construct CMV-Kozak-GFP-Stop-Kozak-RHO-Stop, the sequence containing a Kozak motif between the integrated gene and the disrupted endogenous RHO (Kozak-RHO-Stop) was amplified and inserted into pCMV backbone plasmid (pAAV-CMV-SpCas9-U6-Bsal-gRNA) via restricted enzyme sties (SalI and MluI) to create pCMV-Kozak-RHO-Stop. Next, the Kozak-GFP-Stop fragment, mimicking the integrated fragment of 5′ UTR RHO induced by SpCas9-RHO KI, was amplified and sub-cloned into pCMV-Kozak-RHO-Stop to generate the pCMV-Kozak-GFP-Stop-Kozak-RHO-Stop. A similar approach was used to construct pCMV-Kozak-RHO-Stop-Kozak-GFP-Stop.

The nucleotide sequences of reporter plasmids are provided as follows:

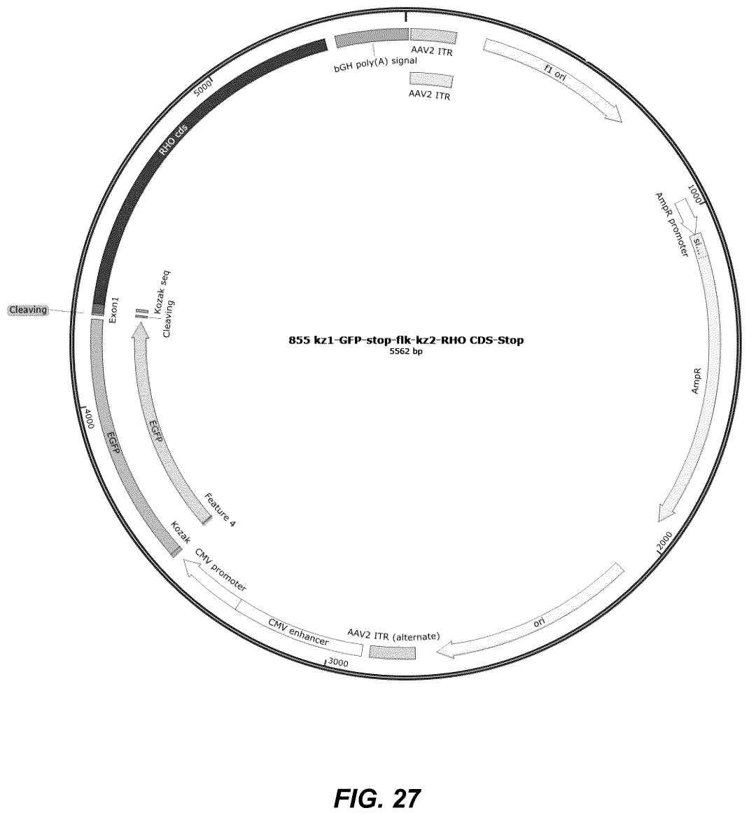

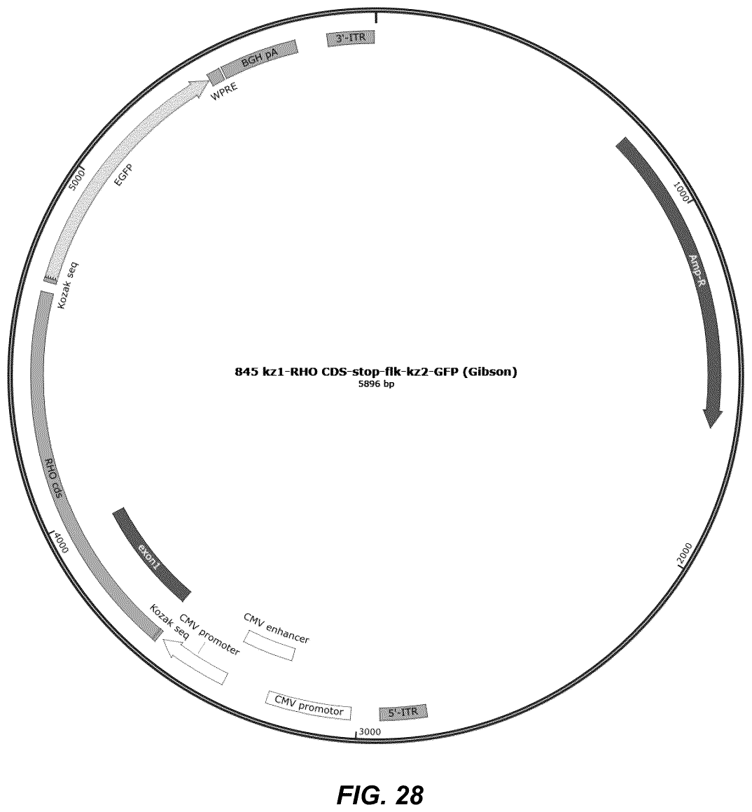

Kozak-GFP-Kozak-RHO:
GCTGGGGAGGGCCGCAGGAACCCCTAGTGATGGAGTTGGCCACTC
CCTCTCTGCGCGCTCGCTCGCTCACTGAGGCCGGGCGACCAAAGG
TCGCCCGACGCCCGGGCTTTGCCCGGGCGGCCTCAGTGAGCGAGC
GAGCGCGCAGCTGCCTGCAGGGGCGCCTGATGCGGTATTTTCTCC
TTACGCATCTGTGCGGTATTTCACACCGCATACGTCAAAGCAACC
ATAGTACGCGCCCTGTAGCGGCGCATTAAGCGCGGCGGGTGTGGT
GGTTACGCGCAGCGTGACCGCTACACTTGCCAGCGCCTTAGCGCC
CGCTCCTTTCGCTTTCTTCCCTTCCTTTCTCGCCACGTTCGCCGG
CTTTCCCCGTCAAGCTCTAAATCGGGGGCTCCCTTTAGGGTTCCG
ATTTAGTGCTTTACGGCACCTCGACCCCAAAAAACTTGATTTGGG
TGATGGTTCACGTAGTGGGCCATCGCCCTGATAGACGGTTTTTCG
CCCTTTGACGTTGGAGTCCACGTTCTTTAATAGTGGACTCTTGTT
CCAAACTGGAACAACACTCAACTCTATCTCGGGCTATTCTTTTGA
TTTATAAGGGATTTTGCCGATTTCGGTCTATTGGTTAAAAAATGA
GCTGATTTAACAAAAATTTAACGCGAATTTTAACAAAATATTAAC
GTTTACAATTTTATGGTGCACTCTCAGTACAATCTGCTCTGATGC
CGCATAGTTAAGCCAGCCCCGACACCCGCCAACACCCGCTGACGC
GCCCTGACGGGCTTGTCTGCTCCCGGCATCCGCTTACAGACAAGC
TGTGACCGTCTCCGGGAGCTGCATGTGTCAGAGGTTTTCACCGTC
ATCACCGAAACGCGCGAGACGAAAGGGCCTCGTGATACGCCTATT
TTTATAGGTTAATGTCATGATAATAATGGTTTCTTAGACGTCAGG
TGGCACTTTTCGGGGAAATGTGCGCGGAACCCCTATTTGTTTATT
TTTCTAAATACATTCAAATATGTATCCGCTCATGAGACAATAACC
CTGATAAATGCTTCAATAATATTGAAAAAGGAAGAGTATGAGTAT
TCAACATTTCCGTGTCGCCCTTATTCCCTTTTTTGCGGCATTTTG
CCTTCCTGTTTTTGCTCACCCAGAAACGCTGGTGAAAGTAAAAGA
TGCTGAAGATCAGTTGGGTGCACGAGTGGGTTACATCGAACTGGA
TCTCAACAGCGGTAAGATCCTTGAGAGTTTTCGCCCCGAAGAACG
TTTTCCAATGATGAGCACTTTTAAAGTTCTGCTATGTGGCGCGGT
ATTATCCCGTATTGACGCCGGGCAAGAGCAACTCGGTCGCCGCAT
ACACTATTCTCAGAATGACTTGGTTGAGTACTCACCAGTCACAGA
AAAGCATCTTACGGATGGCATGACAGTAAGAGAATTATGCAGTGC
TGCCATAACCATGAGTGATAACACTGCGGCCAACTTACTTCTGAC
AACGATCGGAGGACCGAAGGAGCTAACCGCTTTTTTGCACAACAT
GGGGGATCATGTAACTCGCCTTGATCGTTGGGAACCGGAGCTGAA
TGAAGCCATACCAAACGACGAGCGTGACACCACGATGCCTGTAGC
AATGGCAACAACGTTGCGCAAACTATTAACTGGCGAACTACTTAC
TCTAGCTTCCCGGCAACAATTAATAGACTGGATGGAGGCGGATAA
AGTTGCAGGACCACTTCTGCGCTCGGCCCTTCCGGCTGGCTGGTT
TATTGCTGATAAATCTGGAGCCGGTGAGCGTGGAAGCCGCGGTAT
CATTGCAGCACTGGGGCCAGATGGTAAGCCCTCCCGTATCGTAGT
TATCTACACGACGGGGAGTCAGGCAACTATGGATGAACGAAATAG
ACAGATCGCTGAGATAGGTGCCTCACTGATTAAGCATTGGTAACT
GTCAGACCAAGTTTACTCATATATACTTTAGATTGATTTAAAACT
TCATTTTTAATTTAAAAGGATCTAGGTGAAGATCCTTTTTGATAA
TCTCATGACCAAAATCCCTTAACGTGAGTTTTCGTTCCACTGAGC
GTCAGACCCCGTAGAAAAGATCAAAGGATCTTCTTGAGATCCTTT
TTTTCTGCGCGTAATCTGCTGCTTGCAAACAAAAAAACCACCGCT
ACCAGCGGTGGTTTGTTTGCCGGATCAAGAGCTACCAACTCTTTT
TCCGAAGGTAACTGGCTTCAGCAGAGCGCAGATACCAAATACTGT
TCTTCTAGTGTAGCCGTAGTTAGGCCACCACTTCAAGAACTCTGT
AGCACCGCCTACATACCTCGCTCTGCTAATCCTGTTACCAGTGGC
TGCTGCCAGTGGCGATAAGTCGTGTCTTACCGGGTTGGACTCAAG
ACGATAGTTACCGGATAAGGCGCAGCGGTCGGGCTGAACGGGGGG
TTCGTGCACACAGCCCAGCTTGGAGCGAACGACCTACACCGAACT
GAGATACCTACAGCGTGAGCTATGAGAAAGCGCCACGCTTCCCGA
AGGGAGAAAGGCGGACAGGTATCCGGTAAGCGGCAGGGTCGGAAC
AGGAGAGCGCACGAGGGAGCTTCCAGGGGGAAACGCCTGGTATCT
TTATAGTCCTGTCGGGTTTCGCCACCTCTGACTTGAGCGTCGATT
TTTGTGATGCTCGTCAGGGGGGCGGAGCCTATGGAAAAACGCCAG
CAACGCGGCCTTTTTACGGTTCCTGGCCTTTTGCTGGCCTTTTGC
TCACATGTCCTGCAGGCAGCTGCGCGCTCGCTCGCTCACTGAGGC
CGCCCGGGCGTCGGGCGACCTTTGGTCGCCCGGCCTCAGTGAGCG
AGCGAGCGCGCAGAGAGGGAGTGGCCAACTCCATCACTAGGGGTT
CCTGCGGCCTCTAGACTCGAGGCGTTGACATTGATTATTGACTAG
TTATTAATAGTAATCAATTACGGGGTCATTAGTTCATAGCCCATA
TATGGAGTTCCGCGTTACATAACTTACGGTAAATGGCCCGCCTGG
CTGACCGCCCAACGACCCCCGCCCATTGACGTCAATAATGACGTA
TGTTCCCATAGTAACGCCAATAGGGACTTTCCATTGACGTCAATG
GGTGGAGTATTTACGGTAAACTGCCCACTTGGCAGTACATCAAGT
GTATCATATGCCAAGTACGCCCCCTATTGACGTCAATGACGGTAA
ATGGCCCGCCTGGCATTATGCCCAGTACATGACCTTATGGGACTT
TCCTACTTGGCAGTACATCTACGTATTAGTCATCGCTATTACCAT
GGTGATGCGGTTTTGGCAGTACATCAATGGGCGTGGATAGCGGTT
TGACTCACGGGGATTTCCAAGTCTCCACCCCATTGACGTCAATGG
GAGTTTGTTTTGGCACCAAAATCAACGGGACTTTCCAAAATGTCG
TAACAACTCCGCCCCATTGACGCAAATGGGCGGTAGGCGTGTACG
GTGGGAGGTCTATATAAGCAGAGCTCTCTGGCTAACTACCGGTAG
ACAGAGCCGCAGCCATGGTGAGCAAGGGCGAGGAGCTGTTCACCG
GGGTGGTGCCCATCCTGGTCGAGCTGGACGGCGACGTAAACGGCC
ACAAGTTCAGCGTGTCCGGCGAGGGCGAGGGCGATGCCACCTACG
GCAAGCTGACCCTGAAGTTCATCTGCACCACCGGCAAGCTGCCCG
TGCCCTGGCCCACCCTCGTGACCACCCTGACCTACGGCGTGCAGT
GCTTCAGCCGCTACCCCGACCACATGAAGCAGCACGACTTCTTCA
AGTCCGCCATGCCCGAAGGCTACGTCCAGGAGCGCACCATCTTCT
TCAAGGACGACGGCAACTACAAGACCCGCGCCGAGGTGAAGTTCG
AGGGCGACACCCTGGTGAACCGCATCGAGCTGAAGGGCATCGACT
TCAAGGAGGACGGCAACATCCTGGGGCACAAGCTGGAGTACAACT
ACAACAGCCACAACGTCTATATCATGGCCGACAAGCAGAAGAACG
GCATCAAGGTGAACTTCAAGATCCGCCACAACATCGAGGACGGCA
GCGTGCAGCTCGCCGACCACTACCAGCAGAACACCCCCATCGGCG
ACGGCCCCGTGCTGCTGCCCGACAACCACTACCTGAGCACCCAGT
CCGCCCTGAGCAAAGACCCCAACGAGAAGCGCGATCACATGGTCC
TGCTGGAGTTCGTGACCGCCGCCGGGATCACTCTCGGCATGGACG
AGCTGTACAAGTAATAAGTCGACCCCCACGTGGGGCAGCCTCGAG
AGCCGCAGCCATGAACGGCACAGAGGGCCCCAATTTTTATGTGCC
CTTCTCCAACGTCACAGGCGTGGTGCGGAGCCCCTTCGAGCAGCC
GCAGTACTACCTGGCGGAACCATGGCAGTTCTCCATGCTGGCAGC
GTACATGTTCCTGCTCATCGTGCTGGGCTTCCCCATCAACTTCCT
CACGCTCTACGTCACCGTACAGCACAAGAAGCTGCGCACACCCCT
CAACTACATCCTGCTCAACTTGGCCGTGGCTGACCTCTTCATGGT
CTTCGGAGGATTCACCACCACCCTCTACACATCACTCCATGGCTA
CTTCGTCTTTGGGCCCACAGGCTGTAATCTCGAGGGCTTCTTTGC
CACACTTGGAGGTGAAATCGCCCTGTGGTCCCTGGTGGTCCTGGC
CATTGAGCGCTACGTGGTGGTCTGCAAGCCGATGAGCAACTTCCG
CTTCGGGGAGAATCACGCTATCATGGGTGTGGTCTTCACCTGGAT
CATGGCGTTGGCCTGTGCTGCTCCCCCACTCGTTGGCTGGTCCAG
GTACATCCCTGAGGGCATGCAATGTTCATGCGGGATTGACTACTA
CACACTCAAGCCTGAGGTCAACAACGAATCCTTTGTCATCTACAT
GTTCGTGGTCCACTTCACCATTCCTATGATCGTCATCTTCTTCTG
CTATGGGCAGCTGGTCTTCACAGTCAAGGAGGCGGCTGCCCAGCA
GCAGGAGTCAGCCACCACTCAGAAGGCAGAGAAGGAAGTCACCCG
CATGGTTATCATCATGGTCATCTTCTTCCTGATCTGCTGGCTTCC
CTACGCCAGTGTGGCCTTCTACATCTTCACCCACCAGGGCTCCAA
CTTCGGCCCCATCTTCATGACTCTGCCAGCTTTCTTTGCTAAGAG
CTCTTCCATCTATAACCCGGTCATCTACATCATGTTGAACAAGCA
GTTCCGGAACTGTATGCTCACCACGCTGTGCTGCGGCAAGAATCC
ACTGGGAGATGACGACGCCTCTGCCACCGCTTCCAAGACGGAGAC
CAGCCAGGTGGCTCCAGCCTAAGAATTCCTAGAGCTCGCTGATCA
GCCTCGACTGTGCCTTCTAGTTGCCAGCCATCTGTTGTTTGCCCC
TCCCCCGTGCCTTCCTTGACCCTGGAAGGTGCCACTCCCACTGTC
CTTTCCTAATAAAATGAGGAAATTGCATCGCATTGTCTGAGTAGG
TGTCATTCTATTCTGGGGGGTGGGGTGGGGCAGGACAGCAAGGGG
GAGGATTGGGAAGAGAATAGCAGGCAT
(SEQ ID NO: 35; see FIG.27 for a plasmid map).
Kozak-RHO-Kozak-GFP:
AATTAACCTAATTCACTGGCCGTCGTTTTACAACGTCGTGACTGG
GAAAACCCTGGCGTTACCCAACTTAATCGCCTTGCAGCACATCCC
CCTTTCGCCAGCTGGCGTAATAGCGAAGAGGCCCGCACCGATCGC
CCTTCCCAACAGTTGCGCAGCCTGAATGGCGAATGGGACGCGCCC
TGTAGCGGCGCATTAAGCGCGGCGGGTGTGGTGGTTACGCGCAGC
GTGACCGCTACACTTGCCAGCGCCCTAGCGCCCGCTCCTTTCGCT
TTCTTCCCTTCCTTTCTCGCCACGTTCGCCGGCTTTCCCCGTCAA
GCTCTAAATCGGGGGCTCCCTTTAGGGTTCCGATTTAGTGCTTTA
CGGCACCTCGACCCCAAAAAACTTGATTAGGGTGATGGTTCACGT
AGTGGGCCATCGCCCTGATAGACGGTTTTTCGCCCTTTGACGTTG
GAGTCCACGTTCTTTAATAGTGGACTCTTGTTCCAAACTGGAACA
ACACTCAACCCTATCTCGGTCTATTCTTTTGATTTATAAGGGATT
TTGCCGATTTCGGCCTATTGGTTAAAAAATGAGCTGATTTAACAA
AAATTTAACGCGAATTTTAACAAAATATTAACGTTTATAATTTCA
GGTGGCATCTTTCGGGGAAATGTGCGCGGAACCCCTATTTGTTTA
TTTTTCTAAATACATTCAAATATGTATCCGCTCATGAGACAATAA
CCCTGATAAATGCTTCAATAATATTGAAAAAGGAAGAGTATGAGT
ATTCAACATTTCCGTGTCGCCCTTATTCCCTTTTTTGCGGCATTT
TGCCTTCCTGTTTTTGCTCACCCAGAAACGCTGGTGAAAGTAAAA
GATGCTGAAGATCAGTTGGGTGCACGAGTGGGTTACATCGAACTG
GATCTCAATAGTGGTAAGATCCTTGAGAGTTTTCGCCCCGAAGAA
CGTTTTCCAATGATGAGCACTTTTAAAGTTCTGCTATGTGGCGCG
GTATTATCCCGTATTGACGCCGGGCAAGAGCAACTCGGTCGCCGC
ATACACTATTCTCAGAATGACTTGGTTGAGTACTCACCAGTCACA
GAAAAGCATCTTACGGATGGCATGACAGTAAGAGAATTATGCAGT
GCTGCCATAACCATGAGTGATAACACTGCGGCCAACTTACTTCTG
ACAACGATCGGAGGACCGAAGGAGCTAACCGCTTTTTTGCACAAC
ATGGGGGATCATGTAACTCGCCTTGATCGTTGGGAACCGGAGCTG
AATGAAGCCATACCAAACGACGAGCGTGACACCACGATGCCTGTA
GTAATGGTAACAACGTTGCGCAAACTATTAACTGGCGAACTACTT
ACTCTAGCTTCCCGGCAACAATTAATAGACTGGATGGAGGCGGAT
AAAGTTGCAGGACCACTTCTGCGCTCGGCCCTTCCGGCTGGCTGG
TTTATTGCTGATAAATCTGGAGCCGGTGAGCGTGGGTCTCGCGGT
ATCATTGCAGCACTGGGGCCAGATGGTAAGCCCTCCCGTATCGTA
GTTATCTACACGACGGGGAGTCAGGCAACTATGGATGAACGAAAT
AGACAGATCGCTGAGATAGGTGCCTCACTGATTAAGCATTGGTAA
CTGTCAGACCAAGTTTACTCATATATACTTTAGATTGATTTAAAA
CTTCATTTTTAATTTAAAAGGATCTAGGTGAAGATCCTTTTTGAT
AATCTCATGACCAAAATCCCTTAACGTGAGTTTTCGTTCCACTGA
GCGTCAGACCCCGTAGAAAAGATCAAAGGATCTTCTTGAGATCCT
TTTTTTCTGCGCGTAATCTGCTGCTTGCAAACAAAAAAACCACCG
CTACCAGCGGTGGTTTGTTTGCCGGATCAAGAGCTACCAACTCTT
TTTCCGAAGGTAACTGGCTTCAGCAGAGCGCAGATACCAAATACT
GTCCTTCTAGTGTAGCCGTAGTTAGGCCACCACTTCAAGAACTCT
GTAGCACCGCCTACATACCTCGCTCTGCTAATCCTGTTACCAGTG
GCTGCTGCCAGTGGCGATAAGTCGTGTCTTACCGGGTTGGACTCA
AGACGATAGTTACCGGATAAGGCGCAGCGGTCGGGCTGAACGGGG
GGTTCGTGCACACAGCCCAGCTTGGAGCGAACGACCTACACCGAA
CTGAGATACCTACAGCGTGAGCTATGAGAAAGCGCCACGCTTCCC
GAAGGGAGAAAGGCGGACAGGTATCCGGTAAGCGGCAGGGTCGGA
ACAGGAGAGCGCACGAGGGAGCTTCCAGGGGGAAACGCCTGGTAT
CTTTATAGTCCTGTCGGGTTTCGCCACCTCTGACTTGAGCGTCGA
TTTTTGTGATGCTCGTCAGGGGGGCGGAGCCTATGGAAAAACGCC
AGCAACGCGGCCTTTTTACGGTTCCTGGCCTTTTGCTGCGGTTTT
GCTCACATGTTCTTTCCTGCGTTATCCCCTGATTCTGTGGATAAC
CGTATTACCGCCTTTGAGTGAGCTGATACCGCTCGCCGCAGCCGA
ACGACCGAGCGCAGCGAGTCAGTGAGCGAGGAAGCGGAAGAGCGC
CCAATACGCAAACCGCCTCTCCCCGCGCGTTGGCCGATTCATTAA
TGCAGCTGGCACGACAGGTTTCCCGACTGGAAAGCGGGCAGTGAG
CGCAACGCAATTAATGTGAGTTAGCTCACTCATTAGGCACCCCAG
GCTTTACACTTTATGCTTCCGGCTCGTATGTTGTGTGGAATTGTG
AGCGGATAACAATTTCACACAGGAAACAGCTATGACCATGATTAC
GCCAGATTTAATTAAGGCTGCGCGCTCGCTCGCTCACTGAGGCCG
CCCGGGCAAAGCCCGGGCGTCGGGCGACCTTTGGTCGCCCGGCCT
CAGTGAGCGAGCGAGCGCGCAGAGAGGGAGTGGCCAACTCCATCA
CTAGGGGTTCCTTGTAGTTAATGATTAACCCGCCATGCTACTTAT
CTACGTAGCCATGCTCTAGGAAGATCGGAATTCGCCCTTAAGCTA
GCTAGTTATTAATAGTAATCAATTACGGGGTCATTAGTTCATAGC
CCATATATGGAGTTCCGCGTTACATAACTTACGGTAAATGGCCCG
CCTGGCTGACCGCCCAACGACCCCCGCCCATTGACGTCAATAATG
ACGTATGTTCCCATAGTAACGCCAATAGGGACTTTCCATTGACGT
CAATGGGTGGAGTATTTACGGTAAACTGCCCACTTGGCAGTACAT
CAAGTGTATCATATGCCAAGTACGCCCCCTATTGACGTCAATGAC
GGTAAATGGCCCGCCTGGCATTATGCCCAGTACATGACCTTATGG
GACTTTCCTACTTGGCAGTACATCTACGTATTAGTCATCGCTATT
ACCATGGTGATGCGGTTTTGGCAGTACATCAATGGGCGTGGATAG
CGGTTTGACTCACGGGGATTTCCAAGTCTCCACCCCATTGACGTC
AATGGGAGTTTGTTTTGGCACCAAAATCAACGGGACTTTCCAAAA
TGTCGTAACAACTCCGCCCCATTGACGCAAATGGGCGGTAGGCGT
GTACGGTGGGAGGTCTATATAAGCAGAGCTCTCTGGCTAACTAAG
CCGCAGCCATGAACGGCACAGAGGGCCCCAATTTTTATGTGCCCT
TCTCCAACGTCACAGGCGTGGTGCGGAGCCCCTTCGAGCAGCCGC
AGTACTACCTGGCGGAACCATGGCAGTTCTCCATGCTGGCAGCGT
ACATGTTCCTGCTCATCGTGCTGGGCTTCCCCATCAACTTCCTCA
CGCTCTACGTCACCGTACAGCACAAGAAGCTGCGCACACCCCTCA
ACTACATCCTGCTCAACTTGGCCGTGGCTGACCTCTTCATGGTCT
TCGGAGGATTCACCACCACCCTCTACACATCACTCCATGGCTACT
TCGTCTTTGGGCCCACAGGCTGTAATCTCGAGGGCTTCTTTGCCA
CACTTGGAGGTGAAATCGCCCTGTGGTCCCTGGTGGTCCTGGCCA
TTGAGCGCTACGTGGTGGTCTGCAAGCCGATGAGCAACTTCCGCT
TCGGGGAGAATCACGCTATCATGGGTGTGGTCTTCACCTGGATCA
TGGCGTTGGCCTGTGCTGCTCCCCCACTCGTTGGCTGGTCCAGGT
ACATCCCTGAGGGCATGCAATGTTCATGCGGGATTGACTACTACA
CACTCAAGCCTGAGGTCAACAACGAATCCTTTGTCATCTACATGT
TCGTGGTCCACTTCACCATTCCTATGATCGTCATCTTCTTCTGCT
ATGGGCAGCTGGTCTTCACAGTCAAGGAGGCGGCTGCCCAGCAGC
AGGAGTCAGCCACCACTCAGAAGGCAGAGAAGGAAGTCACCCGCA
TGGTTATCATCATGGTCATCTTCTTCCTGATCTGCTGGCTTCCCT
ACGCCAGTGTGGCCTTCTACATCTTCACCCACCAGGGCTCCAACT
TCGGCCCCATCTTCATGACTCTGCCAGCTTTCTTTGCTAAGAGCT
CTTCCATCTATAACCCGGTCATCTACATCATGTTGAACAAGCAGT
TCCGGAACTGTATGCTCACCACGCTGTGCTGCGGCAAGAATCCAC
TGGGAGATGACGACGCCTCTGCCACCGCTTCCAAGACGGAGACCA
GCCAGGTGGCTCCAGCCTAACGCGTCCCCACGGGCTCTTCGTAGA
CAGAGCCGCAGCCATGGTGAGCAAGGGCGAGGAGCTGTTCACCGG
GGTGGTGCCCATCCTGGTCGAGCTGGACGGCGACGTAAACGGCCA
CAAGTTCAGCGTGTCCGGCGAGGGCGAGGGCGATGCCACCTACGG
CAAGCTGACCCTGAAGTTCATCTGCACCACCGGCAAGCTGCCCGT
GCCCTGGCCCACCCTCGTGACCACCCTGACCTACGGCGTGCAGTG
CTTCAGCCGCTACCCCGACCACATGAAGCAGCACGACTTCTTCAA
GTCCGCCATGCCCGAAGGCTACGTCCAGGAGCGCACCATCTTCTT
CAAGGACGACGGCAACTACAAGACCCGCGCCGAGGTGAAGTTCGA
GGGCGACACCCTGGTGAACCGCATCGAGCTGAAGGGCATCGACTT
CAAGGAGGACGGCAACATCCTGGGGCACAAGCTGGAGTACAACTA
CAACAGCCACAACGTCTATATCATGGCCGACAAGCAGAAGAACGG
CATCAAGGTGAACTTCAAGATCCGCCACAACATCGAGGACGGCAG
CGTGCAGCTCGCCGACCACTACCAGCAGAACACCCCCATCGGCGA
CGGCCCCGTGCTGCTGCCCGACAACCACTACCTGAGCACCCAGTC
CGCCCTGAGCAAAGACCCCAACGAGAAGCGCGATCACATGGTCCT
GCTGGAGTTCGTGACCGCCGCCGGGATCACTCTCGGCATGGACGA
GCTGTACAAGTAATAAGGCCTGCTGCCGGCTCTGCGGCCTCTTCC
GCGTCTTCGAGATCTGCCTCGACTGTGCCTTCTAGTTGCCAGCCA
TCTGTTGTTTGCCCCTCCCCCGTGCCTTCCTTGACCCTGGAAGGT
GCCACTCCCACTGTCCTTTCCTAATAAAATGAGGAAATTGCATCG
CATTGTCTGAGTAGGTGTCATTCTATTCTGGGGGGTGGGGTGGGG
CAGGACAGCAAGGGGGAGGATTGGGAAGACAATAGCAGGCATGCT
GGGGACTCGAGTTAAGGGCGAATTCCCGATAAGGATCTTCCTAGA
GCATGGCTACGTAGATAAGTAGCATGGCGGGTTAATCATTAACTA
CAAGGAACCCCTAGTGATGGAGTTGGCCACTCCCTCTCTGCGCGC
TCGCTCGCTCACTGAGGCCGGGCGACCAAAGGTCGCCCGACGCCC
GGGCTTTGCCCGGGCGGCCTCAGTGAGCGAGCGAGCGCGCAGCCT
T
(see FIG. 28 for a plasmid map).

For packaging AAV8 virus, pAAV rep/Cap 2/2, 2/8, 2/7m8, and Adenovirus helper plasmids were used.

Primers

| Name | Sequence (5′→3′) | Purpose |
|---|---|---|
| RHO gRNA1-Sp F | CACCGCTGTCTACGAA | Construct gRNA1 for SpCas9 |
| | GAGCCCGTG (SEQ ID | targeting RHO ATG upstream |
| | NO: 4) | |
| RHO gRNA1-Sp R | AAACCACGGGCTCTTC | |
| | GTAGACAGC (SEQ ID | |
| | NO: 5) | |
| RHO gRNA2-Sp F | CACCCGTTCATGGCTG | Construct gRNA2 for SpCas9 |
| | CGGCTCTCG (SEQ ID | targeting RHO ATG upstream |
| | NO: 6) | |
| RHO gRNA2-Sp R | AAACCGAGAGCCGCA | RHOP23H/wt mouse genotyping |
| | GCCATGAACC | |
| | (SEQ ID NO: 7) | |
| mRHO exon 1 F | TGATATCTCGCGGATG | RHO^{-/-} mouse genotyping |
| | CTGAAT | |
| | (SEQ ID NO: 8) | |
| mRHO exon 1 R | TGGGCCTTTAGATGAG | RHO-HITI donor construction |
| | ACCAA | |
| | (SEQ ID NO: 9) | |
| RHO_Neo F | CGGGAGCGGCGATAC | Construct pCMV-Kozak- |
| | CGTAAAGC | GFP-Stop-Kozak-RHO-Stop |
| | (SEQ ID NO: 10) | |
| RHO_Neo R | GAAGCGGGAAGGGAC | |
| | TGGCTGCTA | |
| | (SEQ ID NO: 11) | |
| RHO CDS F | AAACGCGACGCGTCC | |
| | CCACGGGCTCTTCGTA | |
| | GACAGAGCCGCAGCC | |
| | ATGAACGGCACAGAG | |
| | GGCCCCAATTTTTATG | |
| | (SEQ ID NO: 12) | |
| RHO CDS R | CCAGGTGGCTCCAGCC | |
| | TAATAAGTCGACCCCC | |
| | ACGGGCTCTTCGTAGA | |
| | CAGGC | |
| | (SEQ ID NO: 13) | |
| AgeI GFP F: | GTGAACACCGGTAGA | |
| | CAGAGCCGCAGCCAT | |
| | GGTGAGCAAGGGCGA | |
| | GG (SEQ ID NO: 14) | |
| SalI GFP R: | TTGGCGCGCCGTCGAC | |
| | TTATTACTTGTACAGC | |
| | TCGTCCATGC (SEQ ID | |
| | NO: 15) | |
| Kz-RHO CDS- | GAGGTCTATATAAGCA | Construct pCMV-Kozak- |
| stop F | GAGCTCTCTGGCTAAC | RHO-Stop-Kozak-GFP-Stop |
| | TAAGCCGCAGCCATG | |
| | AACGGCACAGAG (SEQ | |
| | ID NO: 16) | |
| Kz-RHO CDS- | GCGGCTCTGTCTACGA | |
| stop R | AGAGCCCGTGGGGAC | |
| | GCGTTAGGCTGGAGCC | |
| | ACCTGGCTGGTC (SEQ | |
| | ID NO: 17) | |
| RHO KI_F | GCTGAGCTCGCCAAGC | RHO KINGS sequencing |
| | AGCCTTGGT (SEQ ID | |
| | NO: 18) | |
| RHO KI_R | CATGTACGCTGCCAGC | |
| | ATGGAGAAC (SEQ ID | |
| | NO: 19) | |

Cell culture and transfection. Both HEK293T and MEF cells were cultured in DMEM, 10% (v/v) FBS, 1% (v/v) 4 Penicillin/Streptomycin (P/S) media under 37° C., 5% CO₂ incubator. Cells were trypsinized and split with a ratio of 1:5 to 1:10 after reaching 90-95% confluency every 2-3 days. Plasmids were transfected into HEK293T and MEF cells by using 1 (mg/ml) polyethyleneimine (PEI) or LIPOFECTAMINE™ 3000. The PEI or LIPOFECTAMINE™ 3000 was mixed with plasmid in ratio 3:1 in DMEM medium (20011.1/1 μg plasmid) and incubated 10-15 mins at room temperature. The mixture was subsequently added into HEK293T or MEF cells with DMEM, 10% (v/v) FBS, 1% (v/v) Penicillin/Streptomycin medium and placed in 37° C., 5% CO₂ incubator. The old medium was removed and replaced with the fresh DMEM 24 hours after transfection.

Subretinal injection of AAV. New-born mouse pups were anesthetized by hypothermia in ice for 2-3 mins. An incision was made in the eyelid to expose the eyeball. AAV-hRK-Cas9 and AAV-hRK-18 mCherry-pU6-gRNA-donor viruses were mixed in PBS to a final concentration of 5E12 vg/ml for each virus. 0.25 11.1 of virus mix was injected into the subretinal space using a pulled angled glass pipette controlled by a FEMTOJET® (EPPENDORF™). The right eye of the animal was injected, and the left eye was uninjected for within animal controls.

Fluorescence-activated cell sorting (FACS) of transduced cells. The transfected MEF cells with mCherry marker were trypsinized and sorted by FACS at three days after transfection. The photoreceptors were labeled by subretinally-delivered AAV-hRK-mCherry in neonatal mice. At 14 days after injection, eyes were enucleated for retina dissection and disassociation by using papain solution. The mCherry+ and/or GFP+ positive cells were sorted and collected by Sony SH800 Cell Sorter.

DNA extraction. Genomic DNA (gDNA) was extracted by using the PURELINK™ Genomic DNA Mini Kit (Thermo Fisher Scientific). Briefly, the cells or tissues were collected and rinsed two times with PBS. After rinsing, cells/tissues were treated with DNA lysis buffer containing protease K and RNAase in 55° C. for 1 hour or until pellets were dissolved. Next, the cell lysis mixture was thoroughly mixed with DNA binding buffer and ethanol before flowing through the binding column by centrifugation at 10,000 g, 1 min. After binding, the column was washed two times with 500 μl of washing buffer. Finally, the gDNA was recovered in 50 μl elution buffer and kept at −20° C. for further analysis.

Tracking of indel by decomposition (TIDE) analysis. Tracking of indel by decomposition (TIDE) is a rapid and reliable assessment for CRISPR/Cas9 gene editing efficiency developed by Brinkman et al., 2014. TIDE uses the quantitative sequence trace data from conventional Sanger sequencing results to estimate the frequency of insertions and deletions (INDELs), or editing efficiency, and identify the predominant INDEL types in the DNA sequences. The region around the CRISPR/Cas9 targeting site was amplified from the gDNA samples by using a specific primer pair and a high-fidelity DNA polymerase. In some aspects, the amplified fragment is around 700 bp, and the targeted site is located 200 bp downstream from the sequencing start site. In this study, PHUSION® HF DNA polymerase (NEB-M0530L) was used to amplify the DNA fragments. The primers used for TIDE analysis are listed in the primer table. The amplification is as follows: 98° C. for 5 mins, 25 cycles of (98° C. for 30 secs, 65° C. for 20 secs, 72° C. for 20 secs), 72° C. for 10 mins, 4° C. holding. PCR products were sequenced by the Sanger method performed. The sequencing results were subsequently uploaded to the webtool TIDE (accessible at tide.deskgen.com) or ICE CRISPR Analysis (accessible at ice. synthego.com) for analyzing the editing efficiency.

AAV packaging and titration. Recombinant AAV8 vectors were produced in HEK293T cells, and the AAV purification process is based on the iodixanol gradient method. 3 The HEK293T cells were transfected at 80-90% confluency. For 5 plates (150 mm) of HEK293T cells, 35 μg of pAAV vector transgene plasmid, 35 μg of rep/cap packaging plasmid, and 100 μg adenoviral helper plasmid were mixed with 510 μg of polyethyleneimine (PEI) (DNA:PEI=1:3) in DMEM. The mixture was incubated 15 mins at room temperature before adding to the HEK293T cells cultured in DMEM, 10% (v/v) NU-SERUM™ (355500, CORNING®), 1% (v/v) P/S. After 24 hours, the old medium was changed with fresh DMEM, 1% (v/v) P/S. 72 hours after transfection, the AAV8 supernatant was collected and centrifuged at 2,000 g, 15 mins, 4° C., to remove cell debris. After cell debris clarification, the collected supernatant was mixed with 8.5% (w/v) PEG-8000, 0.4 M NaCl, at 4° C. in 2 hours, to precipitate the AAV virus. The precipitation particles were then collected by centrifugation at 7,000×g for 10 mins, 4° C., and resuspended in the virus lysis buffer (150 mM NaCl and 20 mM Tris, pH 8.0). The virus mixture was further purified by ultra-centrifugation in the iodixanol gradient at 147,000×g at 4° C. for 90 mins. The AAV residing at 40% iodixanol fraction was collected and washed three times with PBS by using AMICON® 100K columns (EMD Millipore). About 200 μl of final volume AAV was collected and stored at −80° C. Virus titration was performed by protein SDS-PAGE method.

Optomotor and Electroretinography (ERG). Mouse visual acuity was measured as maximal spatial frequency by optomotor test in THE OptoMotry System (CerebralMechanics). Mouse visual acuity was examined without knowing which AAVs were injected to avoid the bias. The photopic vision was tested at a background light of ˜70 cd/m², and the contrast of the grates was set at 100%. The mice being tested were placed on a platform surrounded by four screens. Vertical black and white grating at a defined spatial frequency was displayed on the screens and rotated clockwise or counterclockwise. Via the camera screen, the vision of mice was checked according to whether they moved along with the grating rotation (˜5 seconds per episode). A series of test episodes was performed in the program to determine the maximum spatial frequency that the left eye and the right eye of the mice can see.

The mices' eye physiology was determined by ERG measurements using the Espion E3 22 System (Diagnosys LLC). The ERG protocol was developed from the previous studies of wide type mice to characterize the rod and cone responses.^{4,5} The mice were adapted in a dark cabinet overnight before ERG testing, then were anesthetized with a ketamine/xylazine (100/10 mg/kg) mixture. The eyes were dilated with a drop of 5% phenylephrine and 0.5% tropicamide solution for 5 mins and were kept hydrated with corneal gel. After putting mice on the platform, gold-wire electrodes for measuring electrical responses were placed on each eye cornea, while a reference electrode and a ground electrode were put into the mouth and the tail, respectively. All these steps were performed in the darkroom under dim red light. For scotopic ERG recordings, a multiple 530 nm light with different intensities (increments from 0.01 cd s/m² to 30 7 cd·s/m²) were elicited to stimulate scotopic responses in a specific time interval. For photopic ERG recordings, 5 mins exposure under 10 cd·s/m² light intensity was adopted to inhibit the rod function. The photopic response was measured by multiple flashes of 30 cd·s/m² intensity in the illuminated background (10 cd·s/m²). The average amplitude and implicit time of B-wave was recorded and exported for further analysis.

Optical Coherence Tomography (OCT). In vivo OCT was performed using a Bioptigen spectral domain optical coherence tomographer (SD-OCT, Bioptigen Envisu R4310 SD-OCT, Germany). Before the procedure, mice were anaesthetized by intraperitoneal injection with a standard mixture of ketamine/xylazine (100 mg ketamine+10 mg xylazine)/kg body weight. Mice were provided supplemental indirect warmth by a heating pad during anesthesia. Cornea and pupil were anaesthetized and dilated with instillation of 0.5% proxymetacaine hydrochloride (Provain-POS, Germany), and 0.5% tropicamide and 0.5% phenylephrine hydrochloride (Mydrin-P, Santen Pharmaceutical Co., Japan) solution. During retinal imaging, cornea was hydrated with lubricating eye drops (SYSTANE® ULTRA, Alcon). To image the retina, the volume intensity projection was centered on the optic nerve, and the following scan parameters were applied: radial volume scans 1.7 mm in diameter, 1000 A-scans/B-scan, 8 B-scans/volume, 24 frame/B-scan, 80 inactive A-scans/B-scan, and 1 volume. Retinal thickness was further measured using ImageJ.

Retinal Section and histology. Mice were sacrificed by CO₂ euthanasia and cervical dislocation. The eyeballs were dorsally marked before enucleation, and retinas were dissected and fixed in 4% formaldehyde for 30 mins at room temperature. Fixed retinas were washed three times with PBS and sequentially cryoprotected in 5%, 15%, and 30% sucrose for 15, 30, and 60 mins, respectively. Next, the eyecups were soaked in optimal cutting temperature (OCT) and 30% sucrose solution 1:1 at 4° C. overnight and subsequently embedded in cryomold in an orientation to achieve dorsal-ventral oriented slices or infected-uninfected oriented slices. After tissue freezing below −20° C., a series of 20 μm sections were cut and collected on the glass slides using a cryostat machine (EPREDIA™ Microm HM525 NX Cryostat, Thermo Fisher). In the immunostaining process, the retinal sections or whole retinal cups were incubated in blocking solution (3% BSA, 0.1% Triton X-100 in PBS-PBST) for 30 mins, then were covered with primary antibodies at the recommended dilution at 4° C., overnight. After primary antibody incubation, the samples were washed three times with PB ST before incubation in a mixture of DAPI (0.5 μg/ml) and secondary antibodies in the dark for 2 hours at room temperature. The primary antibodies and secondary antibodies are listed in the following table. The sections or whole flat-mount retinas were mounted with an anti-fade solution before microscopy processing or cool storage. Slide images were captured in Zeiss LSM780 confocal microscope or Nikon ECLIPSE Ni-E upright microscope. The histology measurements and image processing were performed in ImageJ software.

Antibodies

| | | | Working | | |
|---|---|---|---|---|---|
| Name | Type | Host | Dilution | Brand (Cat.) | Purpose |
| mCAR | 1° anti- | Rabbit | IHC | Millipore | Label cone |
| | body | | (1:500) | (AB15282) | cells |
| RHO | 1° anti- | Mouse | IHC | Millipore | Label rod |
| 4D2 | body | | (1:500) | (MABN15) | outer |
| | | | | | segments |
| Anti- | 2° anti- | Donkey | IHC | Jackson | Label mouse |
| Mouse | body | | (1:1000) | ImmunoRe- | antibody |
| | Alexa | | | search (715- | with green |
| | 488 | | | 545-150) | fluorescence |
| Anti- | 2° anti- | Donkey | IHC | Jackson | Label mouse |
| Mouse | body | | (1:1000) | ImmunoRe- | antibody |
| | Alexa | | | search (715- | with magenta |
| | 647 | | | 605-150) | fluorescence |
| Anti- | 2° anti- | Donkey | IHC | Jackson | Label rabbit |
| Rabbit | body | | (1:1000) | ImmunoRe- | antibody |
| | Alexa | | | search (711- | with green |
| | 488 | | | 545-152) | fluorescence |
| Anti- | 2° anti- | Donkey | IHC | Jackson | Label rabbit |
| Rabbit | body | | (1:1000) | ImmunoRe- | antibody |
| | Alexa | | | search (711- | with magenta |
| | 647 | | | 605-152) | fluorescence |

NGS analysis. To quantify the RHO KI and INDEL frequency in retinas of RHO^{P23H/wt} mice receiving 5′ UTR RHO KI, NGS was performed to query the sequence at the target site. In brief, the mCherry+ cells were sorted and collected by Sony SH800 Cell Sorter. gDNA was extracted by using the PURELINK™ Genomic DNA Mini Kit (Thermo Fisher Scientific). A pair of primers (RHO KI F: 5′-GCTGAGCTCGCCAAGCAGCCTTGGT-3′ (SEQ ID NO:19); RHO KI R: 5′-CATGTACGCTGCCAGCATGGAGAAC-3′ (SEQ ID NO:20)) flanking the target site was designed to amplify the extracted gDNA. PCR product was sequenced using the Illumina NovaSeq 6000 platform and analyzed by CRISPResso.

Statistical Analysis. Data were presented as mean±s.e.m. Sample sizes are indicated for each experiment. One-way or two-way ANOVA analysis followed by Tukey test was performed to compare multiple groups, and the unpaired two-tailed Student's t-test was used to compare two groups.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1. Lem, J. et al. Morphological, physiological, and biochemical changes in rhodopsin knockout mice. Proc. Natl. Acad. Sci. U.S.A 96, 736-741 (1999).
2. Sakami, S. et al. Probing mechanisms of photoreceptor degeneration in a new mouse model of the common form of autosomal dominant retinitis pigmentosa due to P23H opsin mutations. J. Biol. Chem. 286, 10551-10567 (2011).
3. Grieger, J. C., Choi, V. W. & Samulski, R. J. Production and characterization of adeno-associated viral vectors. Nat. Protoc. 1, 1412-1428 (2006).
4. Goto, Y. An electrode to record the mouse cornea electroretinogram. Doc. Ophthalmol. 91, 147-154 (1996).
5. Kinoshita, J. and Peachey, N. S. Noninvasive Electroretinographic Procedures for the Study of the Mouse Retina. Curr Protoc Mouse Biol. 8, 1-16 (2018).

1. A method for editing the genome of a cell, the method comprising contacting the cell with a composition comprising a nuclease and an exogenous nucleic acid encoding a knock-in cassette comprising a coding sequence for a wild-type gene and translation initiation and termination elements for expression of the wild-type gene,
wherein the nuclease causes a break within a 5′ UTR of an endogenous nucleic acid encoding a mutated variant of the wild-type gene,
wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and
wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid.

2. The method of claim 1, wherein:
integration of the exogenous nucleic acid encoding the knock-in cassette results in expression of the wild-type gene by the cell; and/or
expression of the wild-type gene by the cell inhibits expression of the mutated variant of the wild-type gene.

3. The method of claim 1, wherein:
the nuclease is encoded by the same exogenous nucleic acid encoding the knock-in cassette, and wherein the exogenous nucleic acid is comprised in a vector; or
the nuclease is encoded by a nucleic acid that is different from the exogenous nucleic acid encoding the knock-in cassette, and wherein the nucleic acid encoding the nuclease and the exogenous nucleic acid encoding the knock-in cassette are comprised in two different vectors.

4. The method of claim 3, wherein the nuclease is a CRISPR/Cas nuclease, and wherein the vector comprising the exogenous nucleic acid encoding the knock-in cassette further comprises a nucleic acid encoding a guide molecule for the CRISPR/Cas nuclease.

5. The method of claim 3, wherein the vector(s) are plasmids, transposons, cosmids, artificial chromosomes, lipid nanoparticles, viral vectors, or a combination thereof.

6. The method of claim 1, wherein:
the mutated variant of the wild-type gene is a dominant variant, and wherein the wild-type gene is the RHO gene; or
the mutated variant of the wild-type gene is a recessive variant.

7. An engineered cell comprising a genomic modification, wherein the genomic modification comprises integration of an exogenous nucleic acid encoding a knock-in cassette into the genome of the cell according to the method of claim 1.

8. A composition comprising:
a nuclease; and
an exogenous nucleic acid encoding a knock-in cassette comprising a coding sequence for a wild-type gene and translation initiation and termination elements for expression of the wild-type gene;
wherein, when introduced into a cell, the nuclease causes a break within a 5′ UTR of an endogenous nucleic acid encoding a mutated variant of the wild-type gene,
wherein the endogenous nucleic acid encodes, in the 5′ to 3′ direction, the 5′ UTR, a translation initiation element for expression of the mutated variant of the wild-type gene, and a coding sequence for the mutated variant of the wild-type gene, and
wherein the exogenous nucleic acid encoding the knock-in cassette is integrated by homology-independent targeted integration into the 5′ UTR of the endogenous nucleic acid upstream (5′) of the translation initiation element encoded by the endogenous nucleic acid.

9. The composition of claim 8, wherein:
the nuclease is encoded by the same exogenous nucleic acid encoding the knock-in cassette, and wherein the exogenous nucleic acid is comprised in a vector; or
the nuclease is encoded by a nucleic acid that is different from the exogenous nucleic acid encoding the knock-in cassette, and wherein the nucleic acid encoding the nuclease and the exogenous nucleic acid encoding the knock-in cassette are comprised in two different vectors.

10. The composition of claim 9, wherein the nuclease is a CRISPR/Cas nuclease, and wherein the vector comprising the exogenous nucleic acid encoding the knock-in cassette further comprises a nucleic acid encoding a guide molecule for the CRISPR/Cas nuclease.

11. The composition of claim 9, wherein the vector(s) are plasmids, transposons, cosmids, artificial chromosomes, lipid nanoparticles, viral vectors, or a combination thereof.

12. The composition of claim 8, wherein:
the mutated variant of the wild-type gene is a dominant variant, and the wild-type gene is the RHO gene; or
the mutated variant of the wild-type gene is a recessive variant.

13. (canceled)

14. A method of expressing a wild-type gene in cells and/or decreasing the expression of a mutated variant of a wild-type gene in cells, the method comprising introducing the composition of claim 8 into the cells.

15. A method for treating or preventing an autosomal disorder in a subject identified as expressing a mutated gene variant, the method comprising introducing into a cell of the subject an effective amount of a composition comprising:
a Cas nuclease; and
an exogenous nucleic acid encoding a knock-in cassette comprising a coding sequence for a wild-type gene and translation initiation and termination elements for expression of the wild-type gene;
wherein the Cas nuclease causes a break within a 5′ UTR of an endogenous nucleic acid encoding the mutated gene variant,
wherein the endogenous nucleic acid encodes, in the 5′ to 3′ direction, the 5′ UTR, a translation initiation element for expression of the mutated gene variant, and a coding sequence for the mutated gene variant,
wherein the nucleic acid encoding the knock-in cassette is integrated by homology-independent targeted integration into the 5′ UTR of the endogenous nucleic acid upstream (5′) of the translation initiation element encoded by the endogenous nucleic acid,
wherein integration of the nucleic acid encoding the knock-in cassette results in expression of the wild-type gene, and
wherein expression of the wild-type gene results in decreased expression of the mutated gene variant.

16. The method of claim 15, wherein:
the exogenous nucleic acid encoding the knock-in cassette is not integrated in-frame with the endogenous nucleic acid encoding the mutated gene variant; or
the exogenous nucleic acid encoding the knock-in cassette is integrated in-frame with the endogenous nucleic acid encoding the mutated variant of the wild-type gene.

17. The method of claim 15, wherein:
the Cas nuclease is encoded by the same exogenous nucleic acid encoding the knock-in cassette, and wherein the exogenous nucleic acid is comprised in a vector; or
the Cas nuclease is encoded by a nucleic acid that is different from the exogenous nucleic acid encoding the knock-in cassette, and wherein the nucleic acid encoding the Cas nuclease and the exogenous nucleic acid encoding the knock-in cassette are comprised in two different vectors.

18. The method of claim 17, wherein the vector comprising the exogenous nucleic acid encoding the knock-in cassette further comprises a nucleic acid encoding a guide molecule for the Cas nuclease.

19. The method of claim 17, wherein the vector(s) are plasmids, transposons, cosmids, artificial chromosomes, lipid nanoparticles, viral vectors, or a combination thereof.

20. The method of claim 15, wherein:
the autosomal disorder is an autosomal dominant disorder, and the mutated gene variant is a dominant variant; or
the autosomal disorder is an autosomal recessive disorder, and the mutated gene variant is a recessive variant.

21. The method of claim 15, wherein integration of the nucleic acid encoding the knock-in cassette is not enriched by drug selection.


SEQUENCE LISTING (36 sequences)

SEQ ID NO: 1 - DNA, 5385 bp, Homo sapiens
  source 1..5385: mol_type = genomic DNA
ggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctctggcatcctctgcctcccctctcagcccctgtcctcaggtgcccctccagcctccctgccgcgttccaagtctcctggtgttgagaaccgcaagcagccgctctgaagcagttcctttttgctttagaataatgtcttgcatttaacaggaaaacagatggggtgctgcagggataacagatcccacttaacagagaggaaaactgaggcagggagaggggaagagactcatttagggatgtggccaggcagcaacaagagcctaggtctcctggctgtgatccaggaatatctctgctgagatgcaggaggagacgctagaagcagccattgcaaagctgggtgacggggagagcttaccgccagccacaagcgtctctctgccagccttgccctgtctcccccatgtccaggctgctgcctcggtcccattctcagggaatctctggccattgttgggtgtttgttgcattcaataatcacagatcactcagttctggccagaaggtgggtgtgccacttacgggtggttgttctctgcagggtcagtcccagtttacaaatattgtccctttcactgttaggaatgtcccagtttggttgattaactatatggccactctccctatggaacttcatggggtggtgagcaggacagatgtctgaattccatcatttccttcttcttcctctgggcaaaacattgcacattgcttcatggctcctaggagaggcccccacatgtccgggttatttcatttcccgagaagggagagggaggaaggactgccaattctgggtttccaccacctctgcattccttcccaacaaggaactctgccccacattaggatgcattcttctgctaaacacacacacacacacacacacacacaacacacacacacacacacacacacacacacacacaaaactccctaccgggttcccagttcaatcctgaccccctgatctgattcgtgtcccttatgggcccagagcgctaagcaaataacttcccccattccctggaatttctttgcccagctctcctcagcgtgtggtccctctgccccttccccctcctcccagcaccaagctctctccttccccaaggcctcctcaaatccctctcccactcctggttgccttcctagctaccctctccctgtctaggggggagtgcaccctccttaggcagtggggtctgtgctgaccgcctgctgactgccttgcaggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtaatggcactgagcagaagggaagaagctccgggggctctttgtagggtcctccagtcaggactcaaacccagtagtgtctggttccaggcactgaccttgtatgtctcctggcccaaatgcccactcagggtaggggtgtagggcagaagaagaaacagactctaatgttgctacaagggctggtcccatctcctgagccccatgtcaaacagaatccaagacatcccaacccttcaccttggctgtgcccctaatcctcaactaagctaggcgcaaattccaatcctctttggtctagtaccccgggggcagccccctctaaccttgggcctcagcagcaggggaggccacaccttcctagtgcaggtggccatattgtggccccttggaactgggtcccactcagcctctaggcgattgtctcctaatggggctgagatgagacacagtggggacagtggtttggacaataggactggtgactctggtccccagaggcctcatgtccctctgtctccagaaaattcccactctcacttccctttcctcctcagtcttgctagggtccatttcttaccccttgctgaatttgagcccaccccctggactttttccccatcttctccaatctggcctagttctatcctctggaagcagagccgctggacgctctgggtttcctgaggcccgtccactgtcaccaatatcaggaaccattgccacgtcctaatgacgtgcgctggaagcctctagtttccagaagctgcacaaagatcccttagatactctgtgtgtccatctttggcctggaaaatactctcaccctggggctaggaagacctcggtttgtacaaacttcctcaaatgcagagcctgagggctctccccacctcctcaccaaccctctgcgtggcatagccctagcctcagcgggcagtggatgctggggctgggcatgcagggagaggctgggtggtgtcatctggtaacgcagccaccaaacaatgaagcgacactgattccacaaggtgcatctgcatccccatctgatccattccatcctgtcacccagccatgcagacgtttatgatccccttttccagggagggaatgtgaagccccagaaagggccagcgctcggcagccaccttggctgttcccaagtccctcacaggcagggtctccctacctgcctgtcctcaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggtacgggccggggggtgggcggcctcacggctctgagggtccagcccccagcatgcatctgcggctcctgctccctggaggagccatggtctggacccgggtcccgtgtcctgcaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcaggtgcctactgcgggtgggagggccccagtgccccaggccacaggcgctgcctgccaaggacaagctacttcccagggcaggggagggggctccatcagggttactggcagcagtcttgggtcagcagtcccaatggggagtgtgtgagaaatgcagattcctggccccactcagaactgctgaatctcagggtgggcccaggaacctgcatttccagcaagccctccacaggtggctcagatgctcactcaggtgggagaagctccagtcagctagttctggaagcccaatgtcaaagtcagaaggacccaagtcgggaatgggatgggccagtctccataaagctgaataaggagctaaaaagtcttattctgaggggtaaaggggtaaagggttcctcggagaggtacctccgaggggtaaacagttgggtaaacagtctctgaagtcagctctgccattttctagctgtatggccctgggcaagtcaatttccttctctgtgctttggtttcctcatccatagaaaggtagaaagggcaaaacaccaaactcttggattacaagagataatttacagaacacccttggcacacagagggcaccatgaaatgtcacgggtgacacagcccccttgtgctcagtccctggcatctctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccggaactgcatgctcaccaccatctgctgcggcaagaacccactgggtgacgatgaggcctctgctaccgtgtccaagacggagacgagccaggtggccccggcctaagacctgcctaggactctgtggccgactataggcgtctcccatcccctacaccttcccccagccacagccatcccaccaggagcagcgcctgtgcagaatgaacgaagtcacataggctccttaattttttttttttttttaagaaataattaatgaggctcctcactcacctgggacagcctgagaagggacatccacca

SEQ ID NO: 2 - protein, 348 aa, Homo sapiens [sequence omitted]

SEQ ID NO: 3 - protein, 13 aa, unidentified
GCCGCCRCCAUGG

SEQ ID NO: 4 - DNA, 25 bp, synthetic construct
  source 1..25: mol_type = other DNA
caccgctgtctacgaagagcccgtg

SEQ ID NO: 5 - DNA, 25 bp, synthetic construct
  source 1..25: mol_type = other DNA
aaaccacgggctcttcgtagacagc

SEQ ID NO: 6 - DNA, 25 bp, synthetic construct
  source 1..25: mol_type = other DNA
cacccgttcatggctgcggctctcg

SEQ ID NO: 7 - DNA, 25 bp, synthetic construct
  source 1..25: mol_type = other DNA
aaaccgagagccgcagccatgaacc

SEQ ID NO: 8 - DNA, 22 bp, synthetic construct
  source 1..22: mol_type = other DNA
tgatatctcgcggatgctgaat

SEQ ID NO: 9 - DNA, 21 bp, synthetic construct
  source 1..21: mol_type = other DNA
tgggcctttagatgagaccaa

SEQ ID NO: 10 - DNA, 23 bp, synthetic construct
  source 1..23: mol_type = other DNA
cgggagcggcgataccgtaaagc

SEQ ID NO: 11 - DNA, 24 bp, synthetic construct
  source 1..24: mol_type = other DNA
gaagcgggaagggactggctgcta

SEQ ID NO: 12 - DNA, 77 bp, synthetic construct
  source 1..77: mol_type = other DNA
aaacgcgacgcgtccccacgggctcttcgtagacagagccgcagccatgaacggcacagagggccccaatttttatg

SEQ ID NO: 13 - DNA, 53 bp, synthetic construct
  source 1..53: mol_type = other DNA
ccaggtggctccagcctaataagtcgacccccacgggctcttcgtagacaggc

SEQ ID NO: 14 - DNA, 47 bp, synthetic construct
  source 1..47: mol_type = other DNA
gtgaacaccggtagacagagccgcagccatggtgagcaagggcgagg

SEQ ID NO: 15 - DNA, 42 bp, synthetic construct
  source 1..42: mol_type = other DNA
ttggcgcgccgtcgacttattacttgtacagctcgtccatgc

SEQ ID NO: 16 - DNA, 59 bp, synthetic construct
  source 1..59: mol_type = other DNA
gaggtctatataagcagagctctctggctaactaagccgcagccatgaacggcacagag

SEQ ID NO: 17 - DNA, 59 bp, synthetic construct
  source 1..59: mol_type = other DNA
gcggctctgtctacgaagagcccgtggggacgcgttaggctggagccacctggctggtc

SEQ ID NO: 18 - DNA, 25 bp, synthetic construct
  source 1..25: mol_type = other DNA
gctgagctcgccaagcagccttggt

SEQ ID NO: 19 - DNA, 25 bp, synthetic construct
  source 1..25: mol_type = other DNA
catgtacgctgccagcatggagaac

SEQ ID NO: 20 - DNA, 53 bp, Homo sapiens
  source 1..53: mol_type = genomic DNA
ctctgtctacgaagagcccgtggggcagcctcgagagccgcagccatgaacgg

SEQ ID NO: 21 - DNA, 53 bp, Homo sapiens
  source 1..53: mol_type = genomic DNA
gagacagatgcttctcgggcaccccgtcggagctctcggcgtcggtacttgcc

SEQ ID NO: 22 - DNA, 60 bp, Mus musculus
  source 1..60: mol_type = genomic DNA
cagccttggtctctgtctacgaagagcccgtggggcagcctcgagagccgcagccatgaa

SEQ ID NO: 23 - DNA, 58 bp, Homo sapiens
  source 1..58: mol_type = genomic DNA
tcgcagcattcttgggtgggagcagccacgggtcagccacaagggccacagccatgaa

SEQ ID NO: 24 - DNA, 58 bp, Macaca fascicularis
  source 1..58: mol_type = genomic DNA
tcgcagcattcttgggtgggagcagacgcggggcagccacaagggccacagccatgaa

SEQ ID NO: 25 - DNA, 56 bp, Homo sapiens
  source 1..56: mol_type = genomic DNA
gagataattttctttcttctctaggtctcagccaaaatgagtgataccccttctac

SEQ ID NO: 26 - DNA, 56 bp, Homo sapiens
  source 1..56: mol_type = genomic DNA
ctctattaaaagaaagaagagatccagagtcggttttactcactatggggaagatg

SEQ ID NO: 27 - DNA, 55 bp, Homo sapiens
  source 1..55: mol_type = genomic DNA
agctgtgctgtgggaagcaacccggactacacttgcaagcatggcgctactgaaa

SEQ ID NO: 28 - DNA, 55 bp, Homo sapiens
  source 1..55: mol_type = genomic DNA
tcgacacgacacccttcgttgggcctgatgtgmccgttcgtaccgcgatgacttt

SEQ ID NO: 29 - DNA, 56 bp, Homo sapiens
  source 1..56: mol_type = genomic DNA
ccaggctcccgcccgccctgagctgcggcctccgcatggaggggccactcactcca

SEQ ID NO: 30 - DNA, 56 bp, Homo sapiens
  source 1..56: mol_type = genomic DNA
ggtccgagggcgggcgggactcgacgccggaggcgtacctccccggtgagtgaggt

SEQ ID NO: 31 - DNA, 7461 bp, synthetic construct
  source 1..7461: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgataccggtgccaccatgtacccatacgatgttccagattacgcttcgccgaagaaaaagcgcaaggtcgaagcgtccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 32 - DNA, 4539 bp, synthetic construct
  source 1..4539: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgataccggtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgtctacgaagagcccgtggttttagagctagaaatagcaagttaaaataaggctagtccgttatcaacttgaaaaagtggcaccgagtcggtgcttttttgacgcgtccgcgtcgacataagaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 33 - DNA, 5634 bp, synthetic construct
  source 1..5634: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccttctagactcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgataccggtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgtctacgaagagcccgtggttttagagctagaaatagcaagttaaaataaggctagtccgttatcaacttgaaaaagtggcaccgagtcggtgcttttttgacgcgtccccacgggctcttcgtagacagagccgcagccatgaacggcacagagggccccaatttttatgtgcccttctccaacgtcacaggcgtggtgcggagccccttcgagcagccgcagtactacctggcggaaccatggcagttctccatgctggcagcgtacatgttcctgctcatcgtgctgggcttccccatcaacttcctcacgctctacgtcaccgtacagcacaagaagctgcgcacacccctcaactacatcctgctcaacttggccgtggctgacctcttcatggtcttcggaggattcaccaccaccctctacacatcactccatggctacttcgtctttgggcccacaggctgtaatctcgagggcttctttgccacacttggaggtgaaatcgccctgtggtccctggtggtcctggccattgagcgctacgtggtggtctgcaagccgatgagcaacttccgcttcggggagaatcacgctatcatgggtgtggtcttcacctggatcatggcgttggcctgtgctgctcccccactcgttggctggtccaggtacatccctgagggcatgcaatgttcatgcgggattgactactacacactcaagcctgaggtcaacaacgaatcctttgtcatctacatgttcgtggtccacttcaccattcctatgatcgtcatcttcttctgctatgggcagctggtcttcacagtcaaggaggcggctgcccagcagcaggagtcagccaccactcagaaggcagagaaggaagtcacccgcatggttatcatcatggtcatcttcttcctgatctgctggcttccctacgccagtgtggccttctacatcttcacccaccagggctccaacttcggccccatcttcatgactctgccagctttctttgctaagagctcttccatctataacccggtcatctacatcatgttgaacaagcagttccggaactgtatgctcaccacgctgtgctgcggcaagaatccactgggagatgacgacgcctctgccaccgcttccaagacggagaccagccaggtggctccagcctaataagtcgacccccacgggctcttcgtagacaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 5306 bp, synthetic construct
  source 1..5306: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgataccggtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgtctacgaagagcccgtggttttagagctagaaatagcaagttaaaataaggctagtccgttatcaacttgaaaaagtggcaccgagtcggtgcttttttgacgcgtccccacgggctcttcgtagacagagccgcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagtcgacccccacgggctcttcgtagacaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 35 - DNA, 5562 bp, synthetic construct
  source 1..5562: mol_type = other DNA
gctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtagacagagccgcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagtcgacccccacgtggggcagcctcgagagccgcagccatgaacggcacagagggccccaatttttatgtgcccttctccaacgtcacaggcgtggtgcggagccccttcgagcagccgcagtactacctggcggaaccatggcagttctccatgctggcagcgtacatgttcctgctcatcgtgctgggcttccccatcaacttcctcacgctctacgtcaccgtacagcacaagaagctgcgcacacccctcaactacatcctgctcaacttggccgtggctgacctcttcatggtcttcggaggattcaccaccaccctctacacatcactccatggctacttcgtctttgggcccacaggctgtaatctcgagggcttctttgccacacttggaggtgaaatcgccctgtggtccctggtggtcctggccattgagcgctacgtggtggtctgcaagccgatgagcaacttccgcttcggggagaatcacgctatcatgggtgtggtcttcacctggatcatggcgttggcctgtgctgctcccccactcgttggctggtccaggtacatccctgagggcatgcaatgttcatgcgggattgactactacacactcaagcctgaggtcaacaacgaatcctttgtcatctacatgttcgtggtccacttcaccattcctatgatcgtcatcttcttctgctatgggcagctggtcttcacagtcaaggaggcggctgcccagcagcaggagtcagccaccactcagaaggcagagaaggaagtcacccgcatggttatcatcatggtcatcttcttcctgatctgctggcttccctacgccagtgtggccttctacatcttcacccaccagggctccaacttcggccccatcttcatgactctgccagctttctttgctaagagctcttccatctataacccggtcatctacatcatgttgaacaagcagttccggaactgtatgctcaccacgctgtgctgcggcaagaatccactgggagatgacgacgcctctgccaccgcttccaagacggagaccagccaggtggctccagcctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcat

SEQ ID NO: 36 - DNA, 5896 bp, synthetic construct
  source 1..5896: mol_type = other DNA
aattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaagccgcagccatgaacggcacagagggccccaatttttatgtgcccttctccaacgtcacaggcgtggtgcggagccccttcgagcagccgcagtactacctggcggaaccatggcagttctccatgctggcagcgtacatgttcctgctcatcgtgctgggcttccccatcaacttcctcacgctctacgtcaccgtacagcacaagaagctgcgcacacccctcaactacatcctgctcaacttggccgtggctgacctcttcatggtcttcggaggattcaccaccaccctctacacatcactccatggctacttcgtctttgggcccacaggctgtaatctcgagggcttctttgccacacttggaggtgaaatcgccctgtggtccctggtggtcctggccattgagcgctacgtggtggtctgcaagccgatgagcaacttccgcttcggggagaatcacgctatcatgggtgtggtcttcacctggatcatggcgttggcctgtgctgctcccccactcgttggctggtccaggtacatccctgagggcatgcaatgttcatgcgggattgactactacacactcaagcctgaggtcaacaacgaatcctttgtcatctacatgttcgtggtccacttcaccattcctatgatcgtcatcttcttctgctatgggcagctggtcttcacagtcaaggaggcggctgcccagcagcaggagtcagccaccactcagaaggcagagaaggaagtcacccgcatggttatcatcatggtcatcttcttcctgatctgctggcttccctacgccagtgtggccttctacatcttcacccaccagggctccaacttcggccccatcttcatgactctgccagctttctttgctaagagctcttccatctataacccggtcatctacatcatgttgaacaagcagttccggaactgtatgctcaccacgctgtgctgcggcaagaatccactgggagatgacgacgcctctgccaccgcttccaagacggagaccagccaggtggctccagcctaacgcgtccccacgggctcttcgtagacagagccgcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctt